(19) United States
(12) Patent Application Publication
(10) Pub. No.: US 2009/0022731 A1
(43) Pub. Date: Jan. 22, 2009

(54) Arthritis-associated B cell gene expression

(75) Inventors: David Johannes von Schack, Arlington, MA (US); Matthew James Whitters, Hudson, MA (US); Kyriaki Dunussi-Johannopoulos, Belmont, MA (US); Mary Collins, Natick, MA (US); Eugene Lee Brown, Newton, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/893,933

(22) Filed: Aug. 16, 2007

Related U.S. Application Data
(60) Provisional application No. 60/840,380, filed on Aug. 25, 2006.

(51) Int. Cl. C07K 16/18; C07K 16/40; A61K 39/395; A61P 19/02

(52) U.S. Cl. 424/145.1; 435/6; 435/455; 435/375; 530/387.1; 530/388.2; 530/388.26; 424/158.1; 424/146.1

(57) ABSTRACT

The invention features methods and compositions benefiting from differential gene expression observed in arthritis-associated B cells.

### CROSS-REFERENCE TO RELATED APPLICATIONS

The present application claims priority to U.S. Provisional Patent Application No. 60/840,380, filed Aug. 25, 2006, which is incorporated herein by reference in its entirety for all purposes.

### TECHNICAL FIELD

This invention relates to arthritis-associated B cell gene expression and methods of using the same for diagnosis and treatment.

### REFERENCE TO SEQUENCE LISTING

This application relates to U.S. Provisional Application No. 60/840,380, filed Aug. 25, 2006, which includes as part of the originally filed subject matter two compact discs, labeled “Copy 1” and “Copy 2,” each disc containing a Sequence Listing. The machine format of each compact disc is IBM-PC and the operating system of each compact disc is MS-Windows. Each of the compact discs includes a single text file, which is named “WYE-068PR.ST25.txt” (583 KB, created Aug. 25, 2006). The contents of the compact discs labeled “Copy 1” and “Copy 2” are hereby incorporated by reference herein in their entireties for all purposes.

### BACKGROUND

There is increasing evidence that B cells play a major role in maintaining autoimmune inflammation by secreting auto-antibodies and cytokines and by presenting antigen to T cells. Recent clinical studies using monoclonal antibodies have shown that B cell depletion is an effective therapeutic approach in patients with rheumatoid arthritis (RA), systemic lupus erythematosus (SLE), and other autoimmune diseases. Diseased joint tissue in rheumatoid arthritis shows infiltration of activated B cells.

### SUMMARY OF THE INVENTION

The present invention features genes whose expression levels in B cells are modulated in autoimmune disease, such as rheumatoid arthritis. Detecting the expression levels of these genes, referred to herein as “B cell activation-regulated genes” or “BCARGs,” can therefore be used to detect or monitor autoimmune disease. Similarly, these genes or gene products can be used as targets for the treatment of autoimmune disease.

BCARGs include each of the genes described herein as differentially expressed in activated B cells in autoimmune disease including, for example, each of the genes listed in Table 1. Some of the genes are more highly expressed (“upregulated”) in these activated B cells: such genes include, for example, PBEF/visfatin, AHCYL1 (S-adenosylhomocysteine hydrolase-like 1), PKC-delta, GNG12, phosphodiesterase 7A, STE-20 like kinase, MAP kinase interacting serine/threonine kinase 2, phosphoinositide-3-kinase regulatory subunit 4 (p150), structural maintenance of chromosomes 5, WD repeat domain 12, exosome component 10, calpain 3, Src-like adaptor protein, CDC-like kinase1, FAM60A, TCTE1L, STUB1/CHIP, MAP4K5, Ro52, and zinc finger protein 106. Others are down-regulated including, for example, copine III, host cell factor regulator 1, Rab3D, biogenesis of lysosome-related organelles complex-1, transmembrane protein 4, acid phosphatase 5, choroideremia, ubiquitin B, and STK10.

Accordingly, in one aspect, the invention provides a method for assessing arthritis-associated B cell activation. The method includes detecting a B cell expression level of one or more genes and comparing the expression level to a reference expression level indicative of the activation state of a B cell. The one or more genes preferably include at least one of the following genes: PBEF/visfatin, AHCYL1 (S-adenoyslhomocysteine hydrolase-like 1), PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, ubiquitin B, or zinc finger protein 106. Because the gene expression detected in the B cell will be that of the endogenous gene, expression of one or more human genes will be detected if the B cell is from a human; expression of one or more mouse genes will be detected if the B cell is from a mouse; etc. In one embodiment, the one or more genes include at least one of the following human genes: AHCYL1 (S-adenoyslhomocysteine hydrolase-like 1), PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, ubiquitin B, or zinc finger protein 106.

Similarly, the invention provides a method of assessing a patient for an indication of an autoimmune response, such as a human. The method includes detecting, in a sample from the patient, a B cell expression level of one or more genes and comparing the expression level to a reference expression level indicative of an immune response in the patient. The immune response can be an autoimmune response such as rheumatoid arthritis. The sample can be a fluid sample such as blood, lymph, or synovium, and B cells can optionally be purified from the sample, such as by fluorescence-activated cell sorting, prior to the detection step. The one or more genes preferably include at least one of the following genes: PBEF/visfatin, AHCYL1 (S-adenoyslhomocysteine hydrolase-like 1), PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, ubiquitin B, or zinc finger protein 106. Because the gene expression detected in the B cell will be that of the endogenous gene, expression of one or more human genes will be detected if the B cell is from a human; expression of one or more mouse genes will be detected if the B cell is from a mouse; etc. In one embodiment, the one or more genes include at least one of the following human genes: AHCYL1 (S-adenoyslhomocysteine hydrolase-like 1), PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, ubiquitin B, or zinc finger protein 106.

The invention also provides methods of treating B cells, e.g., to reduce, prevent, forestall, or counteract B cell activation, by activating a gene or gene product down-regulated in activated B cells, such as copine III, host cell factor regulator 1, Rab3D, biogenesis of lysosome-related organelles complex-1, transmembrane protein 4, acid phosphatase 5, choroideremia, ubiquitin B, or STK10; or by inhibiting a gene or gene product upregulated in activated B cells, such as AHCYL1 (S-adenosylhomocysteine hydrolase-like 1), PKC-delta, GNG12, phosphodiesterase 7A, STE-20 like kinase, MAP kinase interacting serine/threonine kinase 2, phosphoinositide-3-kinase regulatory subunit 4 (p150), structural maintenance of chromosomes 5, WD repeat domain 12, exosome component 10, calpain 3, Src-like adaptor protein, CDC-like kinase1, FAM60A, TCTE1L, STUB1/CHIP, MAP4K5, Ro52, or zinc finger protein 106. The method can optionally incorporate both the activation of one or more downregulated genes or gene products and the inhibition of one or more upregulated genes or gene products. The method can optionally be used to treat rheumatoid arthritis in a patient by treating the patient's B cells.

The invention also provides a method for assessing a treatment for B cells. The method includes detecting, following administration of the treatment, a B cell expression level of one or more genes and comparing the expression level to a reference expression level indicative of B cell activation status, thereby to assess the efficacy of the treatment. For example, the reference expression level can correspond to an expression level prior to administration of the treatment. The one or more genes preferably include at least one of the following genes: AHCYL1, PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, ubiquitin B, and zinc finger protein 106; if the patient is human, the genes are human genes.

The invention also provides antibodies, such as purified antibodies and monoclonal antibodies, that specifically bind gene products overexpressed in activated B cells (e.g. in activated human B cells in autoimmune disease), such as PBEF/visfatin, AHCYL1 (S-adenosylhomocysteine hydrolase-like 1), PKC-delta, GNG12, phosphodiesterase 7A, STE-20 like kinase, MAP kinase interacting serine/threonine kinase 2, phosphoinositide-3-kinase regulatory subunit 4 (p150), structural maintenance of chromosomes 5, WD repeat domain 12, exosome component 10, calpain 3, Src-like adaptor protein, CDC-like kinase1, FAM60A, TCTE1L, STUB1/CHIP, MAP4K5, Ro52, and zinc finger protein 106. The word “antibody,” as used herein, includes full-length antibodies with variable and constant domains, antibody fragments retaining the variable domain or a portion thereof capable of specific binding to antigen, single-chain antibodies, and the like. If the antigen is accessible, administration of the antibody (e.g. to a cell culture or to a human subject) can be used to target an activated B cell (e.g. from a human previously diagnosed with rheumatoid arthritis treatable by targeting an activated B cell with the antibody).

Other features, objects, and advantages of the present invention are apparent in the detailed description that follows. It should be understood, however, that the detailed description, while indicating preferred embodiments of the invention, does so by way of illustration only, not limitation. Various changes and modifications within the scope of the invention will become apparent to those skilled in the art from the detailed description.

### BRIEF DESCRIPTION OF THE DRAWING

FIG. 1 depicts the induction of arthritis symptoms in mice after a collagen immunization on day 0 and a boost on day 21. Clinical scores were assessed on days 28, 35, 42, 49, 56, 63 and 70 and inflammation of the four paws was scored as follows: 0 (no inflammation); 1 (one or two swollen digits); 2 (more than two swollen digits or mild to moderate swelling of paw); 3 (extensive swelling of entire paw); 4 (resolution of swelling, ankylosis of the paw). The scores shown are the individual total scores for each animal. Animals with high clinical scores were sacrificed after day 56. Accordingly, the scores observed at days 63 and 70 represent the continuing progression of the disease in the subset of animals that continued to participate in the experiment on those days, and not a diminution in disease severity following day 56.

### DETAILED DESCRIPTION

Genes that are differentially expressed in disease are useful as markers for the disease and as targets for therapeutic intervention. Using a mouse marker for rheumatoid arthritis, genes that are differentially expressed in B cells were identified. These genes are referred to herein as “B cell activation-regulated genes” or “BCARGs.”

### Identification of BCARGs

To identify novel B cell targets regulated during the course of an autoimmune response resembling human rheumatoid arthritis, temporal changes in the transcriptional profile of B cells in a mouse collagen-induced arthritis (CIA) model were evaluated.

The murine collagen-induced arthritis (CIA) model is a chronic inflammatory disease bearing all the hallmarks of RA, e.g. polyarthritis, synovitis and subsequent cartilage/bone erosions. CIA is induced in susceptible strains of mice, e.g. DBA1/J, by immunization (day 0) with heterologous type II collagen emulsified in complete Freund's adjuvant (CFA), and boost (day 21) with collagen II emulsified in incomplete Freund's adjuvant (IFA). The development of CIA is thought to depend on T cells and disease susceptibility is linked to the MHC region. Following T cell activation an inflammatory cascade involving T cells, B cells, macrophages/monocytes, and activated synoviocytes is triggered.

RNA for gene chip hybridization was extracted from B cells purified from draining lymph nodes at various time points after immunization and boost. Animals injected with only CFA (or IFA for the boost) served as the control group, since they show a similar overall immune response, but will never develop arthritis symptoms in their joints.

BCARGs were identified as described in Example 1. More than 460 genes were identified as having differential expression in B cells of mice developing an anti-collagen immune response. These genes can, of course, be used singly or collectively to evaluate the activation state of mouse B cells and are targets for therapeutic intervention. As CIA is a widely recognized animal model for human rheumatoid arthritis, the corresponding human genes can also be so used. Several of these human genes are summarized in the following table and discussed below.

TABLE 1
B CELL ACTIVATION-REGULATED GENES

| | |
|---|---|
| AHCYL1 | S-adenosylhomocysteine hydrolase-like |
| PKC δ | PKC δ |
| GNG12 | guanine nucleotide binding protein (G Protein), gamma |
| PDE7A | Phosphodiesterase 7A |
| SLK | STE-20 like kinase |
| MAP4K5 | mitogen-activated protein kinase kinase kinase kinase 5 |
| MKNK2 | MAP kinase interacting serine/threonine kinase 2; G protein- |
| | coupled receptor kinase 7 |
| FAM60A | Homo sapiens family with sequence similarity 60, member A |
| TCTE1L | t-complex-associated-testis-expressed 1-like |
| PIK3R4 | phosphoinositide-3-kinase, regulatory subunit 4, p150 |
| STUB1/CHIP | STIP1 homology and U-box containing protein 1 |
| SSA1/TRIM21/Ro52 | tripartite motif-containing 21 |
| SMC5 | SMC5 structural maintenance of chromosomes 5 |
| WDR12 | WD repeat domain 12 |
| EXOSC10 | exosome component 10 |
| CAPN3 | calpain3 |
| SLA, SLAP | Src-like adaptor |
| CLK1 | CDC-like kinase1 |
| HCFC1R1 | host cell factor C1 regulator 1 (XPO1 dependant) |
| RAB3D | RAB3D, member RAS oncogene family |
| BLOC1S1 | biogenesis of lysosome-related organelles complex-1, subunit 1 |
| Copine III, CPNE3 | a calcium-dependent phospholipid-binding protein |
| TMEM4 | transmembrane protein 4 |
| STK10 | serine/threonine kinase 10 |
| ACP5 | acid phosphatase 5 |
| CHM | choroideremia (Rab escort protein 1) |
| PBEF: | Pre-B-cell colony enhancing factor/visfatin |
| UBB | ubiquitin B |
| ZFP106 | zinc finger protein 106 |

### AHCYL1; S-Adenosylhomocysteine Hydrolase-Like 1

The human S-adenosylhomocysteine hydrolase-like 1 (AHCYL1) gene is also known as adenosylhomocysteinase 2 (S-adenosyl-L-homocysteine hydrolase 2) (AdoHcyase 2) and has been mapped to 1p13.2 on human chromosome 1. Its protein and nucleic acid sequences are well known. Representative protein and nucleic acid sequences are shown in the sequence listing as SEQ ID NO:1 and SEQ ID NO:2, respectively. Dekker et al. (2002) Immunogenetics 53(12):993-1001 determined that AHCYL1 mRNA increased markedly during activation of blood and skin dendritic cells (DCs), but was diminished in terminally differentiated tonsil DCs.

### PKC δ

The human nPKC δ gene has been mapped to 3p21.31 on chromosome 3. Protein and nucleic acid sequences corresponding to the human gene are well known; representative nucleic acid and protein sequences are provided as SEQ ID NO:3 and SEQ ID NO:4, respectively.

nPKC δ is involved in B cell signaling and in the regulation of growth, apoptosis, and differentiation of a variety of cell types. nPKC δ is most abundant in B and T lymphocytes of lymphoid organs, cerebrum, and intestine of normal mice. nPKC δ phosphorylates the transcription factor CREB on Ser-133, promoting its activation. By generating mice with a disruption in the Prkcd gene, Miyamoto et al. (2002) Nature 416(6883):865-9 observed that the mice are viable up to 1 year but prone to autoimmune disease, with enlarged lymph nodes and spleens containing numerous germinal centers. Using a mouse model, Mecklenbrauker et al. (2004) Nature 431:456-461 reported a mechanism for the regulation of peripheral B cell survival by serine/threonine protein kinase C-delta: spontaneous death of resting B cells is regulated by nuclear localization of Pkcd that contributes to phosphorylation of histone H2B at serine-14.

### GNG12; Guanine Nucleotide Binding Protein (G Protein), γ12

G protein γ12 has been mapped to 1p31.3 on human chromosome 1. Its protein and nucleic acid sequences are well known; representative nucleic acid and protein sequences are provided as SEQ ID NO: 5 and SEQ ID NO:6. The protein has been reported to be a target of phosphorylation by activated PKC (Morishita et al. (1995) J. Biol. Chem. 270(49):29469-29475).

### PDE7A; Phosphodiesterase 7A

The human PDE7A gene has been mapped to 8q13 on chromosome 8. Protein and nucleic acid sequences for the human gene are known; representative sequences are provided as SEQ ID NO:7 and SEQ ID NO:8. PDE7A is expressed in human proinflammatory and immune cells and has the potential to regulate human T cell function including cytokine production, proliferation and expression of activation markers.

### STE-20 Like Kinase (SLK)

The Step 20 group kinases are proposed to be regulators of MAP kinase cascades. SLK has been mapped to 10q25.1 on human chromosome 10. Nucleic acid and protein sequences of SLK are known; exemplary sequences are provided as SEQ ID NO:9 and SEQ ID NO:10.

### MAP4K5

The human MAP4K5 gene, which has been mapped to 14q11.2-q21 on chromosome 1, is a member of a serine/threonine protein kinase family that is highly similar to yeast SPS1/STE20 kinase. MAP4K5 has been shown to activate Jun kinase in mammalian cells, suggesting a role in the stress response. Two alternatively spliced transcript variants encoding the same protein have been described for this gene. Exemplary nucleic acid sequences are presented as SEQ ID NO:11 and SEQ ID NO:13; the protein translation is presented twice as SEQ ID NO:12 and SEQ ID NO:14. MAP4K5 has kinase activity and activates JNK but not ERK1.

### MKNK2; MAP Kinase Interacting Serine/Threonine Kinase 2; G Protein-Coupled Receptor Kinase 7

The MKNK2 gene has been mapped to 19p13.3 on human chromosome 19. The gene has been reported to have alternatively spliced transcript variants encoding proteins differing at their C-termini; both forms of the protein have been reported to phosphorylate eukaryotic initiation factor eIF4E (Scheper et al. (2003) Mol. Cell. Biol. 23(16):5692-705). Exemplary nucleic acid sequences are presented as SEQ ID NO:15 and SEQ ID NO:17; the corresponding protein sequences are presented as SEQ ID NO:16 and SEQ ID NO:18, respectively.

FAM60A; Homo sapiens Family with Sequence Similarity 60, Member A

FAM60A has been mapped to 12 μl on human chromosome 12. Protein and nucleic acid sequences for human FAM60A are known; representative sequences are provided as SEQ ID NO:19 and SEQ ID NO:20, respectively.

### TCTE1L; DYNLT3; Dynein Light Chain Tctex-Type 3

TCTE1L has been mapped to Xp21 on the human X chromosome. Protein and nucleic acid sequences for human TCTE1L are known; representative sequences are provided as SEQ ID NO:21 and SEQ ID NO:22, respectively.

PIK3R4; Phosphoinositide-3-Kinase, Regulatory Subunit 4, p150

PIK3R4 associates with phosphoinositide-3-kinase in vivo and potentiates its activity in vitro (Panaretou et al. (1997) J. Biol. Chem. 272(4):2477-85). PIK3R4 has been mapped to 3q22.1 on human chromosome 3. Protein and nucleic acid sequences for human PIK3R4 are known; representative sequences are provided as SEQ ID NO:23 and SEQ ID NO:24, respectively.

### STUB1/CHIP; STIP1 Homology and U-Box Containing Protein 1

CHIP has been mapped to 16p33 on human chromosome 16. Protein and nucleic acid sequences for human CHIP are known; representative sequences are provided as SEQ ID NO:25 and SEQ ID NO:26, respectively.

Using an in vitro ubiquitylation assay with recombinant proteins, Jiang et al. (2001) J. Biol. Chem. 276(46):42938-42944 demonstrated that CHIP possesses intrinsic E3 ubiquitin ligase activity and promotes ubiquitylation. This activity was dependent on the C-terminal U box, a domain that shares similarity with yeast UFD2. CHIP interacted functionally and physically with the stress-responsive ubiquitin-conjugating enzyme family UBCH5. A major target of the ubiquitin ligase activity of CHIP was HSC70 itself. CHIP ubiquitylated HSC70, primarily with short, noncanonical multiubiquitin chains, but had no appreciable effect on steady-state levels or half-life of this protein. The authors concluded that CHIP is a bona fide ubiquitin ligase and suggested that U box-containing proteins may constitute a novel family of E3s.

### SSA1/TRIM21/Ro52

Ro/SSA is a ribonucleoprotein that binds to autoantibodies in 35 to 50% of patients with systemic lupus erythematosus (SLE) and in up to 97% of patients with Sjogren syndrome. The protein encoded by this gene is a member of the tripartite motif (TRIM) family. The TRIM motif includes three zinc-binding domains, a RING, a B-box type 1 and a B-box type 2, and a coiled-coil region. This protein is part of the RoSSA ribonucleoprotein which includes a single polypeptide and one of four small RNA molecules. The RoSSA particle localizes to both the cytoplasm and the nucleus. RoSSA interacts with autoantigens in patients with Sjogren syndrome and systemic lupus erythematosus.

The TRIM21 gene has been mapped to 11p15.5 on human chromosome 11. Two alternatively spliced transcript variants for this gene have been described. Representative nucleic acid and protein sequences are provided as SEQ ID NO:27 and SEQ ID NO:28, respectively.

SMC5; Homo sapiens Structural Maintenance of Chromosomes 5

Human SMC5 interacts with human SMC6 (Taylor et al. (2001) Mol. Cell. Biol. 12(6):1583-1594) and with human MMS21 (Potts et al. (2005) Mol. Cell. Biol. 25(16):7021-7032) and likely participates in DNA repair. SMC5 has been mapped to 9q21.11 on human chromosome 9. Protein and nucleic acid sequences for human SMC5 are known; representative sequences are provided as SEQ ID NO:29 and SEQ ID NO:30, respectively.

### WDR12; WD Repeat Domain 12

This gene encodes a member of the WD repeat protein family and has been reported to associate with Pes1 and Bop1 in vivo and to be required for ribosomal RNA processing (Holzel et al. (2005) J. Cell. Biol. 170(3):367-378). The gene has been mapped to 2q33.1 on human chromosome 2. Protein and nucleic acid sequences for human WDR12 are known; representative sequences are provided as SEQ ID NO:31 and SEQ ID NO:32, respectively.

### EXOSC10; Exosome Component 10

The exosome is a complex of 3′-->5′ exoribonucleases that functions in a variety of cellular processes, all concerning the processing or degradation of RNA. The human EXOSC10 gene has been mapped to 1p36.22 on chromosome 1. Protein and nucleic acid sequences for EXOSC10 are known. Representative nucleic acid sequences are provided as SEQ ID NO:33 and SEQ ID NO:35; the corresponding amino acid translations are provided as SEQ ID NO:34 and SEQ ID NO:36, respectively.

### CAPN3; Calpain3

Calpain 3 is an intracellular protease preferentially expressed in B- and T-lymphocytes but poorly expressed in natural killer cells and almost undetectable in polymorphonuclear cells. Mutations in the gene are associated with limb-girdle muscular dystrophies type 2A. The gene is alternatively spliced, with several known transcript variants and associated protein isoforms. Exemplary nucleic acids are presented as SEQ ID NO:37, SEQ ID NO:39, SEQ ID NO:41, SEQ ID NO:43, SEQ ID NO:45, SEQ ID NO:47, SEQ ID NO:49, SEQ ID NO:51, and SEQ ID NO:53; the corresponding polypeptide sequences are presented as SEQ ID NO:38, SEQ ID NO:40, SEQ ID NO:42, SEQ ID NO:44, SEQ ID NO:46, SEQ ID NO:48, SEQ ID NO:50, SEQ ID NO:52, and SEQ ID NO:54, respectively.

### SLA; Src-Like Adaptor; SLAP

Src-like adaptor protein (SLAP) down-regulates expression of the T cell receptor (TCR)-CD3 complex during a specific stage of thymocyte development when the TCR repertoire is selected. Recombinant SLAP has been shown to bind to activated Eck receptor tyrosine kinase. The human gene has been mapped to 8q22.3-qter|8q24 on human chromosome 8. Protein and nucleic acid sequences for human WDR12 are known; representative sequences are provided as SEQ ID NO:55 and SEQ ID NO:56, respectively.

### CDC-Like Kinase1 (CLK1)

CLK1 has been mapped to 2q33 on human chromosome 2. The human CLK1 gene is alternatively spliced, including or omitting exon 4. Protein and nucleic acid sequences for the gene are known. Representative nucleic acid sequences are provided as SEQ ID NO:57 and SEQ ID NO:59; their translation products are provided as SEQ ID NO:58 and SEQ ID NO:60, respectively.

### HCFC1R1; Host Cell Factor C1 Regulator 1 (XPO1 Dependent)

HCFC1R1 has been reported to bind HCF-1, a coactivator for the cellular transcription factors LZIP and GABP, and may regulate HCF-1 by modulating its subcellular localization (Mahajan et al. (2002) J. Biol. Chem. 277(46): 44292-44299). The gene has been mapped to 16p13.3 on human chromosome 16. Protein and nucleic acid sequences for human HCFC1R1 are known. Representative nucleic acid sequences are provided as SEQ ID NO:61, SEQ ID NO:63, and SEQ ID NO:65; their translations are provided as SEQ ID NO:62, SEQ ID NO:64, and SEQ ID NO:66, respectively.

### RAB3D

Rab3D is a known regulator of vesicular trafficking. The gene has been mapped to 19p13.2 of human chromosome 19. Protein and nucleic acid sequences for human RAB3D are known; representative sequences are provided as SEQ ID NO:67 and SEQ ID NO:68, respectively.

### BLOC1S1; Biogenesis of Lysosome-Related Organelles Complex-1, Subunit 1

BLOC1S1 is a subunit of the BLOC-1 (biogenesis of lysosome-related organelles complex-1) complex, “a ubiquitously expressed multisubunit protein complex required for the normal biogenesis of specialized organelles of the endosomal-lysosomal system, such as melanosomes and platelet dense granules” (Starcevic et al. (2004) J. Biol. Chem. 279(27):28393-401). The gene has been mapped to 12q13-q14 of human chromosome 12. Protein and nucleic acid sequences for human BLOC1S1 are known; representative sequences are provided as SEQ ID NO:69 and SEQ ID NO:70, respectively.

### Copine III; CPNE3; a Calcium-Dependent Phospholipid-Binding Protein

CPNE3 appears to possess endogenous kinase activity, although it lacks a classic kinase domain (Caudell et al. (2000) Biochem. 39(42):13034-43). The gene has been mapped to 8q21.3 of human chromosome 8. Protein and nucleic acid sequences for human CPNE3 are known; representative sequences are provided as SEQ ID NO:71 and SEQ ID NO:72, respectively.

### TMEM4; Transmembrane Protein 4

MSAP/TMEM4 is a MIR-interacting protein that enhances neurite outgrowth and increases levels of myosin regulatory light chain (Bornhauser et al. (2003) J. Biol. Chem. 278(37):35412-35420). The TMEM4 gene has been mapped to 12q15 of human chromosome 12. Protein and nucleic acid sequences for human TMEM4 are known; representative sequences are provided as SEQ ID NO:73 and SEQ ID NO:74, respectively.

### STK10

STK10 is a member of the polo-like kinase kinase family and is highly expressed in hematopoietic tissue (Walter et al. (2003) J. Biol. Chem. 278(20): 18221-8). The gene has been mapped to 5q35.1 of human chromosome 5. Protein and nucleic acid sequences for human STK10 are known; representative sequences are provided as SEQ ID NO:75 and SEQ ID NO:76, respectively.

### Acid Phosphatase 5; ACP5; Tartrate-Resistant Acid Phosphatase (TRACP)

ACP5 is an iron-containing glycoprotein that catalyzes the conversion of orthophosphoric monoester to alcohol and orthophosphate. ACP5 is the most basic of the acid phosphatases and is the only form not inhibited by L-tartrate. Serum tartrate-resistant acid phosphatase isoforms have been detected in rheumatoid arthritis, possibly secreted by inflammatory macrophages or dendritic cells (Janckila et al. (2002) Clin. Chem. Acta 320(1-2):49-58). Activated macrophages and osteoclasts express high amounts of tartrate-resistant acid phosphatase. Reactive oxygen species generated by ACP5 may participate in degradation of foreign compounds during antigen presentation in activated macrophages. The gene has been mapped to 19p13.3-p13.2 on human chromosome 19. Protein and nucleic acid sequences for human ACP5 are known; representative sequences are provided as SEQ ID NO:77 and SEQ ID NO:78, respectively.

### CHM; Choroideremia (Rab Escort Protein 1)

The choroideremia gene encodes a protein, the Rab escort protein-1 (REP1), which is involved in membrane trafficking. The gene has been mapped to Xq21.2 on the human X chromosome. Protein and nucleic acid sequences for the human gene are known; representative sequences are provided as SEQ ID NO:79 and SEQ ID NO:80, respectively.

### PBEF

Pre-B-cell colony enhancing factor (PBEF), is a growth factor for early stage B cells and is also known as visfatin and as nicotinamide phosphoribosyltransferase (Nampt). The gene has been mapped to 7q22.2 and its protein and nucleic acid sequences are well known. Representative nucleic acid and protein sequences are shown in the sequence listing as SEQ ID NO:81 and SEQ ID NO:82, respectively. PBEF is upregulated in neutrophils by IL-1β and functions as a novel inhibitor of apoptosis in response to a variety of inflammatory stimuli. PBEF is also an adipocytokine that is highly enriched in the visceral fat of both humans and mice and whose expression level in plasma increases during the development of obesity.

### UBB; Ubiquitin B

The ubiquitin B gene encodes ubiquitin, which is covalently bound to proteins to be degraded. The gene has been mapped to 17p12-p11.2 on human chromosome 17. Protein and nucleic acid sequences for the human gene are known. A representative nucleic acid sequence is provided as SEQ ID NO:83. The translation product is a polyubiquitin precursor with an extra valine as the last amino acid; a representative amino acid sequence of the polyubiquitin precursor is provided as SEQ ID NO:84.

### ZFP106; Zinc Finger Protein 106; SH3BP3; SIRM (Son of Insulin Receptor Mutant)

The ZFP106 gene encodes a zinc finger protein that co-localizes with the nucleolus (Grasberger et al. (2005) Int. J. Biochem. Cell Biol. 37(7):1421-37). The gene has been mapped to 15q15.1 on human chromosome 15. Protein and nucleic acid sequences for the human gene are known; representative sequences are provided as SEQ ID NO:85 and SEQ ID NO:86, respectively.

### Assessment and Treatment

The BCARGs of the present invention can be used to assess arthritis-associated B cell activation and for prediction, diagnosis or prognosis of arthritis or other autoimmune diseases. For example, the genes can be used to identify a patient who is likely to develop rheumatoid arthritis. The genes can also be used to evaluate the progression or effectiveness of a treatment of the autoimmune disease in a patient of interest.

The expression level of the BCARG(s) in a B cell sample of a subject of interest can be compared to a reference expression level of the same gene(s) for predicting, diagnosing or evaluating the progression or treatment of rheumatoid arthritis in the subject of interest. The reference expression level can be prepared using the same type of B cell samples (e.g., from the same source tissue, such as blood, lymph, spleen, or synovium) as the sample of the subject of interest. Both expression levels can be determined using the same preparation procedure or methodology. A reference expression level can be pre-determined or pre-recorded. It can also be prepared concurrently with or after the determination of the expression level of the BCARG of the subject of interest.

A reference expression level employed in the present invention typically includes or consists of a value or range that is suggestive of the expression pattern of the gene in B cell samples of disease-free humans or of patients known to have or to develop rheumatoid arthritis. In one embodiment, a reference expression level comprises the average expression level of the gene in B cell samples of disease-free humans. In another example, a reference expression level comprises the average expression level of the gene in B cell samples of patients known to have or to develop rheumatoid arthritis. Any averaging method can be used, including but not limited to arithmetic means, harmonic means, average of absolute values, average of log-transformed values, and weighted average.

Other types of reference expression levels can also be used in the present invention. For example, a numerical threshold can be used as a reference.

The expression level(s) of the patient of interest and the reference expression level(s) can be constructed in any form. The expression levels can be absolute, normalized, or relative levels. Suitable normalization procedures include, but are not limited to, those used in nucleic acid array gene expression analyses or those described in Hill et al., (2001) Genome Biol., 2:research0055.1-0055.13. In one example, the expression levels are normalized such that the mean is zero and the standard deviation is one. In another example, the expression levels are normalized based on internal or external controls, as appreciated by those skilled in the art. In still another example, the expression levels are normalized against one or more control transcripts with known abundances in B cells.

B cells can be isolated from any suitable source from a subject. The source can be a fluid sample, such as a blood or lymph sample. As one example, blood, such as peripheral blood, can be isolated from a subject; peripheral blood mononuclear cells (PBMCs) can then be isolated using a cell preparation tube (CPT). B cells can be highly enriched by passing the PBMCs over antibody columns that selectively bind non-B cells.

The expression level of the BCARG(s) in a subject of interest can be determined by measuring the RNA transcript level of each of the gene(s) in a B cell sample of the subject. Methods suitable for this purpose include, but are not limited to, quantitative RT-PCR, competitive RT-PCR, real time RT-PCR, differential display RT-PCR, Northern blots, in situ hybridization, slot-blotting, nuclease protection assays, and nucleic acid arrays (including bead arrays).

Detection of the RNA transcript level of a BCARG can incorporate the use of a probe complementary to the RNA or to a corresponding cDNA. A probe capable of hybridizing to a transcript of interest can be labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. In one embodiment, the probes are stably attached to one or more substrate supports. Nucleic acid hybridization or immunoassays can be directly carried out on the substrate support(s). Suitable substrate supports for this purpose include, but are not limited to, glasses, silica, ceramics, nylons, quartz wafers, gels, metals, papers, beads, tubes, fibers, films, membranes, column matrices, or microtiter plate wells.

Hybridization-based methods, such as Northern blots, can include hybridization and washing under stringent or highly stringent conditions. As used herein, “stringent conditions” are at least as stringent as conditions G-L in Table 2. “Highly stringent conditions” are at least as stringent as conditions A-F in Table 2. For each condition, hybridization can be carried out under the corresponding hybridization conditions (“Hybridization Temperature and Buffer”) for about four hours, followed by two 20-minute washes under the corresponding wash conditions (“Wash Temp. and Buffer”).

TABLE 2
STRINGENCY CONDITIONS

| Stringency | Poly-nucleotide | Hybrid Length | Hybridization | Wash Temp. |
|---|---|---|---|---|
| Condition | Hybrid | (bp)¹ | Temperature and Buffer^{H} | nd Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165 M).

The expression profile of the disease gene(s) can also be determined by measuring the protein product level of each of the gene(s) in the B cell sample of the subject of interest. Methods suitable for this purpose include, but are not limited to, immunoassays (e.g., ELISA (enzyme-linked immunosorbent assay), RIA (radioimmunoassay), FACS (fluorescence-activated cell sorter), Western blots, dot blots, immunohistochemistry, antibody-based radioimaging, protein arrays, high-throughput protein sequencing, two-dimensional SDS-polyacrylamide gel electrophoresis, and mass spectrometry. In addition, the biological activity (e.g., enzymatic activity or protein/DNA binding activity) of the protein product encoded by a disease gene can also be used to measure the expression level of the gene in a B cell sample of interest.

The difference or similarity between the expression level of a subject of interest and a reference expression level can be determined by assessing, for example, fold changes, absolute differences, or relative differences after normalization. In one example, the expression level of a BCARG in a subject of interest is considered similar to the corresponding reference expression level if the difference between the two levels is less than 50%, 40%, 30%, 20%, or 10% of the reference expression level. In another example, the expression level of a BCARG in a subject of interest is considered similar to the corresponding reference expression level if the former level falls within the standard deviation (or a multiple or fraction therefore) of the reference expression level.

Where the expression levels of multiple BCARGs of a patient are assessed, an expression profile of the BCARGs in B cells from the patient can be generated and compared to a reference expression profile. The criteria for the overall similarity between the expression profile of a subject of interest and a reference expression profile can be selected such that the accuracy (the ratio of correct calls over the total of correct and incorrect calls) for prediction, diagnosis or assessment is relatively high. For instance, the similarity criteria can be selected such that the accuracy for prediction, diagnosis or assessment is at least 50%, 60%, 70%, 80%, 90%, or more. In one example, an overall similarity call is made if at least 50%, 60%, 70%, 80%, 90%, or more of the components in the expression profile of the subject of interest are considered similar to the corresponding components in the reference expression profile. Different components in the expression profiles may have the same or different weights in comparison. The gene expression-based methods can also be combined with other clinical tests to improve the accuracy of prediction, diagnosis or assessment.

The weighted voting algorithm is capable of assigning a class membership to a subject of interest. See Golub et al., (1999) Science 286:531-537; Slonim et al., (2000) Procs. of the Fourth Annual International Conference on Computational Molecular Biology, Tokyo, Japan, April 8-11, pp. 263-272. Software programs suitable for this purpose include, but are not limited to, the GeneCluster 2 software (Broad Institute, Cambridge, Mass.).

Under one form of the weighted voting analysis, a subject of interest is being assigned to one of two classes (i.e., class 0 and class 1), each class representing a different status (e.g., rheumatoid arthritis or disease-free). For instance, class 0 can include disease-free humans and class 1 includes rheumatoid arthritis patients. A set of BCARGs can be selected from Table 1 to form a classifier (i.e., class predictor). Each gene in the classifier casts a weighted vote for one of the two classes (class 0 or class 1). The vote of gene “g” can be defined as v_{g}=a_{g} (x_{g}−b_{g}), wherein a_{g} equals to P(g,c) and reflects the correlation between the expression level of gene “g” and the class distinction between class 0 and class 1. b_{g} equals to [x0(g)+x1(g)]/2, which is the average of the mean logs of the expression levels of gene “g” in class 0 and class 1. x_{g} represents the normalized log of the expression level of gene “g” in the sample of interest. A positive v_{g} indicates a vote for class 0, and a negative v_{g} indicates a vote for class 1. V0 denotes the sum of all positive votes, and V1 denotes the absolute value of the sum of all negative votes. A prediction strength PS is defined as PS=(V0−V1)/(V0+V1).

Any number of BCARGs can be employed in the present invention. In one embodiment, at least 1, 2, 3, 4, 5, 6, 7, 8, 9, 10, 15, 20, or more genes selected from Table 1 are used for the prediction, diagnosis or evaluation of the effectiveness of a treatment of an immune response in a subject of interest. The disease gene(s) employed in the present invention can be selected to include gene(s) that are upregulated in rheumatoid arthritis patients as compared to disease-free humans, as well as gene(s) that are downregulated in rheumatoid arthritis patients as compared to in disease-free humans.

The BCARGs of the present invention can also be used to identify or test drugs for modulating a B cell-mediated immune response. The ability of a drug candidate to return BCARG expression levels to a state more closely resembling the expression levels in disease-free humans is suggestive of the effectiveness of the drug candidate in autoimmune disease. Methods for screening or evaluating drug candidates are well known in the art. These methods can be carried out either in animal models or during human clinical trials.

The present invention also contemplates expression vectors encoding BCARGs, some of which are under-expressed in B cells of patients with autoimmune disease. By introducing the expression vectors into the patients in need thereof, abnormal expression of these genes can be corrected. Expression vectors and gene delivery techniques suitable for this purpose are well known in the art.

In addition, this invention contemplates sequences that are antisense to BCARGs or expression vectors encoding the same, as some BCARGs are over-expressed in B cells of patients with autoimmune disease. By introducing the antisense sequences or expression vectors encoding the same, abnormal expression of these disease genes can be corrected.

Expression of a BCARG can also be inhibited by RNA interference (“RNAi”). RNAi is a technique used in post transcriptional gene silencing (“PTGS”), in which the targeted gene activity is specifically abolished. In one embodiment, dsRNA of at least about 21 nucleotides is introduced into cells to silence the expression of the target gene.

In addition, the present invention features antibodies that specifically recognize the polypeptides encoded by BCARGs. These antibodies can be administered to patients in need thereof. In one embodiment, an antibody of the present invention can substantially reduce or inhibit the activity of a disease gene. For instance, the antibody can reduce the activity of a BCARG by at least about 20%, 30%, 40%, 50%, 60%, 70%, 80%, 90%, or more. Suitable antibodies for the present invention include, but are not limited to, polyclonal antibodies, monoclonal antibodies, chimeric antibodies, humanized antibodies, single chain antibodies, Fab fragments, or fragments produced by a Fab expression library. In many embodiments, the antibodies of the present invention can bind to the respective BCARG products or other desired antigens with a binding affinity constant Kₐ of at least 10⁶ M⁻¹, 10⁷ M⁻¹, 10⁸ M⁻¹, 10⁹ M⁻¹, or more.

A pharmaceutical composition comprising an antibody or a polynucleotide of the present invention can be prepared. The pharmaceutical composition can be formulated to be compatible with its intended route of administration. Examples of routes of administration include, but are not limited to, parenteral, intravenous, intradermal, subcutaneous, oral, inhalational, transdermal, topical, transmucosal, and rectal administration. Methods for preparing desirable pharmaceutical compositions are well known in the art.

It should be understood that the above-described embodiments and the following examples are given by way of illustration, not limitation. Various changes and modifications within the scope of the present invention will become apparent to those skilled in the art from the present description.

### EXAMPLE 1

RNA for gene chip hybridization was extracted from B cells purified from draining lymph nodes at various time points after immunization and boost. Animals injected with only Complete Freund's Adjuvant (CFA) (and, at the time of boost, Incomplete Freund's Adjuvant (IFA)) served as the control group, since they show a similar overall immune response, but do not develop arthritis symptoms in their joints.

### Arthritis Induction

DBA/1LacJ male mice were immunized intradermally at the base of the tail with CFA alone (control group) or with 100 μg of bovine type II collagen emulsified in CFA (group receiving arthritis induction). Mice were then boosted at day 21 with IFA (control group) or with 100 μg of bovine type II collagen in IFA (group receiving arthritis induction). Clinical scores were assessed on days 28, 35, 42, 49, 56, 63 and 70 post immunization (day 0) and inflammation of the four paws was scored as follows:

0: No inflammation

1: One or two swollen digits

2: More than two swollen digits or mild to moderate swelling

3: Extensive swelling of entire paw

4: Resolution of swelling, ankylosis of the paw

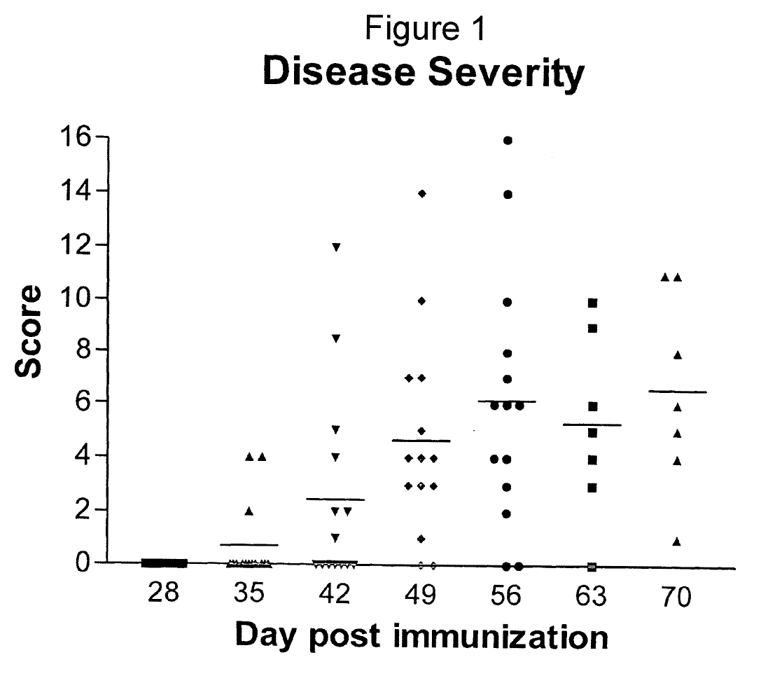

FIG. 1 shows the progression of the disease from a separate cohort of animals run in parallel to the animals used for the transcriptional profiling experiment.

### RNA Isolation, Quantification and Hybridization

B cells were isolated from lymph node samples by FACS sorting using a CD19 antibody. Total RNA was purified from the B cells to very high purity (range 88.5%-99.5%) using a standard Qiagen RNeasy mini-kit procedure. RNA was quantified by UV-Vis absorbance spectra with total RNA amounts mostly around 1 μg (range from 500 ng to 3.5 μg). Because of the low total RNA amounts a two round linear amplification method was employed. Samples were first randomized after the RNA isolation step to avoid the potential of introducing a sample processing bias that would later influence the analysis. The protocol provided with the Affymetrix two-cycle Target Labeling kit was followed for the preparation of biotinylated cRNA.

Standard protocols were used for chip hybridizations to Affymetrix chip MOE430 2.0.

Although target generation for all samples required two rounds of amplification, very few systematic outliers had to be excluded from further analyses. Pearson correlations between replicate samples of a sample group were high (r-Pearson>96% and mostly >98%), indicating robust and reproducible B cell responses at a given time/treatment point.

Unsupervised (two-way) clustering grouped sample replicates primarily next to each other and divided the overall sample set into 3 major branches that could be described as:

naïve (all naïve samples plus samples at from 30 and 35 days post immunization)

pre-response (all 2 days CFA or CFA+CII) and early response (6, 8 and 20 days samples)

post-boost (most samples from after the boost, except very late (30-35 day) samples.

Subsequent transcriptional profiling analysis focused on the timepoints around the boost at day 21, since this treatment provided a robust response in lymph-node derived B cells from CFA+CII treated animals that was stronger than the response from CFA-injected animals alone. A differential gene list was established by looking for differential expression of B cells from CFA+CII treated animals at day 22 (one day after the boost) with the subtraction of genes that were also differential at any of the below conditions:

genes differentially expressed between B cells from CFA treated animals at day 22 vs. day 2 naïve (to focus the differential gene list on collagen-response-specific genes)

genes differentially expressed between B cells from either CFA or CFA+CII treated animals at day 20 vs. day 2 naïve (to avoid genes that are induced in the early response but not necessarily required in B cell activation after the boost, which is essential to create RA-like symptoms in this model)

This analysis revealed 470 genes differentially expressed in B cells following the CFA+CII boost and meeting all other criteria. These genes are presented in Table 3. Table 3 includes, in addition to the gene name: the fold change in gene expression when comparing post-boost B cells to naïve B cells, with positive numbers indicating an increase in expression compared to naïve B cells and negative numbers indicating a decrease in expression; the t-test p-value indicating the significance of the difference in expression levels; the mean expression levels in the naïve samples; and the mean expression levels in the post-boost samples.

TABLE 3
GENES DIFFERENTIALLY EXPRESSED IN B CELLS
AFTER COLLAGEN BOOST

| | Fold | | | |
|---|---|---|---|---|
| | Change | t-Test p- | | |
| | Signed | Value (2 | | |
| | Magnitude | days | | |
| | (2 days | Naïve | | |
| | Naïve vs. | vs. 22 | | |
| | 22 days | days | Mean (2 | Mean (22 |
| | CFA + | CFA + | days | days CFA + |
| Gene Name | collagen) | collagen) | Naïve) | collagen) |
| (ganglioside-induced differentiation-associated- | 1.77 | 4.24E−08 | 170.76 | 302.57 |
| protein 10, pre-B-cell colony-enhancing factor 1) | | | | |
| RIKEN cDNA 2600011C06 gene | 2.76 | 4.66E−08 | 25.06 | 69.11 |
| MAF1 homolog (yeast) | −1.39 | 1.16E−06 | 984.12 | 707.41 |
| per-hexamer repeat gene 4 | 1.8 | 1.18E−06 | 114.73 | 206.59 |
| polymerase (RNA) II (DNA directed) polypeptide L | −1.7 | 1.54E−06 | 283.34 | 166.75 |
| (RIKEN cDNA 2310028O11 gene, similar to | −1.64 | 1.94E−06 | 929.06 | 567.87 |
| protein of fungal metazoan origin like (11.1 kD) | | | | |
| (2C514)) | | | | |
| (eukaryotic translation initiation factor 4A2, | 2.56 | 2.11E−06 | 22.33 | 57.23 |
| expressed sequence AA408556) | | | | |
| (RIKEN cDNA 5330401F18 gene, natural killer | 1.47 | 2.19E−06 | 234.47 | 345.69 |
| tumor recognition sequence) | | | | |
| (ribosomal protein L7, similar to 60S ribosomal | −1.33 | 2.61E−06 | 2699.19 | 2032.57 |
| protein L7) | | | | |
| RIKEN cDNA 2010003O02 gene | −2.19 | 3.82E−06 | 116.62 | 53.16 |
| tubulin, beta 5 | −1.41 | 4.66E−06 | 762.95 | 539.88 |
| (EST AI225873, transportin 3) | 2.11 | 5.05E−06 | 33.71 | 71.21 |
| (RIKEN cDNA 4930511P09 gene, RIKEN cDNA | 1.37 | 5.74E−06 | 282.85 | 387.06 |
| 9130427A09 gene) | | | | |
| deoxynucleotidyltransferase, terminal, interacting | −1.35 | 5.82E−06 | 344.84 | 255.27 |
| protein 1 | | | | |
| zinc finger protein 110 | −1.33 | 6.00E−06 | 344.74 | 258.94 |
| tripartite motif protein 21 | 1.74 | 6.20E−06 | 140.33 | 244.33 |
| RIKEN cDNA 1110007A06 gene | 1.43 | 7.09E−06 | 214.44 | 307.51 |
| Pinin | 1.68 | 7.21E−06 | 225.99 | 380.32 |
| solute carrier family 39 (zinc transporter), | 1.58 | 8.45E−06 | 329.74 | 519.44 |
| member 7 | | | | |
| (DNA segment, Chr 6, ERATO Doi 87, | 1.37 | 8.82E−06 | 157.25 | 215.33 |
| expressed, similar to Tera, teratocarcinoma | | | | |
| expressed, serine rich) | | | | |
| DNA segment, Chr 8, ERATO Doi 325, | 1.67 | 1.17E−05 | 93.25 | 155.37 |
| expressed | | | | |
| RIKEN cDNA 6530403A03 gene | 1.39 | 1.26E−05 | 354.97 | 493.09 |
| mitochondrial ribosomal protein S26 | −1.35 | 1.30E−05 | 430.85 | 318.15 |
| mitochondrial ribosomal protein L15 | −1.35 | 1.60E−05 | 165.72 | 122.48 |
| ORM1-like 2 (S. cerevisiae) | −1.41 | 1.64E−05 | 260.34 | 185.04 |
| thioredoxin 1 | −1.46 | 1.93E−05 | 802.02 | 548.99 |
| host cell factor C1 regulator 1 (XPO1-dependent) | −1.62 | 1.95E−05 | 205.66 | 126.91 |
| (RIKEN cDNA 6030446M11 gene, WAS protein | 1.51 | 2.13E−05 | 134.81 | 203.05 |
| family, member 3, cyclin-dependent kinase 8, | | | | |
| cytochrome P450, family 3, subfamily a, | | | | |
| polypeptide 11, cytochrome P450, family 3, | | | | |
| subfamily a, polypeptide 16, cytochrome P450, | | | | |
| family 3, subfamily a, polypeptide 41, expressed | | | | |
| sequence AL024446) | | | | |
| phosphofructokinase, liver, B-type | 1.4 | 2.22E−05 | 302.55 | 423.16 |
| axin 1 | 1.44 | 2.70E−05 | 270.2 | 388.17 |
| cyclin H | −1.35 | 2.70E−05 | 240.39 | 178.03 |
| mitochondrial ribosomal protein L44 | −1.31 | 2.71E−05 | 404.88 | 308.23 |
| (RNA, U65 small nucleolar, ribosomal protein | 1.32 | 2.91E−05 | 285.46 | 377.77 |
| L12) | | | | |
| dolichol-phosphate (beta-D) mannosyltransferase 2 | −1.43 | 3.17E−05 | 265.62 | 185.25 |
| proteasome (prosome, macropain) subunit, alpha | −1.52 | 3.34E−05 | 1056.23 | 694.12 |
| type 2 | | | | |
| membrane associated DNA binding protein | 1.3 | 3.36E−05 | 107.4 | 139.94 |
| RIKEN cDNA 1500034E06 gene | −1.49 | 3.45E−05 | 172.72 | 115.62 |
| (SEC61, gamma subunit, solute carrier family 37 | −1.63 | 3.45E−05 | 196.36 | 120.72 |
| (glycerol-3-phosphate transporter), member 2) | | | | |
| (RIKEN cDNA A430109H19 gene, ribosomal | −1.59 | 3.67E−05 | 1007.6 | 633.38 |
| protein L36, sulfatase modifying factor 1) | | | | |
| TNFAIP3 interacting protein 2 | 1.51 | 3.75E−05 | 52.86 | 79.89 |
| RIKEN cDNA 2610042O14 gene | −1.31 | 3.87E−05 | 608.55 | 464.83 |
| (CDC23 (cell division cycle 23, yeast, homolog), | 1.48 | 3.94E−05 | 134.48 | 199.7 |
| kinesin family member 20A) | | | | |
| expressed in non-metastatic cells 2, protein | −1.49 | 4.05E−05 | 2225.85 | 1496.79 |
| RIKEN cDNA 2510001I10 gene | 1.43 | 4.13E−05 | 199.76 | 285.91 |
| cDNA sequence BC005662 | −1.37 | 4.27E−05 | 956.58 | 699.49 |
| Ligatin | −1.53 | 4.31E−05 | 182.35 | 119.55 |
| ribosomal protein S28 | −1.39 | 4.34E−05 | 3934.9 | 2837.58 |
| N-ethylmaleimide sensitive fusion protein | 1.33 | 4.37E−05 | 303.78 | 405.37 |
| attachment protein gamma | | | | |
| RIKEN cDNA 2310001H13 gene | 1.43 | 4.41E−05 | 104.46 | 149.66 |
| zinc finger protein 36 | 1.31 | 4.53E−05 | 387.61 | 507.51 |
| heterogeneous nuclear ribonucleoprotein D-like | 1.36 | 4.93E−05 | 711.51 | 964.59 |
| NADH dehydrogenase (ubiquinone) flavoprotein 2 | −1.51 | 5.29E−05 | 436.12 | 289.77 |
| interleukin 7 receptor | 1.89 | 5.30E−05 | 28.46 | 53.84 |
| ribosomal protein S15 | −1.41 | 5.31E−05 | 1547.71 | 1098.44 |
| (histone 3, H2a, histone 3, H2bb) | −1.43 | 5.37E−05 | 224.71 | 157.18 |
| (DNA segment, Chr 10, Brigham & Women's | 1.53 | 5.43E−05 | 258.1 | 395.49 |
| Genetics 1070 expressed, RIKEN cDNA | | | | |
| 5730421K10 gene, similar to heterogeneous | | | | |
| nuclear ribonucleoprotein H3 isoform a, similar to | | | | |
| heterogeneous nuclear ribonucleoprotein H3, | | | | |
| isoform a) | | | | |
| (ribosomal protein S11, similar to 40S ribosomal | −1.38 | 6.29E−05 | 3091.63 | 2233.2 |
| protein S11) | | | | |
| Transcriptional regulator, SIN3B (yeast) | −1.4 | 6.37E−05 | 905.22 | 644.55 |
| (RIKEN cDNA 2310033F14 gene, pre-B-cell | 1.42 | 6.44E−05 | 453.38 | 643.63 |
| leukemia transcription factor interacting protein 1) | | | | |
| RIKEN cDNA 5430405G24 gene | −1.39 | 6.48E−05 | 84.3 | 60.78 |
| mitochondrial ribosomal protein S11 | 1.49 | 6.50E−05 | 34.31 | 51.15 |
| NADH dehydrogenase (ubiquinone) Fe—S protein 8 | −1.51 | 6.73E−05 | 545.4 | 360.76 |
| Myotrophin | −1.47 | 7.52E−05 | 624.31 | 423.48 |
| (similar to Spectrin alpha chain, brain (Spectrin, | −1.33 | 7.84E−05 | 749.48 | 562.39 |
| non-erythroid alpha chain) (Alpha-II spectrin) | | | | |
| (Fodrin alpha chain), spectrin alpha 2) | | | | |
| PRP4 pre-mRNA processing factor 4 homolog B | 1.55 | 8.44E−05 | 159.16 | 246.28 |
| (yeast) | | | | |
| STIP1 homology and U-Box containing protein 1 | 1.32 | 8.74E−05 | 411.61 | 542.5 |
| H2A histone family, member Z | −1.41 | 8.78E−05 | 838.92 | 593.14 |
| (histocompatibility 2, class II, locus DMa, | −1.32 | 8.85E−05 | 1154.56 | 873.62 |
| histocompatibility 2, class II, locus Mb1) | | | | |
| vacuolar protein sorting 54 (yeast) | 1.31 | 8.87E−05 | 304.91 | 400.69 |
| small nuclear ribonucleoprotein D1 | −1.39 | 9.08E−05 | 105.17 | 75.52 |
| (SET translocation, similar to protein | −1.53 | 9.27E−05 | 441.19 | 288.05 |
| phosphatase 2A inhibitor-2 I-2PP2A) | | | | |
| CDK2 (cyclin-dependent kinase 2)-associated | −1.6 | 9.72E−05 | 951.61 | 596.38 |
| protein 1 | | | | |
| (RIKEN cDNA 5830412H02 gene, SWI/SNF | −1.31 | 9.95E−05 | 108.7 | 82.86 |
| related, matrix associated, actin dependent | | | | |
| regulator of chromatin, subfamily e, member 1) | | | | |
| (chromobox homolog 3 (Drosophila HP1 | −1.32 | 1.02E−04 | 248.32 | 187.94 |
| gamma), muted) | | | | |
| adenine phosphoribosyl transferase | −1.45 | 1.03E−04 | 459.58 | 316.52 |
| neutrophil cytosolic factor 4 | −1.43 | 1.06E−04 | 617.18 | 430.35 |
| (RIKEN cDNA 1700001E16 gene, expressed | −1.35 | 1.06E−04 | 144.92 | 107.7 |
| sequence AW986112) | | | | |
| farnesyl diphosphate farnesyl transferase 1 | −1.36 | 1.06E−04 | 92.4 | 68.15 |
| (cDNA sequence BC016198, intercellular | 1.62 | 1.07E−04 | 63.91 | 103.41 |
| adhesion molecule) | | | | |
| CDC-like kinase 1 | 1.34 | 1.07E−04 | 1004 | 1342.3 |
| (RIKEN cDNA A930031G03 gene, expressed | −1.39 | 1.09E−04 | 748.99 | 539.58 |
| sequence C77170, spinocerebellar ataxia 10 | | | | |
| homolog (human)) | | | | |
| p53 and DNA damage regulated 1 | −1.42 | 1.10E−04 | 228.77 | 161.26 |
| (hypothetical protein A130072J07, interferon | 1.34 | 1.11E−04 | 223.65 | 299.95 |
| alpha responsive gene) | | | | |
| (Luc7 homolog (S. cerevisiae)-like, poly A binding | 1.78 | 1.16E−04 | 29.23 | 51.91 |
| protein, cytoplasmic 1) | | | | |
| NADH dehydrogenase (ubiquinone) 1 beta | −1.41 | 1.17E−04 | 349.34 | 247.47 |
| subcomplex 3 | | | | |
| stimulated by retinoic acid 13 | −1.92 | 1.19E−04 | 53.92 | 28.07 |
| glutamyl-prolyl-tRNA synthetase | 1.43 | 1.22E−04 | 152.34 | 218.58 |
| Mitochondrial ribosomal protein L34 | −1.45 | 1.25E−04 | 100.2 | 68.96 |
| LSM4 homolog, U6 small nuclear RNA | −1.41 | 1.25E−04 | 811.26 | 575.47 |
| associated (S. cerevisiae) | | | | |
| zinc finger, FYVE domain containing 27 | 1.39 | 1.25E−04 | 55.61 | 77.54 |
| (RIKEN cDNA 2510019K15 gene, peroxisomal | −1.41 | 1.29E−04 | 111.67 | 78.93 |
| membrane protein 4) | | | | |
| MYST histone acetyltransferase (monocytic | 1.31 | 1.32E−04 | 314.96 | 413.62 |
| leukemia) 3 | | | | |
| acid phosphatase 5, tartrate resistant | −1.45 | 1.34E−04 | 258.81 | 178.69 |
| ribosomal protein S21 | −1.72 | 1.44E−04 | 382.83 | 222.34 |
| RIKEN cDNA 2310020H20 gene | −1.42 | 1.45E−04 | 339.38 | 238.51 |
| (RIKEN cDNA 6030458A19 gene, mitogen | 1.51 | 1.56E−04 | 42.24 | 63.87 |
| activated protein kinase 8) | | | | |
| RIKEN cDNA 2410015N17 gene | −1.43 | 1.61E−04 | 125.57 | 87.56 |
| chloride intracellular channel 4 (mitochondrial) | 1.39 | 1.62E−04 | 100.11 | 139.12 |
| suppressor of variegation 4-20 homolog 1 | 1.31 | 1.67E−04 | 162.48 | 212.9 |
| (Drosophila) | | | | |
| Sin3-associated polypeptide 18 | −1.61 | 1.68E−04 | 362.3 | 225.66 |
| ribosomal protein L19 | −1.45 | 1.74E−04 | 2949.62 | 2028.49 |
| RIKEN cDNA 0610041E09 gene | −1.3 | 1.75E−04 | 300.85 | 230.62 |
| (RIKEN cDNA 1700021K14 gene, RIKEN cDNA | 1.31 | 1.77E−04 | 385.45 | 504.26 |
| 4921523L03 gene, expressed sequence | | | | |
| AU017982, inositol hexaphosphate kinase 1) | | | | |
| RIKEN cDNA 0610009H04 gene | −1.32 | 1.78E−04 | 94.49 | 71.36 |
| RIKEN cDNA 1500001L15 gene | −1.3 | 1.87E−04 | 287.07 | 220.69 |
| protein phosphatase 1G (formerly 2C), | 1.41 | 1.95E−04 | 614.03 | 866.66 |
| magnesium-dependent, gamma isoform | | | | |
| (RIKEN cDNA 1500002C15 gene, RIKEN cDNA | −1.42 | 1.96E−04 | 191.17 | 134.52 |
| 1500011L16 gene) | | | | |
| anaphase-promoting complex subunit 5 | −1.33 | 1.98E−04 | 2026.04 | 1523.79 |
| (RIKEN cDNA D930010H05 gene, casein kinase | 1.34 | 1.98E−04 | 99.87 | 133.37 |
| 1, delta) | | | | |
| myosin, light polypeptide 6, alkali, smooth muscle | −1.45 | 2.05E−04 | 3011.9 | 2080.09 |
| and non-muscle | | | | |
| NADH dehydrogenase (ubiquinone) 1 beta | −1.34 | 2.12E−04 | 233.93 | 174.12 |
| subcomplex, 9 | | | | |
| serine/threonine kinase 10 | −1.49 | 2.15E−04 | 568.54 | 380.97 |
| (active BCR-related gene, expressed sequence | −1.37 | 2.20E−04 | 2055.12 | 1502.36 |
| AI853502, ribosomal protein L18) | | | | |
| RIKEN cDNA 1110039B18 gene | 1.47 | 2.33E−04 | 54.84 | 80.52 |
| solute carrier family 37 (glycerol-3-phosphate | 1.62 | 2.33E−04 | 45.38 | 73.47 |
| transporter), member 1 | | | | |
| COMM domain containing 4 | −1.44 | 2.39E−04 | 529.79 | 367.14 |
| Threonyl-tRNA synthetase-like 1 | 1.56 | 2.42E−04 | 183.49 | 285.72 |
| (RIKEN cDNA 2310040A13 gene, RIKEN cDNA | 1.7 | 2.46E−04 | 30.5 | 51.82 |
| 4930597O21 gene, guanine nucleotide binding | | | | |
| protein (G protein), gamma 12) | | | | |
| transmembrane protein 4 | −1.49 | 2.64E−04 | 148.99 | 99.76 |
| protein kinase inhibitor beta, cAMP dependent, | 1.59 | 2.66E−04 | 153.75 | 244.64 |
| testis specific | | | | |
| RIKEN cDNA C730025P13 gene | −1.44 | 2.82E−04 | 145.94 | 101.55 |
| Parkinson disease (autosomal recessive, early | −1.4 | 2.85E−04 | 748.64 | 533.15 |
| onset) 7 | | | | |
| (RIKEN cDNA 1110059E24 gene, RIKEN cDNA | −1.33 | 2.91E−04 | 327.73 | 246.88 |
| 5730446C15 gene, RIKEN cDNA 9030607L02 | | | | |
| gene) | | | | |
| phosphatidylinositol glycan, class O | 1.43 | 2.95E−04 | 96.22 | 138.03 |
| ribosomal protein L22 | 1.39 | 2.95E−04 | 296.4 | 411.22 |
| NADH dehydrogenase (ubiquinone) 1 beta | −2.15 | 2.96E−04 | 68.55 | 31.93 |
| subcomplex, 2 | | | | |
| (EST X83328, expressed sequence AA408420) | −1.35 | 2.98E−04 | 115.01 | 85.27 |
| (ADP-ribosylation factor-like 6, myc induced | −1.36 | 3.02E−04 | 96.18 | 70.7 |
| nuclear antigen, olfactory receptor 203) | | | | |
| superkiller viralicidic activity 2-like (S. cerevisiae) | 1.89 | 3.07E−04 | 45.83 | 86.49 |
| (peptidylprolyl isomerase A, similar to Peptidyl- | −1.42 | 3.08E−04 | 3202.67 | 2250.98 |
| prolyl cis-trans isomerase A (PPIase) (Rotamase) | | | | |
| (Cyclophilin A) (Cyclosporin A-binding protein) | | | | |
| (SP18)) | | | | |
| (RIKEN cDNA A230103N10 gene, ribosomal | 1.57 | 3.12E−04 | 97.85 | 154.07 |
| protein L30) | | | | |
| surfeit gene 1 | −1.32 | 3.20E−04 | 195.45 | 147.64 |
| exosome component 10 | 1.36 | 3.23E−04 | 50.78 | 69.02 |
| Threonyl-tRNA synthetase | 1.37 | 3.34E−04 | 200.54 | 275.34 |
| keratinocyte associated protein 2 | −1.43 | 3.37E−04 | 593.9 | 414.01 |
| histocompatibility 2, O region alpha locus | −1.41 | 3.38E−04 | 1270.84 | 903.64 |
| (RIKEN cDNA 1700001O11 gene, RIKEN cDNA | −1.31 | 3.39E−04 | 267.89 | 204.12 |
| 2310065K24 gene) | | | | |
| t-complex-associated-testis-expressed 1-like | 1.62 | 3.69E−04 | 30.99 | 50.32 |
| autophagy 5-like (S. cerevisiae) | −1.56 | 3.72E−04 | 89.32 | 57.35 |
| cathepsin H | −1.32 | 3.78E−04 | 1513.12 | 1150 |
| ribosomal protein S28 | −1.39 | 4.10E−04 | 4105.95 | 2959.82 |
| heterogeneous nuclear ribonucleoprotein A2/B1 | 1.77 | 4.12E−04 | 380.19 | 674.03 |
| ribosomal protein S24 | −1.44 | 4.16E−04 | 1709.75 | 1189.21 |
| coiled-coil-helix-coiled-coil-helix domain | −2.32 | 4.41E−04 | 151.11 | 65.15 |
| containing 1 | | | | |
| proteasome (prosome, macropain) subunit, alpha | −1.44 | 4.56E−04 | 1113.36 | 770.58 |
| type 6 | | | | |
| anaphase-promoting complex subunit 5 | −1.36 | 4.57E−04 | 2273.68 | 1666.59 |
| structure specific recognition protein 1 | 1.34 | 4.59E−04 | 983.15 | 1314.59 |
| choroidermia | −1.44 | 4.60E−04 | 112.71 | 78.32 |
| phosphoinositide-3-kinase, catalytic, gamma | −1.71 | 4.61E−04 | 90.57 | 52.95 |
| polypeptide | | | | |
| dynactin 3 | −1.37 | 4.64E−04 | 192.17 | 140.04 |
| similar to phosphatidylserine decarboxylase | 1.31 | 4.72E−04 | 173.82 | 227.44 |
| RIKEN cDNA 2810409H07 gene | 1.68 | 4.76E−04 | 35.57 | 59.74 |
| ornithine decarboxylase antizyme | −1.33 | 4.81E−04 | 3022.85 | 2264.83 |
| (RIKEN cDNA 4930504E06 gene, annexin A9) | −1.41 | 4.99E−04 | 261.57 | 185.69 |
| ubiquitin B | −1.38 | 5.08E−04 | 2894.5 | 2097.09 |
| (ribosomal protein L13, vesicle docking protein) | −1.5 | 5.08E−04 | 2754.65 | 1832.24 |
| diacylglycerol kinase, alpha | 1.35 | 5.10E−04 | 972.26 | 1307.77 |
| (RIKEN cDNA 2400006N03 gene, complement | −1.39 | 5.15E−04 | 1943.41 | 1397.82 |
| component 1, q subcomponent binding protein) | | | | |
| (COX15 homolog, cytochrome c oxidase | 1.9 | 5.24E−04 | 26.41 | 50.1 |
| assembly protein (yeast), ectonucleoside | | | | |
| triphosphate diphosphohydrolase 7) | | | | |
| ubiquitin-conjugating enzyme E2L 3 | −1.32 | 5.27E−04 | 377.9 | 286.2 |
| signal recognition particle receptor (‘docking | 1.3 | 5.72E−04 | 299.58 | 390.24 |
| protein’) | | | | |
| RIKEN cDNA 2610528A15 gene | 1.53 | 5.94E−04 | 179.09 | 274.87 |
| RIKEN cDNA 1110019J04 gene | −1.68 | 5.97E−04 | 334.01 | 198.75 |
| zinc finger protein 330 | 1.45 | 5.97E−04 | 213.79 | 309.52 |
| RIKEN cDNA 5730536A07 gene | −1.8 | 6.00E−04 | 365.81 | 202.98 |
| (RIKEN cDNA A430102J17 gene, WW domain | −1.46 | 6.01E−04 | 285.12 | 195.05 |
| containing adaptor with coiled-coil) | | | | |
| RIKEN cDNA A430005L14 gene | −1.32 | 6.10E−04 | 207.96 | 157.55 |
| Ankyrin repeat and IBR domain containing 1 | 1.35 | 6.10E−04 | 148.88 | 201.19 |
| eukaryotic translation initiation factor 3, subunit 8 | −1.43 | 6.15E−04 | 793.15 | 552.88 |
| (RIKEN cDNA 6030432P03 gene, RNA binding | 1.42 | 6.31E−04 | 254.74 | 360.56 |
| motif, single stranded interacting protein 1, | | | | |
| expressed sequence AI194270, expressed | | | | |
| sequence AW742503) | | | | |
| ubiquitin-like 4 | −1.64 | 6.31E−04 | 216.29 | 132.06 |
| isocitrate dehydrogenase 3 (NAD+), gamma | −1.44 | 6.48E−04 | 804.74 | 559.56 |
| RIKEN cDNA 2310042G06 gene | 1.58 | 6.64E−04 | 108.24 | 171.37 |
| (RIO kinase 3 (yeast), tRNA nucleotidyl | 1.38 | 6.68E−04 | 426.4 | 586.9 |
| transferase, CCA-adding, 1) | | | | |
| biogenesis of lysosome-related organelles | −1.54 | 6.80E−04 | 79.42 | 51.63 |
| complex-1, subunit 1 | | | | |
| (RIKEN cDNA 8030491N06 gene, fractured | −1.46 | 6.84E−04 | 71.1 | 48.57 |
| callus expressed transcript 1) | | | | |
| Guanine nucleotide binding protein, beta 2 | −1.32 | 6.88E−04 | 339.9 | 257.53 |
| RIKEN cDNA 3300001G02 gene | −1.36 | 6.93E−04 | 136.77 | 100.59 |
| (RIKEN cDNA 2210015I05 gene, mucolipin 1) | −1.31 | 6.97E−04 | 87.26 | 66.49 |
| zinc finger protein 161 | 1.35 | 7.08E−04 | 43.43 | 58.6 |
| endothelial differentiation-related factor 1 | −1.42 | 7.14E−04 | 433.54 | 305.9 |
| Tubulin, beta 5 | −1.32 | 7.20E−04 | 1291.6 | 980.41 |
| (ribosomal protein L17, similar to 60S ribosomal | −1.33 | 7.28E−04 | 2231.72 | 1679.18 |
| protein L17 (L23) (Amino acid starvation-induced | | | | |
| protein) (ASI)) | | | | |
| thyrotroph embryonic factor | 1.36 | 7.44E−04 | 145.47 | 198.29 |
| ATP synthase, H+ transporting, mitochondrial F0 | −2.4 | 7.70E−04 | 151.63 | 63.3 |
| complex, subunit c (subunit 9), isoform 1 | | | | |
| Barrier to autointegration factor 1 | −1.31 | 7.74E−04 | 358.01 | 272.82 |
| capping protein (actin filament) muscle Z-line, | −1.31 | 7.74E−04 | 2184.56 | 1670.43 |
| beta | | | | |
| eukaryotic translation initiation factor 2B, subunit | −1.31 | 7.75E−04 | 336.4 | 256.53 |
| 4 delta | | | | |
| phosphodiesterase 7A | 1.37 | 8.47E−04 | 37.88 | 51.84 |
| chondroitin sulfate proteoglycan 6 | 1.51 | 8.63E−04 | 199.32 | 301.38 |
| (ribosomal protein L31, sperm associated antigen | −1.5 | 8.70E−04 | 3163.67 | 2105.08 |
| 6) | | | | |
| (expressed sequence C76686, | 3.89 | 8.79E−04 | 19.16 | 74.56 |
| phosphatidylinositol transfer protein, beta) | | | | |
| (Kruppel-like factor 7 (ubiquitous), RIKEN cDNA | −1.42 | 8.89E−04 | 125.12 | 88.4 |
| D530037H12 gene) | | | | |
| RIKEN cDNA 2900002H16 gene | 6.31 | 9.16E−04 | 8.53 | 53.82 |
| Tubulin, alpha 6 | −1.66 | 9.24E−04 | 397.24 | 239.82 |
| WD repeat domain 9 | 1.34 | 9.26E−04 | 209.11 | 279.9 |
| □sparagines-linked glycosylation 3 homolog | 1.37 | 9.32E−04 | 132.8 | 182.43 |
| (yeast, alpha-1,3-mannosyltransferase) | | | | |
| (RIKEN cDNA A330105O20 gene, golgi | 1.92 | 9.37E−04 | 39.99 | 76.94 |
| autoantigen, golgin subfamily a, 4) | | | | |
| PQ loop repeat containing 1 | −1.36 | 9.56E−04 | 132.77 | 97.57 |
| Dr1 associated protein 1 (negative cofactor 2 | −1.38 | 9.62E−04 | 122.49 | 88.83 |
| alpha) | | | | |
| biliverdin reductase B (flavin reductase (NADPH)) | −1.81 | 9.63E−04 | 179.93 | 99.15 |
| RIKEN cDNA 1600012F09 gene | 1.34 | 9.67E−04 | 51.57 | 69.01 |
| dicarbonyl L-xylulose reductase | −1.33 | 9.75E−04 | 108.8 | 81.81 |
| Copine III | −1.51 | 9.95E−04 | 207.53 | 137.21 |
| NADH dehydrogenase (ubiquinone) Fe—S protein 8 | −1.7 | 1.02E−03 | 222.09 | 130.86 |
| ATP synthase, H+ transporting, mitochondrial F0 | −1.39 | 1.04E−03 | 2256.88 | 1627.14 |
| complex, subunit c (subunit 9), isoform 3 | | | | |
| CDNA sequence BC006933 | 1.45 | 1.04E−03 | 62.39 | 90.49 |
| (RIKEN cDNA 5830445O15 gene, calpain 3) | 1.34 | 1.06E−03 | 274.71 | 369.2 |
| mitochondrial ribosomal protein L12 | −1.49 | 1.08E−03 | 128.88 | 86.76 |
| chromobox homolog 3 (Drosophila HP1 gamma) | −1.31 | 1.10E−03 | 1249.88 | 951.86 |
| (aarF domain containing kinase 2, expressed | −1.31 | 1.13E−03 | 141.21 | 108.03 |
| sequence AI181996, lysocardiolipin | | | | |
| acyltransferase) | | | | |
| splicing factor 3b, subunit 3 | 1.43 | 1.13E−03 | 330.71 | 474.42 |
| RIKEN cDNA 1810073N04 gene | −1.75 | 1.14E−03 | 55.61 | 31.72 |
| sphingomyelin phosphodiesterase, acid-like 3A | −1.42 | 1.15E−03 | 120.38 | 84.78 |
| aminopeptidase puromycin sensitive | 1.34 | 1.15E−03 | 97.7 | 131.29 |
| poly (ADP-ribose) polymerase family, member 6 | 1.59 | 1.20E−03 | 34.01 | 53.93 |
| trinucleotide repeat containing 5 | 1.56 | 1.21E−03 | 83.77 | 130.4 |
| (RIKEN cDNA B230214O09 gene, RIKEN cDNA | 1.45 | 1.22E−03 | 191.16 | 277.78 |
| G430041M01 gene) | | | | |
| (RIKEN cDNA 2010305C02 gene, RIKEN cDNA | −1.36 | 1.23E−03 | 81.83 | 60.11 |
| 2210403K04 gene) | | | | |
| growth arrest specific 5 | 1.42 | 1.24E−03 | 215.38 | 306.62 |
| RIKEN cDNA 2310036D22 gene | −1.32 | 1.24E−03 | 153.88 | 116.24 |
| (WD repeat domain 12, expressed sequence | 1.56 | 1.24E−03 | 63.88 | 99.45 |
| AV258160) | | | | |
| expressed sequence AA960558 | 1.45 | 1.28E−03 | 180.32 | 261.71 |
| pantothenate kinase 1 | −1.42 | 1.28E−03 | 62.23 | 43.83 |
| receptor (TNFRSF)-interacting serine-threonine | 1.33 | 1.29E−03 | 146.54 | 195.39 |
| kinase 1 | | | | |
| annexin A6 | 1.68 | 1.30E−03 | 46.55 | 77.99 |
| KDEL (Lys-Asp-Glu-Leu) endoplasmic reticulum | −1.9 | 1.30E−03 | 205.68 | 108.17 |
| protein retention receptor 2 | | | | |
| ras homolog gene family, member Q | −1.31 | 1.32E−03 | 548.09 | 417.52 |
| (RIKEN cDNA 9230106B05 gene, cyclin D3) | 1.68 | 1.32E−03 | 132.85 | 222.93 |
| translocase of outer mitochondrial membrane 20 | −1.42 | 1.35E−03 | 1062.97 | 749.99 |
| homolog (yeast) | | | | |
| mitochondrial ribosomal protein L13 | −1.52 | 1.35E−03 | 235.39 | 154.83 |
| ribosomal protein, large P2 | −1.53 | 1.35E−03 | 4259.8 | 2790.69 |
| RIKEN cDNA 9430029L20 gene | −1.85 | 1.35E−03 | 392.36 | 211.66 |
| (expressed sequence C76876, ring finger protein | 1.35 | 1.37E−03 | 69.01 | 93.34 |
| 11) | | | | |
| (DNA segment, Chr 6, Massachusetts Institute of | −1.35 | 1.37E−03 | 1989.2 | 1477.84 |
| Technology 97, gene model 1418, (NCBI), gene | | | | |
| model 1502, (NCBI), immunoglobulin kappa | | | | |
| chain variable 21 (V21), immunoglobulin kappa | | | | |
| chain variable 28 (V28), immunoglobulin kappa | | | | |
| chain variable 8 (V8), immunoglobulin kappa | | | | |
| chain, constant region, recombinant | | | | |
| antineuraminidase single chain Ig VH and VL | | | | |
| domains) | | | | |
| protein kinase, AMP-activated, beta 1 non- | −1.41 | 1.39E−03 | 156.04 | 111.03 |
| catalytic subunit | | | | |
| RIKEN cDNA 1810073N04 gene | 1.51 | 1.39E−03 | 180.09 | 271.07 |
| (RIKEN cDNA 1110059H15 gene, similar to | 1.53 | 1.40E−03 | 49.45 | 75.5 |
| CG1530-PA) | | | | |
| COMM domain containing 1 | −1.77 | 1.41E−03 | 110.02 | 62.22 |
| (RIKEN cDNA 2310035P21 gene, programmed | −1.44 | 1.41E−03 | 542.69 | 376.01 |
| cell death 4) | | | | |
| peroxiredoxin 4 | −1.81 | 1.42E−03 | 114.1 | 62.9 |
| RIKEN cDNA 1200002G13 gene | −1.35 | 1.43E−03 | 87.68 | 65.09 |
| solute carrier family 37 (glycerol-3-phosphate | 1.56 | 1.45E−03 | 44.47 | 69.2 |
| transporter), member 3 | | | | |
| ribosomal protein L6 | −1.36 | 1.48E−03 | 3785.23 | 2777.28 |
| protein kinase C, delta | 1.36 | 1.48E−03 | 542.91 | 736.76 |
| small nuclear RNA activating complex, | −1.35 | 1.50E−03 | 209.12 | 154.59 |
| polypeptide 2 | | | | |
| (Metadherin, RIKEN cDNA 9930017N22 gene) | −1.3 | 1.58E−03 | 98.47 | 75.7 |
| cysteinyl-tRNA synthetase | 1.57 | 1.61E−03 | 63.43 | 99.9 |
| RIKEN cDNA 2410018G20 gene | −1.31 | 1.65E−03 | 72.21 | 55.14 |
| Zinc finger, DHHC domain containing 16 | 1.3 | 1.66E−03 | 142.44 | 185.75 |
| ring finger protein 153 | 1.32 | 1.69E−03 | 445.8 | 590.59 |
| (IL2-inducible T-cell kinase, RIKEN cDNA | 1.56 | 1.70E−03 | 37.02 | 57.61 |
| 5830453J16 gene) | | | | |
| WD repeat domain 33 | 3.06 | 1.71E−03 | 17.78 | 54.49 |
| (RIKEN cDNA 5730589L02 gene, TCF3 (E2A) | 1.36 | 1.73E−03 | 68.12 | 92.87 |
| fusion partner, leukocyte receptor cluster (LRC) | | | | |
| member 1, osteoclast associated receptor, | | | | |
| transmembrane channel-like gene family 4) | | | | |
| (expressed sequence T25620, solute carrier | 1.39 | 1.73E−03 | 289.58 | 403.84 |
| family 25, member 28) | | | | |
| Zinc finger RNA binding protein | 1.69 | 1.76E−03 | 31.17 | 52.6 |
| src-like adaptor | 1.45 | 1.76E−03 | 522.58 | 757.15 |
| xeroderma pigmentosum, complementation | 1.38 | 1.76E−03 | 93.67 | 129.46 |
| group C | | | | |
| DnaJ (Hsp40) homolog, subfamily B, member 6 | 1.39 | 1.76E−03 | 159.27 | 221.44 |
| peroxisome biogenesis factor 26 | 1.34 | 1.82E−03 | 90.79 | 121.43 |
| cDNA sequence BC023829 | −1.48 | 1.87E−03 | 167.91 | 113.6 |
| phosphatidylinositol 3 kinase, regulatory subunit, | 1.78 | 1.91E−03 | 121.1 | 216.02 |
| polypeptide 4, p150 | | | | |
| S-adenosylhomocysteine hydrolase-like 1 | 1.77 | 1.91E−03 | 31.05 | 55.11 |
| START domain containing 10 | −1.4 | 1.93E−03 | 152.18 | 108.6 |
| (RIKEN cDNA 5430407F15 gene, heterogeneous | 1.3 | 1.95E−03 | 240.11 | 312.69 |
| nuclear ribonucleoprotein methyltransferase-like | | | | |
| 3 (S. cerevisiae)) | | | | |
| NADH dehydrogenase (ubiquinone) 1, | −1.34 | 2.00E−03 | 632.81 | 473.79 |
| subcomplex unknown, 2 | | | | |
| SEC24 related gene family, member C (S. cerevisiae) | 1.43 | 2.03E−03 | 581.27 | 831.66 |
| tripartite motif protein 34 | 3.11 | 2.03E−03 | 19.65 | 61.11 |
| TAF4A RNA polymerase II, TATA box binding | 1.82 | 2.04E−03 | 44.51 | 81.16 |
| protein (TBP)-associated factor | | | | |
| cDNA sequence BC004728 | −1.47 | 2.04E−03 | 68 | 46.11 |
| (DNA segment, Chr 2, ERATO Doi 554, | −1.42 | 2.05E−03 | 1085.79 | 766.9 |
| expressed, chloride channel, nucleotide- | | | | |
| sensitive, 1A) | | | | |
| DNA segment, Chr 10, ERATO Doi 641, | −1.48 | 2.08E−03 | 51.89 | 34.98 |
| expressed | | | | |
| ATP-binding cassette, sub-family A (ABC1), | 1.5 | 2.15E−03 | 33.9 | 50.87 |
| member 1 | | | | |
| RIKEN cDNA 0910001K20 gene | 1.33 | 2.17E−03 | 123.12 | 164.16 |
| (RIKEN cDNA 2610510B01 gene, similar to | 1.41 | 2.20E−03 | 86.82 | 122.04 |
| Protein C21orf5) | | | | |
| tripeptidyl peptidase II | 1.63 | 2.23E−03 | 30.99 | 50.39 |
| (SH2 domain binding protein 1 (tetratricopeptide | 1.5 | 2.27E−03 | 78.16 | 117.3 |
| repeat containing), expressed sequence | | | | |
| AI785031) | | | | |
| mitogen activated protein kinase kinase kinase 8 | 1.32 | 2.32E−03 | 152.83 | 201.68 |
| (excision repair cross-complementing rodent | −1.51 | 2.32E−03 | 61.31 | 40.48 |
| repair deficiency, complementation group 1, | | | | |
| hypothetical protein 1190028F09) | | | | |
| (RIKEN cDNA 1190002A23 gene, TAR DNA | 1.46 | 2.33E−03 | 259.7 | 379.6 |
| binding protein, mannan-binding lectin serine | | | | |
| protease 2) | | | | |
| (DNA segment, Chr 9, ERATO Doi 338, | 1.8 | 2.44E−03 | 70.31 | 126.29 |
| expressed, caseinolytic protease X (E. coli)) | | | | |
| (DNA segment, Chr 6, ERATO Doi 365, | 1.72 | 2.47E−03 | 42.74 | 73.72 |
| expressed, RIKEN cDNA 2610209C05 gene) | | | | |
| (RNA binding motif protein 12, copine I) | 1.34 | 2.48E−03 | 142.79 | 191.09 |
| (RIKEN cDNA 0610025L06 gene, RIKEN cDNA | −1.53 | 2.51E−03 | 913.2 | 595.47 |
| B930069K15 gene, expressed sequence | | | | |
| AU022928) | | | | |
| RAN binding protein 10 | 1.37 | 2.51E−03 | 116.11 | 159.34 |
| prefoldin 5 | −2.38 | 2.53E−03 | 165.48 | 69.48 |
| (RIKEN cDNA 0610009L18 gene, actin, gamma, | −1.36 | 2.53E−03 | 3558.71 | 2610.61 |
| cytoplasmic 1) | | | | |
| expressed in non-metastatic cells 1, protein | −1.33 | 2.59E−03 | 642.31 | 481.29 |
| (F-box protein 11, mutS homolog 6 (E. coli)) | 1.33 | 2.59E−03 | 152.86 | 203.1 |
| (RIKEN cDNA 2310073E15 gene, regulatory | −1.66 | 2.61E−03 | 71.47 | 43.06 |
| factor X-associated ankyrin-containing protein) | | | | |
| (calcium/calmodulin-dependent protein kinase II, | 2.92 | 2.63E−03 | 18.84 | 54.99 |
| delta, expressed sequence C78441) | | | | |
| transducer of ErbB-2.1 | −1.51 | 2.64E−03 | 59.66 | 39.5 |
| ubiquitin B | −1.36 | 2.64E−03 | 5354.8 | 3930.89 |
| (centaurin, delta 2, gene model 1094, (NCBI)) | 1.61 | 2.68E−03 | 169.29 | 272.87 |
| RIKEN cDNA 1110025L05 gene | −1.31 | 2.68E−03 | 139.6 | 106.31 |
| similar to zinc finger protein 187 | −1.96 | 2.70E−03 | 150.56 | 76.94 |
| expressed sequence AI325464 | 2.33 | 2.71E−03 | 21.46 | 50.05 |
| RIKEN cDNA 2310003L22 gene | −1.4 | 2.74E−03 | 85.03 | 60.76 |
| kelch-like 9 (Drosophila) | −1.42 | 2.77E−03 | 75.78 | 53.33 |
| B lymphoid kinase | 1.32 | 2.80E−03 | 1001.1 | 1323.57 |
| adaptor-related protein complex AP-4, sigma 1 | −1.37 | 2.83E−03 | 82.37 | 60.09 |
| N-methylpurine-DNA glycosylase | −1.34 | 2.88E−03 | 172.32 | 129.06 |
| ribosomal protein S18 | −1.69 | 2.89E−03 | 2822.55 | 1674.5 |
| centrin 3 | 1.3 | 2.91E−03 | 216.77 | 282.13 |
| chemokine (C-C motif) receptor 5 | 1.32 | 2.92E−03 | 86.73 | 114.19 |
| (hypothetical LOC381225, ribosomal protein L15) | −1.41 | 2.94E−03 | 3590.14 | 2545.26 |
| mitochondrial ribosomal protein L47 | −1.38 | 2.95E−03 | 84.33 | 61.23 |
| TAF4A RNA polymerase II, TATA box binding | 1.73 | 2.99E−03 | 30.34 | 52.34 |
| protein (TBP)-associated factor | | | | |
| interferon (alpha and beta) receptor 2 | 1.68 | 3.00E−03 | 53.92 | 90.39 |
| (RIKEN cDNA 2600001M11 gene, RIKEN cDNA | 1.51 | 3.03E−03 | 49.22 | 74.49 |
| B930028L11 gene, Sec61, alpha subunit 2 (S. cerevisiae)) | | | | |
| (RIKEN cDNA 5430410E06 gene, | 1.57 | 3.05E−03 | 1259.43 | 1982.41 |
| histocompatibility 2, D region, histocompatibility | | | | |
| 2, D region locus 1, histocompatibility 2, Q region | | | | |
| locus 2, sperm specific antigen 1) | | | | |
| torsin family 2, member A | −1.31 | 3.07E−03 | 92.82 | 70.85 |
| neuropathy target esterase | 1.5 | 3.08E−03 | 34.52 | 51.92 |
| ubiquitin B | −1.43 | 3.09E−03 | 3296.32 | 2306.23 |
| RIKEN cDNA 1810020E01 gene | −1.69 | 3.15E−03 | 317.96 | 187.9 |
| ATPase family, AAA domain containing 3A | 1.51 | 3.17E−03 | 130.76 | 197.02 |
| methionyl aminopeptidase 1 | 1.61 | 3.17E−03 | 94.53 | 151.77 |
| CD84 antigen | −1.35 | 3.19E−03 | 112.99 | 83.72 |
| (RIKEN cDNA E430007M08 gene, chromobox | −1.68 | 3.21E−03 | 112.23 | 66.88 |
| homolog 1 (Drosophila HP1 beta)) | | | | |
| (RIKEN cDNA 1110054H05 gene, RIKEN cDNA | 1.54 | 3.26E−03 | 57.75 | 89.04 |
| 6230415M23 gene) | | | | |
| 3-monooxgenase/tryptophan 5-monooxgenase | 1.3 | 3.27E−03 | 208.96 | 272.13 |
| activation protein, gamma polypeptide | | | | |
| ELOVL family member 6, elongation of long | −1.44 | 3.31E−03 | 64.1 | 44.37 |
| chain fatty acids (yeast) | | | | |
| (DNA Segment, Chr 15, Mouse Genome | −2.21 | 3.32E−03 | 70.69 | 32.03 |
| Informatics 25, small nuclear ribonucleoprotein | | | | |
| polypeptide G) | | | | |
| nuclear prelamin A recognition factor-like | −1.36 | 3.32E−03 | 217.54 | 160.41 |
| peroxisomal biogenesis factor 12 | 1.34 | 3.33E−03 | 60.03 | 80.22 |
| SH3-domain GRB2-like B1 (endophilin) | −1.42 | 3.33E−03 | 87.63 | 61.59 |
| unc-119 homolog (C. elegans) | −1.38 | 3.38E−03 | 204.95 | 148.52 |
| acidic (leucine-rich) nuclear phosphoprotein 32 | −1.65 | 3.39E−03 | 224.34 | 136.09 |
| family, member A | | | | |
| RIKEN cDNA 4930470D19 gene | 1.33 | 3.41E−03 | 117.64 | 156.75 |
| RIKEN cDNA 2310069I04 gene | 1.43 | 3.42E−03 | 73.65 | 105.66 |
| (DNA segment, Chr 19, ERATO Doi 756, | 1.36 | 3.45E−03 | 51.92 | 70.77 |
| expressed, SMC5 structural maintenance of | | | | |
| chromosomes 5) | | | | |
| amyloid beta (A4) precursor-like protein 2 | 2.22 | 3.46E−03 | 39.94 | 88.78 |
| mago-nashi homolog, proliferation-associated | −1.31 | 3.53E−03 | 327.53 | 250.54 |
| (Drosophila) | | | | |
| (RIKEN cDNA 9430064G09 gene, RIKEN cDNA | 1.76 | 3.55E−03 | 140.78 | 247.29 |
| E230012J19 gene, arginine glutamic acid | | | | |
| dipeptide (RE) repeats) | | | | |
| (RIKEN cDNA 2610039C10 gene, similar to | −1.36 | 3.58E−03 | 224.68 | 165.24 |
| Putative protein C21orf45) | | | | |
| GrpE-like 1, mitochondrial | 1.37 | 3.61E−03 | 105.05 | 144.37 |
| death-associated protein | −1.45 | 3.67E−03 | 131.1 | 90.37 |
| GTPase, IMAP family member 3 | 1.34 | 3.70E−03 | 389.32 | 520.57 |
| RIKEN cDNA 2010011I20 gene | −1.33 | 3.73E−03 | 64.9 | 48.65 |
| mucin 20 | −1.4 | 3.75E−03 | 56.76 | 40.49 |
| hect (homologous to the E6-AP (UBE3A) | 1.57 | 3.76E−03 | 116.9 | 183.9 |
| carboxyl terminus) domain and RCC1 (CHC1)- | | | | |
| like domain (RLD) 2 | | | | |
| lamin B2 | 1.61 | 3.77E−03 | 39.62 | 63.61 |
| cDNA sequence BC004022 | 1.33 | 3.78E−03 | 141.39 | 188.01 |
| vesicle-associated membrane protein 5 | −1.32 | 3.78E−03 | 77.12 | 58.63 |
| MUS81 endonuclease homolog (yeast) | −1.57 | 3.78E−03 | 81.77 | 51.96 |
| RIKEN cDNA 1110028N05 gene | 1.38 | 3.78E−03 | 53.22 | 73.21 |
| (RIKEN cDNA D030074K08 gene, YY1 | −1.38 | 3.83E−03 | 191.3 | 139.13 |
| associated factor 2) | | | | |
| WD repeat domain 8 | −1.42 | 3.86E−03 | 115.8 | 81.49 |
| RAN binding protein 9 | 1.41 | 3.88E−03 | 49.57 | 69.87 |
| Williams Beuren syndrome chromosome region | −1.44 | 3.95E−03 | 231.99 | 161.49 |
| 22 | | | | |
| RIKEN cDNA 6330412F12 gene | 1.38 | 3.99E−03 | 182.58 | 251.08 |
| RIKEN cDNA 4930453N24 gene | −1.31 | 4.07E−03 | 109.07 | 83.26 |
| (ethanol decreased 2, zinc finger, CCHC domain | 1.47 | 4.09E−03 | 70.38 | 103.5 |
| containing 6) | | | | |
| protoporphyrinogen oxidase | 2.25 | 4.19E−03 | 23.6 | 53.05 |
| polymerase (DNA directed), mu | 1.45 | 4.23E−03 | 123.12 | 178.9 |
| phosphatidylinositol-4-phosphate 5-kinase, type 1 | −1.4 | 4.25E−03 | 185.21 | 132.17 |
| beta | | | | |
| Zinc finger, DHHC domain containing 6 | 1.69 | 4.31E−03 | 51.87 | 87.85 |
| (DNA segment, Chr 10, ERATO Doi 214, | −1.32 | 4.31E−03 | 223.17 | 168.93 |
| expressed, RIKEN cDNA 4932439E07 gene) | | | | |
| myotubularin related protein 1 | 1.73 | 4.41E−03 | 57.81 | 100.17 |
| I1-like (S. cerevisiae | 1.31 | 4.46E−03 | 48.44 | 63.35 |
| torsin family 2, member A | −1.57 | 4.47E−03 | 60.18 | 38.34 |
| Sjogren's syndrome nuclear autoantigen 1 | −1.47 | 4.50E−03 | 267.05 | 181.23 |
| WD repeat domain 43 | 1.31 | 4.51E−03 | 311.63 | 407.61 |
| NADH dehydrogenase (ubiquinone) flavoprotein 2 | −1.6 | 4.56E−03 | 288.74 | 181.01 |
| cytochrome c oxidase, subunit VIIIa | −1.41 | 4.63E−03 | 268.28 | 189.66 |
| RIKEN cDNA 0610025L06 gene | −1.34 | 4.69E−03 | 3286.6 | 2457.91 |
| human immunodeficiency virus type I enhancer | 1.39 | 4.70E−03 | 75.62 | 105.3 |
| binding protein 2 | | | | |
| Zinc finger, CW-type with coiled-coil domain 3 | 1.5 | 4.71E−03 | 492.66 | 737.04 |
| (RIKEN cDNA 4432412D15 gene, metal | 1.3 | 4.81E−03 | 64.82 | 84.27 |
| response element binding transcription factor 2) | | | | |
| RIKEN cDNA 3110001A13 gene | −1.79 | 4.86E−03 | 50.56 | 28.28 |
| DNA segment, Chr 3, ERATO Doi 789, | 1.58 | 4.90E−03 | 48.81 | 76.91 |
| expressed | | | | |
| RIKEN cDNA 1110005A23 gene | 1.37 | 4.97E−03 | 44.68 | 61.08 |
| (RIKEN cDNA 1810009N02 gene, RIKEN cDNA | −1.74 | 5.00E−03 | 63.68 | 36.5 |
| 2510022D24 gene) | | | | |
| heterogeneous nuclear ribonucleoprotein M | 1.3 | 5.03E−03 | 681.15 | 886.96 |
| U2 small nuclear RNA auxiliary factor 1-like 4 | −1.46 | 5.10E−03 | 100.26 | 68.59 |
| BcI2-like 2 | 1.53 | 5.11E−03 | 46.89 | 71.8 |
| proteaseome (prosome, macropain) 28 subunit, 3 | 1.59 | 5.15E−03 | 39.31 | 62.36 |
| proteasome (prosome, macropain) 26S subunit, | 1.44 | 5.19E−03 | 224.71 | 323.9 |
| ATPase, 6 | | | | |
| macrophage migration inhibitory factor | −1.32 | 5.25E−03 | 428.09 | 324.64 |
| exosome component 4 | −1.48 | 5.25E−03 | 78.65 | 53.29 |
| (RIKEN cDNA 2810450G17 gene, cDNA | 1.39 | 5.36E−03 | 64.6 | 89.57 |
| sequence BC024479, expressed sequence | | | | |
| AI480624) | | | | |
| DEAD (Asp-Glu-Ala-Asp) box polypeptide 10 | 1.46 | 5.38E−03 | 45.01 | 65.68 |
| sideroflexin 2 | 1.63 | 5.40E−03 | 46.86 | 76.57 |
| deoxyhypusine synthase | −1.42 | 5.40E−03 | 107.25 | 75.4 |
| diazepam binding inhibitor | −1.77 | 5.41E−03 | 52 | 29.39 |
| anaphase promoting complex subunit 13 | −1.47 | 5.46E−03 | 229.3 | 156.27 |
| mitochondrial ribosomal protein L48 | −1.6 | 5.48E−03 | 110.97 | 69.23 |
| RIKEN cDNA 1500035H01 gene | −1.34 | 5.50E−03 | 121.08 | 90.14 |
| RIKEN cDNA 2410018G23 gene | −1.95 | 5.56E−03 | 57.78 | 29.61 |
| RIKEN cDNA 2410026K10 gene | −1.77 | 5.57E−03 | 55.87 | 31.48 |
| golgi autoantigen, golgin subfamily a, 3 | 1.35 | 5.59E−03 | 48.17 | 64.84 |
| (RIKEN cDNA 5730405M06 gene, nuclear | 1.31 | 5.59E−03 | 296.37 | 388.88 |
| receptor co-repressor 1) | | | | |
| GPI-anchored membrane protein 1 | 1.34 | 5.62E−03 | 868.44 | 1162.03 |
| gelsolin | −1.88 | 5.68E−03 | 521.26 | 277.16 |
| zinc finger, DHHC domain containing 3 | 1.45 | 5.84E−03 | 45.99 | 66.72 |
| RIKEN cDNA 2510010F15 gene | −1.5 | 5.84E−03 | 303.51 | 202.04 |
| (ATP/GTP binding protein 1, RIKEN cDNA | 1.3 | 5.86E−03 | 59.48 | 77.42 |
| A230056J06 gene) | | | | |
| RIKEN cDNA 4930588M11 gene | 1.82 | 5.87E−03 | 34.86 | 63.41 |
| 6-phosphogluconolactonase | −1.56 | 5.93E−03 | 249.68 | 159.8 |
| down-regulated by Ctnnb1, a | −1.38 | 6.03E−03 | 207.69 | 150.22 |
| diazepam binding inhibitor | −2.32 | 6.04E−03 | 82.87 | 35.74 |
| cDNA sequence BC019977 | 1.46 | 6.28E−03 | 104.66 | 153.28 |
| ubiquitin-activating enzyme E1-domain | 1.68 | 6.44E−03 | 107.63 | 180.95 |
| containing 1 | | | | |
| WD repeat domain 26 | 1.52 | 6.49E−03 | 34.66 | 52.8 |
| RIKEN cDNA 6330441O12 gene | 1.35 | 6.49E−03 | 103.79 | 140.45 |
| MAP kinase-interacting serine/threonine kinase 2 | 4.51 | 6.50E−03 | 15.05 | 67.89 |
| (DEAH (Asp-Glu-Ala-His) box polypeptide 37, | −1.6 | 6.51E−03 | 62.48 | 39.15 |
| RB-associated KRAB repressor, RIKEN cDNA | | | | |
| 2310010M20 gene, RIKEN cDNA 8030498B09 | | | | |
| gene, cDNA sequence BC019943, interferon | | | | |
| inducible GTPase 2, protein phosphatase 1, | | | | |
| regulatory (inhibitor) subunit 7, survivor of motor | | | | |
| neuron protein interacting protein 1, | | | | |
| synaptotagmin 12) | | | | |
| (RIKEN cDNA 4631427C17 gene, RIKEN cDNA | 1.4 | 6.58E−03 | 60.01 | 84.12 |
| 4930528J11 gene) | | | | |
| (AT rich interactive domain 4B (Rbp1 like), | 1.56 | 6.60E−03 | 184.95 | 288.03 |
| RIKEN cDNA D230040A04 gene) | | | | |
| (expressed sequence AV046995, ubiquitin C) | −1.36 | 6.62E−03 | 1362.89 | 1000.15 |
| PRP39 pre-mRNA processing factor 39 homolog | 1.42 | 6.70E−03 | 88.92 | 126.58 |
| (yeast) | | | | |
| adaptor-related protein complex 3, mu 2 subunit | 1.32 | 6.78E−03 | 47.05 | 61.99 |
| TAF13 RNA polymerase II, TATA box binding | 1.32 | 6.81E−03 | 367.72 | 484.17 |
| protein (TBP)-associated factor | | | | |
| CCR4-NOT transcription complex, subunit 6-like | 1.35 | 6.84E−03 | 131.05 | 176.74 |
| ubiquitin specific protease 25 | 1.4 | 6.87E−03 | 167.89 | 235.5 |
| (DNA Segment, Chr 1, Mouse Genome | −1.42 | 6.90E−03 | 51.66 | 36.28 |
| Informatics 51, SERTA domain containing 3) | | | | |
| gelsolin | −1.44 | 6.93E−03 | 329.02 | 227.96 |
| (DNA segment, Chr 1, ERATO Doi 53, | 1.47 | 6.93E−03 | 49.96 | 73.46 |
| expressed, RIKEN cDNA C130065N10 gene, | | | | |
| cDNA sequence BC049806, hypothetical gene | | | | |
| supported by AK053027) | | | | |
| (gamma-glutamyl carboxylase, methionine | −1.36 | 7.04E−03 | 55.91 | 41.26 |
| adenosyltransferase II, alpha) | | | | |
| RIKEN cDNA 5730511K23 gene | 1.5 | 7.08E−03 | 112.86 | 168.93 |
| RIKEN cDNA 1110058L19 gene | −1.38 | 7.18E−03 | 65.24 | 47.22 |
| glutamyl-prolyl-tRNA synthetase | 1.41 | 7.21E−03 | 64.05 | 90.29 |
| (RIKEN cDNA 0610010D24 gene, expressed | 1.39 | 7.27E−03 | 94.45 | 131.34 |
| sequence AW324073) | | | | |
| transforming growth factor, beta receptor I | 1.33 | 7.30E−03 | 171.28 | 228.46 |
| testis expressed gene 261 | 1.45 | 7.32E−03 | 116.57 | 169.18 |
| DNA segment, Chr 6, Brigham & Women's | −1.39 | 7.33E−03 | 102.23 | 73.59 |
| Genetics 1452 expressed | | | | |
| (RIKEN cDNA 1300007F04 gene, TAO kinase 1) | −1.31 | 7.38E−03 | 87.86 | 66.88 |
| superoxide dismutase 1, soluble | −1.4 | 7.44E−03 | 69.31 | 49.45 |
| (RIKEN cDNA D030074K08 gene, YY1 | −1.32 | 7.47E−03 | 68.32 | 51.95 |
| associated factor 2) | | | | |
| RIKEN cDNA 1110032O16 gene | −1.72 | 7.49E−03 | 161.87 | 94.12 |
| (RIKEN cDNA 1300018I05 gene, mKIAA0646 | 1.36 | 7.54E−03 | 232.43 | 316.63 |
| protein) | | | | |
| (MYB binding protein (P160) 1a, cDNA sequence | 1.42 | 7.55E−03 | 219.07 | 311.9 |
| BC011467) | | | | |
| peroxisomal biogenesis factor 6 | 1.43 | 7.63E−03 | 170.81 | 245.01 |
| hematological and neurological expressed | −1.37 | 7.70E−03 | 166.19 | 120.96 |
| sequence 1 | | | | |
| CD36 antigen | −1.35 | 7.80E−03 | 87.83 | 65.1 |
| RIKEN cDNA 5031436O03 gene | −1.37 | 7.81E−03 | 112.06 | 81.92 |
| (RIKEN cDNA 1110003E01 gene, RIKEN cDNA | −1.3 | 7.88E−03 | 1417.36 | 1088.41 |
| 4930589O11 gene, RIKEN cDNA 5430439C17 | | | | |
| gene) | | | | |
| peptidase (mitochondrial processing) alpha | 1.41 | 8.05E−03 | 98.13 | 138.44 |
| ribosomal protein L31 | −1.42 | 8.14E−03 | 4359.84 | 3076.38 |
| signal sequence receptor, gamma | 1.3 | 8.32E−03 | 436.27 | 568.39 |
| UDP-N-acetylglucosamine pyrophosphorylase 1 | 1.41 | 8.32E−03 | 162.24 | 228.77 |
| (RIKEN cDNA D030040M08 gene, SWI/SNF | 1.4 | 8.33E−03 | 116.32 | 162.44 |
| related, matrix associated, actin dependent | | | | |
| regulator of chromatin, subfamily a, member 5) | | | | |
| tubulin cofactor a | −1.43 | 8.50E−03 | 618.77 | 432.51 |
| (RIKEN cDNA 1810038H16 gene, adaptor- | 1.44 | 8.52E−03 | 144.3 | 207.08 |
| related protein complex AP-4, beta 1) | | | | |
| expressed sequence AW112037 | 1.41 | 8.55E−03 | 274.13 | 385.8 |
| RIKEN cDNA 4833421E05 gene | −1.3 | 8.70E−03 | 61.64 | 47.34 |
| RIKEN cDNA C330021A05 gene | 1.56 | 8.84E−03 | 37.56 | 58.71 |
| COMM domain containing 5 | −1.55 | 8.87E−03 | 110.72 | 71.29 |
| ATPase, H+ transporting, V1 subunit F | −1.41 | 9.02E−03 | 339.83 | 240.6 |
| mitogen-activated protein kinase kinase kinase | 1.35 | 9.12E−03 | 41.33 | 55.88 |
| kinase 5 | | | | |
| zinc finger protein 238 | 1.66 | 9.24E−03 | 486.79 | 809.71 |
| transcription factor B1, mitochondrial | −1.31 | 9.33E−03 | 61.58 | 46.85 |
| B lymphoma Mo-MLV insertion region 1 | 1.4 | 9.35E−03 | 212.85 | 299 |
| Rho GTPase activating protein 6 | −1.58 | 9.47E−03 | 107.9 | 68.08 |
| BCL2/adenovirus E1B 19 kDa-interacting protein | −1.32 | 9.50E−03 | 123 | 93.23 |
| 1, NIP3 | | | | |
| RIKEN cDNA 1700073K01 gene | −1.42 | 9.56E−03 | 58.81 | 41.33 |
| ribosomal protein S14 | −1.44 | 9.56E−03 | 4370.6 | 3042.76 |
| RAB3D, member RAS oncogene family | −1.56 | 9.61E−03 | 51.4 | 33.05 |
| (RIKEN cDNA 4930565N07 gene, RIKEN cDNA | 1.43 | 9.63E−03 | 353.64 | 504.23 |
| 5830484A20 gene) | | | | |
| (RIKEN cDNA C030044O21 gene, elaC | 1.4 | 9.66E−03 | 74.35 | 104.22 |
| homolog 2 (E. coli), expressed sequence | | | | |
| AU040829) | | | | |
| cDNA sequence BC009118 | 1.34 | 9.72E−03 | 110.29 | 147.25 |
| peptidylprolyl isomerase (cyclophilin)-like 4 | 1.3 | 9.74E−03 | 151.79 | 197.58 |
| GTP binding protein 4 | 1.55 | 9.82E−03 | 66.57 | 102.89 |
| (RIKEN cDNA A130026F07 gene, neural | 1.3 | 9.97E−03 | 434.84 | 567.02 |
| precursor cell expressed, developmentally down- | | | | |
| regulated gene 9) | | | | |
| (STE20-like kinase (yeast), expressed sequence | 1.46 | 9.97E−03 | 83.13 | 121.37 |
| C78505) | | | | |

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

We claim:

1. A method of assessing arthritis-associated B cell activation, the method comprising the steps of:
(a) detecting a B cell expression level of one or more genes and
(b) comparing the expression level to a reference expression level indicative of the activation state of a B cell,
wherein the one or more genes are selected from the group consisting of AHCYL1 (S-adenoyslhomocysteine hydrolase-like 1), PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, PBEF/visfatin, ubiquitin B, and zinc finger protein 106.

2. The method of claim 1, wherein the B cell is a human B cell and the one or more genes are human genes.

3. A method of assessing a patient for an indication of an autoimmune response, the method comprising the steps of:
(a) detecting, in a sample from the patient, a B cell expression level of one or more genes and
(b) comparing the expression level to a reference expression level indicative of an immune response in the patient,
wherein the one or more genes are selected from the group consisting of AHCYL1, PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, PBEF/visfatin, ubiquitin B, and zinc finger protein 106.

4. The method of claim 3, wherein the immune response is an autoimmune response.

5. The method of claim 4, wherein the autoimmune response is rheumatoid arthritis.

6. The method of claim 3, wherein the sample is a fluid sample selected from the group consisting of blood, lymph, and synovium.

7. The method of claim 6, further comprising the step of purifying B cells from the sample prior to step (a).

8. The method of claim 4, wherein the patient is a human patient and the one or more genes are human genes.

9. A method of treating B cells, the method comprising the step of activating a gene or gene product selected from the group consisting of copine III, host cell factor regulator 1, Rab3D, biogenesis of lysosome-related organelles complex-1, transmembrane protein 4, acid phosphatase 5, choroideremia, ubiquitin B, and/or STK10 or inhibiting a gene or gene product selected from the group consisting of AHCYL1 (S-adenosylhomocysteine hydrolase-like 1), PKC-delta, GNG12, phosphodiesterase 7A, STE-20 like kinase, MAP kinase interacting serine/threonine kinase 2, phosphoinositide-3-kinase regulatory subunit 4 (p150), structural maintenance of chromosomes 5, WD repeat domain 12, exosome component 10, calpain 3, Src-like adaptor protein, CDC-like kinase1, FAM60A, TCTE1L, STUB1/CHIP, MAP4K5, Ro52, and zinc finger protein 106.

10. The method of claim 9, wherein the method comprises activating a gene or gene product selected from the group consisting of copine III, host cell factor regulator 1, Rab3D, biogenesis of lysosome-related organelles complex-1, transmembrane protein 4, acid phosphatase 5, choroideremia, ubiquitin B, and STK10 by expression of a nucleic acid encoding the gene product.

11. The method of claim 9, wherein the method comprises inhibiting a gene selected from the group consisting of AHCYL1 (S-adenosylhomocysteine hydrolase-like 1), PKC-delta, GNG12, phosphodiesterase 7A, STE-20 like kinase, MAP kinase interacting serine/threonine kinase 2, phosphoinositide-3-kinase regulatory subunit 4 (p150), structural maintenance of chromosomes 5, WD repeat domain 12, exosome component 10, calpain 3, Src-like adaptor protein, CDC-like kinase1, FAM60A, TCTE1L, STUB1/CHIP, MAP4K5, Ro52, and zinc finger protein 106 by expression of a nucleic acid inhibiting expression of the gene.

12. A method of treating rheumatoid arthritis in a patient, the method comprising the step of treating B cells in the patient according to the method of claim 9.

13. A method of assessing a treatment for B cells, the method comprising the step of detecting, following administration of the treatment, a B cell expression level of one or more genes and comparing the expression level to a reference expression level indicative of B cell activation status, thereby to assess the efficacy of the treatment, wherein the one or more genes are selected from the group consisting of AHCYL1, PKC-delta, GNG12, phosphodiesterase 7A, FAM60A, TCTE1L, STUB1/CHIP, copine III, STK10, STE-20 like kinase, MAP4K5, MAP kinase interacting serine/threonine kinase 2, phosphoinositide-3-kinase regulatory subunit 4 (p150), Ro52, structural maintenance of chromosomes 5, WD repeat domain 12, exosome component 10, calpain 3, Src-like adaptor protein, CDC-like kinase1, host cell factor C1 regulator 1, Rab3D, biogenesis of lysosome-related organelles complex-1, transmembrane protein 4, acid phosphatase 5, choroideremia, ubiquitin B, and zinc finger protein 106.

14. The method of claim 13, wherein the reference expression level corresponds to an expression level prior to administration of the treatment.

15. The method of claim 14, wherein the one or more genes are human genes.

16. A purified or monoclonal antibody that specifically binds a gene product selected from the group consisting of PBEF/visfatin, AHCYL1 (S-adenosylhomocysteine hydrolase-like 1), PKC-delta, GNG12, phosphodiesterase 7A, STE-20 like kinase, MAP kinase interacting serine/threonine kinase 2, phosphoinositide-3-kinase regulatory subunit 4 (p150), structural maintenance of chromosomes 5, WD repeat domain 12, exosome component 10, calpain 3, Src-like adaptor protein, CDC-like kinase1, FAM60A, TCTE1L, STUB1/CHIP, MAP4K5, Ro52, and zinc finger protein 106.

17. The antibody of claim 16, wherein the gene product is a human gene product.

18. A method of targeting an activated B cell, the method comprising the step of administering the antibody of claim 17.

19. The method of claim 18, wherein the antibody is administered to a human.

20. The method of claim 19, wherein the human had previously been diagnosed with rheumatoid arthritis treatable by targeting an activated B cell with the antibody.


SEQUENCE LISTING (86 sequences)

SEQ ID NO: 1 - DNA, 4024 bp, Homo sapiens
  CDS (368)..(1960)
cagagtgccctttctccccgcctcttccccctcccgggagctgccagtacttgacgtggcgtcaccgccctctaccctcgctttgcgtgcgtgtttgcgtacagcggaggtggcggcgcgggcaggtcggagctcggagctgctgttctggttctcttgtggccgccgtcgctgtccggctgccttgggctgccgaacagacaaggcgtgggccacagcacctcagaagccgacgcagctcgacgcaggggccggcaggagggtgggcgatcgcgtgtcggagggcgccgcgcgggcaggcgggcgggcgccagagggggaaagaggcgggggcggcgggtcagccgctggccgggccggccggggaatgtcgatgcctgacgcgatgccgctgcccggggtcggggaggagctgaagcaggccaaggagatcgaggacgccgagaagtactccttcatggccaccgtcaccaaggcgcccaagaagcaaatccagtttgctgatgacatgcaggagttcaccaaattccccaccaaaactggccgaagatctttgtctcgctcgatctcacagtcctccactgacagctacagttcagctgcatcctacacagatagctctgatgatgaggtttctccccgagagaagcagcaaaccaactccaagggcagcagcaatttctgtgtgaagaacatcaagcaggcagaatttggacgccgggagattgagattgcagagcaagacatgtctgctctgatttcactcaggaaacgtgctcagggggagaagcccttggctggtgctaaaatagtgggctgtacacacatcacagcccagacagcggtgttgattgagacactctgtgccctgggggctcagtgccgctggtctgcttgtaacatctactcaactcagaatgaagtagctgcagcactggctgaggctggagttgcagtgttcgcttggaagggcgagtcagaagatgacttctggtggtgtattgaccgctgtgtgaacatggatgggtggcaggccaacatgatcctggatgatgggggagacttaacccactgggtttataagaagtatccaaacgtgtttaagaagatccgaggcattgtggaagagagcgtgactggtgttcacaggctgtatcagctctccaaagctgggaagctctgtgttccggccatgaacgtcaatgattctgttaccaaacagaagtttgataacttgtactgctgccgagaatccattttggatggcctgaagaggaccacagatgtgatgtttggtgggaaacaagtggtggtgtgtggctatggtgaggtaggcaagggctgctgtgctgctctcaaagctcttggagcaattgtctacattaccgaaatcgaccccatctgtgctctgcaggcctgcatggatgggttcagggtggtaaagctaaatgaagtcatccggcaagtcgatgtcgtaataacttgcacaggaaataagaatgtagtgacacgggagcacttggatcgcatgaaaaacagttgtatcgtatgcaatatgggccactccaacacagaaatcgatgtgaccagcctccgcactccggagctgacgtgggagcgagtacgttctcaggtggaccatgtcatctggccagatggcaaacgagttgtcctcctggcagagggtcgtctactcaatttgagctgctccacagttcccacctttgttctgtccatcacagccacaacacaggctttggcactgatagaactctataatgcacccgaggggcgatacaagcaggatgtgtacttgcttcctaagaaaatggatgaatacgttgccagcttgcatctgccatcatttgatgcccaccttacagagctgacagatgaccaagcaaaatatctgggactcaacaaaaatgggccattcaaacctaattattacagatactaatggaccatactaccaaggaccagtccacctgaaccacacactctaaagaaatattttttaagataacttttattttcttcttactcctttcctcttgatttttttcctataatttcattcttgttttttcatctcattatccaagttctgcagaccacacaggaacttgcttcatggctctttagatgaaatagaagttcagggttcctcactctagtcactaaagaaggattttactctcccagcccagaaaggtgattctttctttaccatttctggggactttagtcttaattaggtaccttattaacaggaaatgctaaggtaccttctctgtggaacaatctgcaatgtctaaatcgccttaaaagagcccatttcttagctgctgaaatcagtgctctttcacttcttcagagaagcagggatggtacctacccggcaggtaggttagatgtgggtggtgcatgttaatttcccttagaagttccaagccctgtttcctgcgtaaaggtggtatgtccagttcagagatgtgtataatgagcatggcttgttaagatcaggaggcccacttggatttatagtatagcccttcctccactcccaccagacttgctcatttttcgagtttttaactagactacactctattgagtttaattttgtcctctaggatttatttctgttgtccaaaaaaaaaaaaaaagaaaagaaaaattaaggagaatttttggtgttaatgctgaggaattgcttgagtggttagttgttaccaatttctcttttgaacctttggagctaaggatgctgagtctagagaaatgctagtctcaagccctgttaagtccctctgtttctagcccgtagttcatagcatcagtgaactggagccacaacagcaaattctatcagctgtgtaccatacagcttgtgctgaaggcgaatttcttgagccattactcagtataaagcactgagttctatctttaggatttatctttaagagcaaatttctggtcagctgtgcttctgcaacctaaaatatttaaagggaggtaggtgtgggcaggaggaggaatgataaattgggccagggcaagaaaaatctagcttcatataatttgtctgggactatacaccctatataatgttagttttacagaagtaatatgacttttgattgctacataccacaaagagtttatgaactgagatcataaagggcaactgatgtgtgaagaaagtagtcagtacatcctggctcatgctctgaaagaatatccagagaggctctctcaaagatcagggagatgtattcccatgccatgcaccctgcttcccagcatttctgcatggtcaagtgagctttatgctcatgagctttaagtatataattatccaggattttaaatcctcaacttgttctagcttgtgatccctcaaagttgggtcatacgttagtgctagatactagaaattttcacttttccactgatcagagagacagacattaaaaacaaaaatagaagaaaggaaagctttcaccctgcagcttcttagcagggaacaattgtcttgccaaaacttttttcccttttctctcccattttcttttacccaatcccttcttactccttgccagtgtgaccatgctttcttctctgtagatgttaacagttaaggcctattttcctcgggcacttaaccaaccaatcagaacaccacatctgttaggggaggtaacctggccaacagtgtatccatcacgttagccctgctggagggaagggacccacattcacctgccctctgacctgccccttgatcccatatctattaccgtgtccataggaataataggtaagggctctgtctctgtcaagccatgtaacaaaggacactgttaaaaaaaaaaaaaagtctggcatcagagggagcatgtggagagcaacttgggaagaacaagttcattttgtattgaatgatttttaatgaatgcaatattaatccttgcagatgagcaataatcattaaaatcgattaaaatgataagaccttaaa

SEQ ID NO: 2 - protein, 530 aa, Homo sapiens
MSMPDAMPLPGVGEELKQAKEIEDAEKYSFMATVTKAPKKQIQFADDMQEFTKFPTKTGRRSLSRSISQSSTDSYSSAASYTDSSDDEVSPREKQQTNSKGSSNFCVKNIKQAEFGRREIEIAEQDMSALISLRKRAQGEKPLAGAKIVGCTHITAQTAVLIETLCALGAQCRWSACNIYSTQNEVAAALAEAGVAVFAWKGESEDDFWWCIDRCVNMDGWQANMILDDGGDLTHWVYKKYPNVFKKIRGIVEESVTGVHRLYQLSKAGKLCVPAMNVNDSVTKQKFDNLYCCRESILDGLKRTTDVMFGGKQVVVCGYGEVGKGCCAALKALGAIVYITEIDPICALQACMDGFRVVKLNEVIRQVDVVITCTGNKNVVTREHLDRMKNSCIVCNMGHSNTEIDVTSLRTPELTWERVRSQVDHVIWPDGKRVVLLAEGRLLNLSCSTVPTFVLSITATTQALALIELYNAPEGRYKQDVYLLPKKMDEYVASLHLPSFDAHLTELTDDQAKYLGLNKNGPFKPNYYRY

SEQ ID NO: 3 - DNA, 2850 bp, Homo sapiens
  CDS (354)..(2384)
tcgcacttccgtgtgccgcggcgccggagcccgaggcggctgtagcccacatctcccgagcgacccccggcgcccgcccgccgcgcggaggcccgggccacacctcactggccgcttggcccatcccagtcagcgccgcgccgaaccccgtccgcgcgcgccggggagcggcgcccccgccgctgccgccgcgacccttggcgcctgcccctgcaacgggaggtctgcagggaactggccaggcaagggggcaggcccgtttctcctggtggttggtgcgttgtagcagcagcgggagccaggactaaggacaagcaggagctgggagccccagccccactgcaggccccaccatggcgccgttcctgcgcatcgccttcaactcctatgagctgggctccctgcaggccgaggacgaggcgaaccagcccttctgtgccgtgaagatgaaggaggcgctcagcacagagcgtgggaaaacactggtgcagaagaagccgaccatgtatcctgagtggaagtcgacgttcgatgcccacatctatgaggggcgcgtcatccagattgtgctaatgcgggcagcagaggagccagtgtctgaggtgaccgtgggtgtgtcggtgctggccgagcgctgcaagaagaacaatggcaaggctgagttctggctggacctgcagcctcaggccaaggtgttgatgtctgttcagtatttcctggaggacgtggattgcaaacagtctatgcgcagtgaggacgaggccaagttcccaacgatgaaccgccgcggagccatcaaacaggccaaaatccactacatcaagaaccatgagtttatcgccaccttctttgggcaacccaccttctgttctgtgtgcaaagactttgtctggggcctcaacaagcaaggctacaaatgcaggcaatgtaacgctgccatccacaagaaatgcatcgacaagatcatcggcagatgcactggcaccgcggccaacagccgggacactatattccagaaagaacgcttcaacatcgacatgccgcaccgcttcaaggttcacaactacatgagccccaccttctgtgaccactgcggcagcctgctctggggactggtgaagcagggattaaagtgtgaagactgcggcatgaatgtgcaccataaatgccgggagaaggtggccaacctctgcggcatcaaccagaagcttttggctgaggccttgaaccaagtcacccagagagcctcccggagatcagactcagcctcctcagagcctgttgggatatatcagggtttcgagaagaagaccggagttgctggggaggacatgcaagacaacagtgggacctacggcaagatctgggagggcagcagcaagtgcaacatcaacaacttcatcttccacaaggtcctgggcaaaggcagcttcgggaaggtgctgcttggagagctgaagggcagaggagagtactttgccatcaaggccctcaagaaggatgtggtcctgatcgacgacgacgtggagtgcaccatggttgagaagcgggtgctgacacttgccgcagagaatccctttctcacccacctcatctgcaccttccagaccaaggaccacctgttctttgtgatggagttcctcaacgggggggacctgatgtaccacatccaggacaaaggccgctttgaactctaccgtgccacgttttatgccgctgagataatgtgtggactgcagtttctacacagcaagggcatcatttacagggacctcaaactggacaatgtgctgctggaccgggatggccacatcaagattgccgactttgggatgtgcaaagagaacatattcggggagagccgggccagcaccttctgcggcacccctgactatatcgcccctgagatcctacagggcctgaagtacacattctctgtggactggtggtctttcggggtccttctgtacgagatgctcattggccagtcccccttccatggtgatgatgaggatgaactcttcgagtccatccgtgtggacacgccacattatccccgctggatcaccaaggagtccaaggacatcctggagaagctctttgaaagggaaccaaccaagaggctgggagtgaccggaaacatcaaaatccaccccttcttcaagaccataaactggactctgctggaaaagcggaggttggagccacctttcaggcccaaagtgaagtcacccagagactacagtaactttgaccaggagttcctgaacgagaaggcgcgcctctcctacagcgacaagaacctcatcgactccatggaccagtctgcattcgctggcttctcctttgtgaaccccaaattcgagcacctcctggaagattgaggttcctggacagatcaggctagccctgccctccacccacacctgcccgctccccacgataagcaccagtgggactgtggtgacttctgctgctggccccgcccctgcccccagagcgtccttggctgccgtctggccgggctctcatggtacttcctctgtgaactgtgtgtgaatctgcttttcctctgccttcggagggaaattgtaaatcctgtgtttcattacttgaatgtagttatctattgaaaatatatattatatacatagacatatatatatatataataggctgtatatattgctcagtagagaaaaaccatgggggactggtgatatgttgatctttttcaaaaaaatatatatatgacaaaaaaaaaaaaaaaggagcacaagctgtttgaaccaccaggtttatttgtgtgtctaaataaacaccaaatagtaccaaaaaaaaaaaaaaaaa

SEQ ID NO: 4 - protein, 676 aa, Homo sapiens
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 5 - DNA, 4399 bp, Homo sapiens
  CDS (159)..(377)
ggaggagaggtcggagccgtctccaggagcccttagagaccgagtcccggcggcgacggcggggcagcgcaccggcaggcggattcattccacttaaaacctgaaaacattggaccacacaaagtcttactgatttcaggtaaaaacaataattgaagatgtccagcaaaacagcaagcaccaacaatatagcccaggcaaggagaactgtgcagcagttaagattagaagcctccattgaaagaataaaggtttcgaaggcatcagcggacctcatgtcctactgtgaggaacatgccaggagtgaccctttgctgataggaataccaacttcagaaaaccctttcaaggataaaaaaacttgcatcatcttatagtggaatagagaaacagctcctcgcctcttcccaacaacgcaaattatgagcagctccttgaagagatttaccttcagcttatttggtaaccactgctaataactaaaatgttctcagcttggaataatggactctgaagtctctattttccaagttgtcctttctcttaaaataccctttactgatttaatacagaataacaatcttattttccacttggtaactatggctttatgttgggttactgtttaaggaaagttgatctgggcctttttaaaaacataattatatactttagaaatacaagggattccgatatgtcaggacctaaatggcctaagcacctgtcaaattaaaattccaaaattcattgaaatcctaaagccttgatattatattctttataaggcgtgtgccagcctgtatagtatataagagagaggggtgtttgtgtgtatatatatatgcctttgtgtatacacttatcaaagctattttcttatgaaaacgtccctctctccataccatcagtttctcagttccagaagttatacctttattttgagctgtgtataggtagaataaaaaattcctttcatatcgttattgtacaaaaagtaaagagtatcctaaagattgtattcattgtaatcaagtaatgcaatcatctctcctctcttgaaatcttgctggacctcttaggctacaataaactgtaccaaactaaactgacagtccttcgataatatgaaacattaatttacaaggacccgttagggcttcaatgatgctgagtctggaaaaggggaggagacccttgggaggactccaggcagctgtgctcccagggctcatgttctctactggattagggataggcacctctgaaatctccaccctgatgattggaatgaaacggagtgaacatgagcccagctgagagaggagcaggaatgtgtgaaaaacagctgctctccttgtcccagctttgtgattgagccctctgtcttgctctctctgccttccctgtgtctttcttctcttaccagagcatctgtttgcagaggtaacattaccttccccagccacaggtatgtggaatacggtgaggaaaaaaaaaattaaaaaaaaaaatgcaagctgtcaggatgcttaagctcttttcagacatctgcagtttcattcctaccttgttcacataccatccaagaggcacataggctacccaagagagccttggattcagtggtacactccttgggcccaagggctttagcagctggatatggggttccttgattttcctctgggcccaaatatagccctcacactcttggaatttccaggtatgggggtagccccaaaaggaggaatctcctatggccaataaggtatcttgactttatcaaagtagaagagagggtcacttcggagtcaaatcatacactaggcctttgatgctttaattcttcttcagttcattaaaagtaactactaaggaaaggttaaaaacttcccctcaaaaaggaatcaaccccaggaagtaattatttacaacgattttcccaaattttgtacaatctgtcctggaaagcaaaccccttttaaaatctaatgtctgggctttgagtattagctcatttagggtggacaaatgcattactgttttcaaactgctcacatttattcagtatttctccaagttgctatctactcagccttatgaatgcccctcgcttttctaaggccatgtgaaaatcacggcactgcccttagccttgtgtcatctgctttttcgttctgcgatatgcccagttcccaaatcaattataggtacctgtttaggagagaggaagattttacctctcaaagggtgagatttgaaatttacactaaaaagacaactttacatttaatgcttcacttaatgagacattcttttttttataagtctatttttctactcagtttcagaacactaatctgattttcactctgatttttaacgtttctttaaatatttataatgtagcttctttcaaaatattttcatgaaaaattacttttattataccattatgtgcatgttattggtagcaggcatagtttattatttagtactgaaacatgctcttttacctaacagtaaacaagtatgttttgatatatatctgttaatatgcttatagtggtaagaaatggacttgaggtcccaggagatttcattttattcaccctggtcagatacaataaaggctatgagtataaatacataacttcctaaccaggtgtagggcatgttcatgaatatcaaatcttttgatgctggacccaagagaggaaaagttgtagctaaatgttgatttacttataactagacgtctatgtgagaaaatatatgtatacatatatatgatatgcagaagtcactttttttatcaggctttattctccttacaaagccacagtttaactgtctgcaacagttggtttatgttaatgatagacaaatacccagtgtttgttactttttccaactaccactgtaatgataatctttctcacgtatatacatgcaacttcttggcttcatttccatgaagctgtttcaatatattcagtatactttgtccttaatgctgcttctgttaacagtgatctctttctttttttcattcttatatcttcattagttcatcataaatctgtccagttgaggcctcaggaccacggcatgatttcatgactccgaagtattttacagaaacattttttaaataagggaaatattttatataccagatggttcacaagtgatggctcatagctagtttttttttttcttctaaaaaatgtcaggtttttaaaatcatttaccttattaaaatgaaaagtgccatacttaacttttaaaggaaagacctgacttgctttttctctatttagactgtttttgtactttactaatctttaaactatcaggaaaaaaaccaaaactttataccaatgatttagtaattttgaggcatagggtagcttacgtagtggaggatgtgccaaatattctcttcaaatgccaccttctcaatttataactaaaatagtgttatctgactaattcctctgaattttgatgtaagatctatataggcccccaaaatgatcgtagtacatgccagtcatttctcagtgaaataaatacaataccagagtacattatgggttttattgctttcttttatggtagacctgttaatggggaaaaaatacatcaaatcaaatagaatcttatatctgtatgttaaaatagagcacttacctgaagtcagtggcctggatcatagccctggatcatttcccagtctgtcctgtgctgtgtgaccttggacaaggcgcttcatctctctgggcctctatttctccatttgtaaaacaagtggctgcagtagatgatggctgagagcccttcctgttcccagatgccttggtccaaagaccccacccctctgctggtcctgccaacgtgttggtgctataagctgcttcagatataaaattggtttatctataatgtttgttcatttaatagcttctaaaaggcctttttgttatacagtgctttttttctagttttatggacttggttactgtaataatgtcttgtttttagccatgtaactacaaacagatattctcttgatgtcttagtaaatttgcatttgatatatcattgatgagattttgttgttatgtaatattctttggctacgcatctgtccagcatcttattaaccataatactgtgatcattatttggaaatatgtcctatggaaagaataaaagcatgtacttcacagctagcatgttcacagatttgaaagaagtttcattaaaagcaccattgctttctgtccaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaaaaaaaaaaaaaaaaa

SEQ ID NO: 6 - protein, 72 aa, Homo sapiens
MSSKTASTNNIAQARRTVQQLRLEASIERIKVSKASADLMSYCEEHARSDPLLIGIPTSENPFKDKKTCIIL

SEQ ID NO: 7 - DNA, 1739 bp, Homo sapiens
  CDS (157)..(1527)
ggggatcactgttggaaggcagctgcttgaggtccaaggcagtcagtgtcccctctcttttgcctcgggacagctggtatttatcagactcctaagaagttttccttgctccctagtagaagagagagattatgcagcgggcttttgattgatccaatgggaattacattgatctggtgtctggccttggttcttatcaagtggatcacctctaagaggcgtggagctatttcctatgacagttctgatcagactgcattatacattcgtatgctaggagatgtacgtgtaaggagccgagcaggatttgaatcagaaagaagaggttctcacccatatattgattttcgtattttccactctcaatctgaaattgaagtgtctgtctctgcaaggaatatcagaaggctactaagtttccagcgatatcttagatcttcacgcttttttcgtggtactgcggtttcaaattccctaaacattttagatgatgattataatggacaagccaagtgtatgctggaaaaagttggaaattggaattttgatatctttctatttgatagactaacaaatggaaatagtctagtaagcttaacctttcatttatttagtcttcatggattaattgagtacttccatttagatatgatgaaacttcgtagatttttagttatgattcaagaagattaccacagtcaaaatccttaccataacgcagtccacgctgcggatgttactcaggccatgcactgttacttaaaggaacctaagcttgccaattctgtaactccttgggatatcttgctgagcttaattgcagctgccactcatgatctggatcatccaggtgttaatcaacctttccttattaaaactaaccattacttggcaactttatacaagaatacctcagtactggaaaatcaccactggagatctgcagtgggcttattgagagaatcaggcttattctcacatctgccattagaaagcaggcaacaaatggagacacagataggtgctctgatactagccacagacatcagtcgccagaatgagtatctgtctttgtttaggtcccatttggatagaggtgatttatgcctagaagacaccagacacagacatttggttttacagatggctttgaaatgtgctgatatttgtaacccatgtcggacgtgggaattaagcaagcagtggagtgaaaaagtaacggaggaattcttccatcaaggagatatagaaaaaaaatatcatttgggtgtgagtccactttgcgatcgtcacactgaatctattgccaacatccagattggttttatgacttacctagtggagcctttatttacagaatgggccaggttttccaatacaaggctatcccagacaatgcttggacacgtggggctgaataaagccagctggaagggactgcagagagaacagtcgagcagtgaggacactgatgctgcatttgagttgaactcacagttattacctcaggaaaatcggttatcataacccccagaaccagtgggacaaactgcctcctggaggtttttagaaatgtgaaatggggtcttgaggtgagagaacttaactcttgactgccaaggtttccaagtgagtgatgccagccagcattatttatttccaagatttcctctgttggatcatttgaacccacttgttaattgcaagacccgaacatacagcaatatgaatttggcttt

SEQ ID NO: 8 - protein, 456 aa, Homo sapiens
MGITLIWCLALVLIKWITSKRRGAISYDSSDQTALYIRMLGDVRVRSRAGFESERRGSHPYIDFRIFHSQSEIEVSVSARNIRRLLSFQRYLRSSRFFRGTAVSNSLNILDDDYNGQAKCMLEKVGNWNFDIFLFDRLTNGNSLVSLTFHLFSLHGLIEYFHLDMMKLRRFLVMIQEDYHSQNPYHNAVHAADVTQAMHCYLKEPKLANSVTPWDILLSLIAAATHDLDHPGVNQPFLIKTNHYLATLYKNTSVLENHHWRSAVGLLRESGLFSHLPLESRQQMETQIGALILATDISRQNEYLSLFRSHLDRGDLCLEDTRHRHLVLQMALKCADICNPCRTWELSKQWSEKVTEEFFHQGDIEKKYHLGVSPLCDRHTESIANIQIGFMTYLVEPLFTEWARFSNTRLSQTMLGHVGLNKASWKGLQREQSSSEDTDAAFELNSQLLPQENRLS

SEQ ID NO: 9 - DNA, 5629 bp, Homo sapiens
  CDS (35)..(3742)
cttaagtgcaaggaactctgtgttgggaggaaaaatgtccttcttcaatttccgtaagatcttcaagttggggagcgagaagaagaagaagcagtacgaacacgtgaagagggacctgaaccccgaagacttttgggagattataggagaactgggcgacggagcctttgggaaagtgtacaaggcccagaataaagagaccagtgttttagctgctgcaaaagtgattgacactaaatctgaagaagaacttgaagattacatggtagagattgacatattagcatcttgtgatcacccaaatatagtcaagcttctagatgccttctattatgagaacaatctttggatcctcattgaattttgtgcaggtggagcagtagatgctgtgatgcttgaacttgagagaccattaactgagtcccaaatacaagtagtttgcaagcagactttagatgcattgaactacttacatgataataagatcatccacagagatctgaaggctggcaacattctctttaccttagatggagatatcaaattggcggattttggagtatcagctaaaaacacgaggacaattcaaagaagagattcctttattggtacaccatattggatggctcctgaagtagtcatgtgtgaaacatctaaggacagaccctatgactacaaagctgatgtttggtccctgggtatcactttaatagaaatggctgagatagaaccacctcatcatgaattaaatccaatgcgagtgctgctaaaaatagcaaaatctgagccacctacattagcacagccatccagatggtcttcaaattttaaggactttctaaagaaatgcttagaaaagaatgtggatgccaggtggactacatctcagctgctgcagcatccctttgttactgttgattccaacaaacccatccgagaattgattgcagaggcgaaggctgaagtaacagaagaagttgaagatggcaaagaggaagatgaagaggaggaaacagaaaattctctgccaatacctgcaagtaagcgtgcatcttctgaccttagtatcgccagctctgaagaagataaactttcacaaaatgcttgtattttggagtctgtctcagaaaaaacagaacgtagtaactctgaagataaactcaacagcaaaattcttaatgaaaaacccaccactgatgaacctgaaaaggctgtggaggatattaatgaacatattaccgatgctcagttagaagcaatgactgaactccatgacagaacagcagtaatcaaggagaatgaaagagagaagaggcccaagcttgaaaatctgcctgacacagaagaccaagaaactgtggacattaattcagtcagtgaaggaaaagagaataatataatgataaccttagaaacaaatattgaacataatctaaaatctgaggaagaaaaggatcaggaaaagcaacagatgtttgaaaataagcttataaaatctgaagaaattaaagatactattttgcaaacagtagatttagtttctcaagagactggagaaaaagaggcaaatattcaggcagttgatagtgaagttgggcttacaaaggaagacacccaagagaaattgggggaagacgacaaaactcaaaaagatgtgatcagcaatacaagtgatgtgataggaacatgtgaggcagcagatgtggctcagaaagtggatgaagacagtgctgaggatacgcagagtaatgatgggaaagaagtggtcgaagtaggccagaaattaattaataagcccatggtgggtcctgaggctggtggtactaaggaagttcctattaaagaaatagttgaaatgaatgaaatagaagaaggtaaaaataaggaacaagcaataaacagttcagagaacataatggacatcaatgaggaaccaggaacaactgaaggtgaagaaatcactgagtcaagtagcactgaagaaatggaggtcagaagtgtggtggctgatactgaccaaaaggctttaggaagtgaagttcaggatgcttctaaagtcactactcagatagataaagagaaaaaagaaattccagtgtcaattaaaaaagagcctgaagttactgtagtttcacagcccactgaacctcagcctgttctaatacccagtattaatatcaactctgacagtggagaaaataaagaagaaataggttctttatcaaaaactgaaactattctgccaccagaatctgagaatccaaaggaaaatgataatgattcaggcactggttccactgctgatactagcagtattgacttgaatttatccatctctagctttctaagtaaaactaaagacagtggatcgatatctttacaagaaacaagaagacaaaagaaaacattgaagaaaacacgcaaatttattgttgatggtgtagaagtgagtgtaacaacatcaaagatagttacagatagtgattccaaaactgaagaattgcggtttcttagacgtcaggaacttcgggaattaagatttcttcagaaagaagagcaaagagcccaacaacagctcaatagcaaactacagcaacaacgagaacaaattttccggcgctttgagcaggaaatgatgagtaaaaagcgacaatatgaccaggaaattgagaatctagaaaaacagcagaaacagactatcgaacgcctggaacaagagcacacaaatcgcttgcgagatgaagccaaacgcatcaaaggagaacaagagaaagagttgtccaaatttcagaatatgctgaagaaccgaaagaaggaggttataaatgaagtggagaaagcacccaaagagctgagaaaagagctcatgaaacgcaggaaagaggagcttgcacaaagccagcatgctcaggaacaagagtttgttcagaaacaacagcaagaattagatggctctctgaaaaagatcatccagcagcagaaggcagagttagctaatattgagagagagtgcctgaataacaagcaacagctcatgagagctcgagaagctgcaatttgggagctcgaagaacgacacttacaagaaaaacaccagctgctcaaacagcagcttaaagatcagtatttcatgcaaagacatcagctacttaagcgccacgagaaggaaacagagcaaatgcagcgttacaatcaaagacttattgaggaattgaaaaacagacagactcaagaaagagcaagactgcccaagattcagcgcagtgaagccaagactcgaatggccatgtttaagaagagtttgagaattaactcaacagccacaccagatcaggaccgtgataaaattaaacagtttgctgcacaagaagaaaagaggcagaaaaatgagagaatggctcagcatcagaaacatgagaatcaaatgcgagatcttcagttgcagtgtgaagccaatgtccgcgaactgcatcagctgcagaatgaaaaatgccacttgttggttgagcatgagactcagaaactgaaggagttagatgaggaacatagccaagaattaaaggagtggagagagaaattgagacctaggaaaaagacactggaagaagagtttgccaggaaactacaggaacaggaagtattctttaaaatgactggggagtctgaatgccttaacccatcaacacagagccggatttccaaattttatcctattcccagcttgcattccaccggatcataacaaagggaagcattctgtgcgtgggtttggctctttcagtatgtcattctgttctcatcttctgccacagtctctcagatagctcatgaagacaatcacctgcctcaccttctaggtgttttccttttttgttttttttgttttgttttgtttttaagcaaagatgaagggaaaacgaactaagacagacgctaggccatgttggcaaagtagcatcttggtgactaaggtgactttgtatattcatcttaaaaattatgttctttagacactgctacctgaaaactgttggagaaataatgtttaaagttatttaagaaaaactgttacatcactaagtattaataaattcttcttacctgacgtaacttctcaatgcctaaattctgtagttgaagctctgctgcagagagttgggataattttcttttggtggatcagctctcataaaaaagctatgatttgctcaaatatgctgttgactcagtaaatgaatatatttttttctttaaataggaacaacctcttttaaaagagaaaaattatttcagtgatttgtcaaaacgaattacctcttttggcatgagctaataattgagggtgctaattttcttaagatagtgcctaaaacactaaatttcagtcaagtcgtaagtaggattttctttttgatcaacagggacaaaaacatctttagaattaaaaacatggttgttttggaatttttgcttctcttaccgtttgatagaaattttcatcctaaaatacatgtacaaagtttggaaagatgaaaaaaagaggtagcttttagattgcaaattggaaatgtaaaactcatgaaatttaagcaatataggtttagctatctgtgtttattttctaaaataatacctgagctggttaaatgatttctctccatcttagctaattctgtttaaaactctgtcagaggcctgcaggctgtgagttatatttataaatatatcttcagaaattaatcttaaaagaggcattagttcagaatacttttttaaaagtttaaattaaatatttaggcacgtcagaaattacttttccttattttgaaatgaggctacttatgtcttggttttattttgttccatgtttaaatcattcactttgatttgagtgggaaaagcctgaagcctttatcatgtggttgctggtgtgtgtaattattaatgaaatgttcactcctagtcccttatgaggcttagaatttcaaccacgtgtcaggtcagacagtattataaactgtactttgctgtctgagacagcacatttgtgaatgatgcttgctgcctgccattttcaacctattctctcttaagagtgctaggtaccaaattgtgaaagtttgttttcagttatattacttttgaggctggtgaaaaatttaaatgtaactttgtgggaacactgattcatatttagaaaatgtaaatgtctgtagcactttcttgcagttaatttgaaaactttggatgctgaaccttgtttgtcagtgatttagatgatttaaaaatgcatgtgtgatttgaattttataattgttttgacaagcataatttacttggacaacttcgtaggtagccttaacttctggccaagtttgttttttatataaatatatatacatatatacatattatgtatggttgtaaattcatacacttatcacatgaatgtgttactgtatacaaaactcttaatgctttattctcaaatgctgggttgaaaaatgttttgaaagccttttaaaatatatatctttataaagtaatattcaggatgatgataaaaattgtttatattgttatgataaaaatgacagtataatgttaaaaaaaaaaaaaaaaaaaaaaa

SEQ ID NO: 10 - protein, 1235 aa, Homo sapiens
MSFFNFRKIFKLGSEKKKKQYEHVKRDLNPEDFWEIIGELGDGAFGKVYKAQNKETSVLAAAKVIDTKSEEELEDYMVEIDILASCDHPNIVKLLDAFYYENNLWILIEFCAGGAVDAVMLELERPLTESQIQVVCKQTLDALNYLHDNKIIHRDLKAGNILFTLDGDIKLADFGVSAKNTRTIQRRDSFIGTPYWMAPEVVMCETSKDRPYDYKADVWSLGITLIEMAEIEPPHHELNPMRVLLKIAKSEPPTLAQPSRWSSNFKDFLKKCLEKNVDARWTTSQLLQHPFVTVDSNKPIRELIAEAKAEVTEEVEDGKEEDEEEETENSLPIPASKRASSDLSIASSEEDKLSQNACILESVSEKTERSNSEDKLNSKILNEKPTTDEPEKAVEDINEHITDAQLEAMTELHDRTAVIKENEREKRPKLENLPDTEDQETVDINSVSEGKENNIMITLETNIEHNLKSEEEKDQEKQQMFENKLIKSEEIKDTILQTVDLVSQETGEKEANIQAVDSEVGLTKEDTQEKLGEDDKTQKDVISNTSDVIGTCEAADVAQKVDEDSAEDTQSNDGKEVVEVGQKLINKPMVGPEAGGTKEVPIKEIVEMNEIEEGKNKEQAINSSENIMDINEEPGTTEGEEITESSSTEEMEVRSVVADTDQKALGSEVQDASKVTTQIDKEKKEIPVSIKKEPEVTVVSQPTEPQPVLIPSININSDSGENKEEIGSLSKTETILPPESENPKENDNDSGTGSTADTSSIDLNLSISSFLSKTKDSGSISLQETRRQKKTLKKTRKFIVDGVEVSVTTSKIVTDSDSKTEELRFLRRQELRELRFLQKEEQRAQQQLNSKLQQQREQIFRRFEQEMMSKKRQYDQEIENLEKQQKQTIERLEQEHTNRLRDEAKRIKGEQEKELSKFQNMLKNRKKEVINEVEKAPKELRKELMKRRKEELAQSQHAQEQEFVQKQQQELDGSLKKIIQQQKAELANIERECLNNKQQLMRAREAAIWELEERHLQEKHQLLKQQLKDQYFMQRHQLLKRHEKETEQMQRYNQRLIEELKNRQTQERARLPKIQRSEAKTRMAMFKKSLRINSTATPDQDRDKIKQFAAQEEKRQKNERMAQHQKHENQMRDLQLQCEANVRELHQLQNEKCHLLVEHETQKLKELDEEHSQELKEWREKLRPRKKTLEEEFARKLQEQEVFFKMTGESECLNPSTQSRISKFYPIPSLHSTGS

SEQ ID NO: 11 - DNA, 4373 bp, Homo sapiens
  CDS (321)..(2861)
ggcgccgacccatgctggctgggaacgtgtctcccggtgacgcagccccgggtggggaacgtggtgcggcggcggcggcggcggcgactgtacgcgcctccgccgcccccgagaggacgcgccgtgcagcggctgagtggcggcggcggcgacggcaaacccggagctgccggccggcgcgcgggaggaggacgcgggtgcggtctaggaaacggagctgcgggcggaggctccatgttgggaagcggcgccgttcgtgcttgttagcgggaatccgggagccgcggggtgagctggcgggggccgggccctaagtgaagatggaggccccgctgcggcctgccgcggacatcctgaggcggaacccgcagcaggactacgaactcgtccagagggtcggcagcggcacctacggggacgtctataaggccagaaatgtacacacaggagagctggctgcagtaaaaatcattaaattggagcctggagatgatttttctttgattcaacaagaaatatttatggttaaagaatgtaaacattgtaacatcgttgcctactttgggagttatcttagtcgggaaaaactatggatttgtatggaatactgtggtggcggatcacttcaagatatttaccatgttactggaccattatcagaattgcaaatagcctatgtatgcagagaaaccttacagggtcttgcctatttgcatactaaaggcaaaatgcatagagatatcaaaggtgctaatattttattgacagaccatggcgatgtaaaattagctgactttggtgtggctgcaaaaataacagctaccattgcaaaacgaaaatctttcattggcaccccttactggatggccccagaagttgcagcagtagagaagaatggtggctacaaccaactctgtgatatctgggcagtaggaataacagcaattgaacttggagaacttcagccacctatgtttgatctccacccaatgagggctctcttcttaatgtcaaaaagtaattttcagcctccaaaactaaaggacaaaacaaaatggtcatcaacattccataattttgtcaaaatagcactaaccaaaaacccaaaaaaaagaccaactgctgaaagacttctgactcacacttttgttgcacagccaggtctctctagagccctagcagttgaactgttagacaaagtgaacaatccagataaccacgcacattacactgaagcagatgacgatgactttgagccccatgcaatcattcgtcataccattagatctacaaacaggaatgccagagctgaacggacagcttcagaaataaattttgacaaattacaatttgaacctcctctgagaaaagaaacagaagcacgagatgaaatgggattgtcatcagacccaaatttcatgttacagtggaatccttttgttgatggtgcaaatactggcaaatcaacctcaaaacgtgcaataccacctcccctacctcctaagccaaggataagcagttaccctgaagacaactttccggatgaagaaaaagcatcaaccataaaacattgtcctgattcagaaagcagagctccccaaattctcagaagacagagtagcccaagttgtgggcctgtggcagagacttcttctattggaaatggtgatggtatttcaaaactgatgagtgaaaatacagaaggatcagcacaagcaccacagttaccacgaaaaaaggacaaacgagacttccctaaaccagccatcaatggccttccacccaccccaaaagttctgatgggagcatgcttttcaaaagtttttgatggctgtcctttgaaaattaattgtgcaacatcctggatacatcctgatacaaaagatcagtacattatttttggaactgaagatggtatttacacactgaatctcaatgagctacatgaggcaacgatggaacagttatttccacggaagtgtacttggctgtatgttatcaataatactttaatgtcattatcagaaggaaaaacctttcagctctactctcacaatcttatagctttgtttgaacatgccaaaaaaccaggattagctgcccatattcaaactcacaggtttccagaccgaatactaccaagaaaattcgctttaacaacaaagattcctgatacaaaaggctgccacaaatgttgcatagtcagaaacccttacacgggacataaatacctctgtggagctttacagtctggaattgttttacttcagtggtatgagccaatgcagaaattcatgttgataaagcactttgattttcctttgccaagtcctttgaatgtttttgaaatgctggtgatacctgaacaggaataccctatggtctgtgtagctattagcaaaggcactgaatcgaatcaggtagttcagtttgagacaatcaatttgaactctgcatcttcatggtttacagaaattggtgcaggcagccagcagttagattccattcatgtaacacagttggagagagataccgttttagtgtgtttagacaaatttgtgaaaattgtaaatctacaaggaaaattaaaatcaagtaagaaactggcctctgagttaagttttgattttcgcattgaatctgtagtatgccttcaagacagtgtgttggctttctggaaacatgggatgcagggtaaaagcttcaagtcagatgaggttacccaggagatttcagatgaaacaagagttttccgcttattaggatcagacagggttgtcgttttggaaagtaggccaacagaaaatcctactgcacacagcaatctctacatcttggctggacatgaaaatagttactaagcaacagaaactgatctcaaatgacaggaaaatgaatatactccattgaaaggaaaaataaggaaattcaatacaaactgcactatgatttgctttaactattatgggttatattgcaaatgatctgtactttagggtagaattcaatattttctgcagctggaaacagctagtctatctcttgccactgtgtggtggttatatcaagtttgcttaataaaagctatgagacaaatagtcctctagttccaggaaacacagtctttttttaaaaaaaacaatgtttgtaacaagggtgccatggtatttttagataactcgtgattatcttaagagaggtaaatttagtgatcattttatatcatgtcttattccttcttaatgaacataatttgttaaattctcaagcaaggttttcacttttatattggccattctgtatgtttttgtaaaacagaatatttaatccttatttattaatctcttgctggagtggtgtaatgtatctaacttttagcaaaggagggttgcagagcagcttaaattttttttataatgtataagaattttgtttatcttttaagagtagtaaagtactttgagtgtttgggggttcaacacacacatgcaattttgcttaacaaaagtattttataatacagtttcatacagaattaccttaaaagggagtcttatgttttcaactacagatagttgtaagggatcatacagaagatattgatgatagttgaaatattcttagaaggggtgtgtatgtctagctgtgtctaccatgtgtatgtattcttgacaagcagtataaaatacctgtgatttttctttacattagggataatgcataaggaattaatcttcatatatattatcatccctaatgtagcagggggaagtatttaattgcccatgatatgtattttacttatactatgccagagaggaaactataaagtaattacacatgtaatcttgggtttttcacatatgtaggtattcattttgagtaggttgaagaagaaaaaaaatatttaaatgaattgaattcctgatgggatagtatcaataagtatttaaaagccagtattctaaaaataataaagggtagggtcatttttgagtttgtttttcttttgctattgttaatattcaaaattaaagtgttacattggtacctgttgtcttaatgcatttattgagaacagcattgagatgatgaacaaggggttagcaatagcaaactctataattattttgactaattacttaagaggaaaacagtataagtatctcattcagtatttagcaattctgtaaaataagtattatctctatttttcagatgaggaagtaagggtttagcaaggttaagagatctatccaatttacacagcaagttagtagttgagcctgaccatgagtcttctgactctgttcttttcactatgcaatacgcaaacaataaaatgttatacaaataaaaaaaaaaaaaaaaaa

SEQ ID NO: 12 - protein, 846 aa, Homo sapiens
MEAPLRPAADILRRNPQQDYELVQRVGSGTYGDVYKARNVHTGELAAVKIIKLEPGDDFSLIQQEIFMVKECKHCNIVAYFGSYLSREKLWICMEYCGGGSLQDIYHVTGPLSELQIAYVCRETLQGLAYLHTKGKMHRDIKGANILLTDHGDVKLADFGVAAKITATIAKRKSFIGTPYWMAPEVAAVEKNGGYNQLCDIWAVGITAIELGELQPPMFDLHPMRALFLMSKSNFQPPKLKDKTKWSSTFHNFVKIALTKNPKKRPTAERLLTHTFVAQPGLSRALAVELLDKVNNPDNHAHYTEADDDDFEPHAIIRHTIRSTNRNARAERTASEINFDKLQFEPPLRKETEARDEMGLSSDPNFMLQWNPFVDGANTGKSTSKRAIPPPLPPKPRISSYPEDNFPDEEKASTIKHCPDSESRAPQILRRQSSPSCGPVAETSSIGNGDGISKLMSENTEGSAQAPQLPRKKDKRDFPKPAINGLPPTPKVLMGACFSKVFDGCPLKINCATSWIHPDTKDQYIIFGTEDGIYTLNLNELHEATMEQLFPRKCTWLYVINNTLMSLSEGKTFQLYSHNLIALFEHAKKPGLAAHIQTHRFPDRILPRKFALTTKIPDTKGCHKCCIVRNPYTGHKYLCGALQSGIVLLQWYEPMQKFMLIKHFDFPLPSPLNVFEMLVIPEQEYPMVCVAISKGTESNQVVQFETINLNSASSWFTEIGAGSQQLDSIHVTQLERDTVLVCLDKFVKIVNLQGKLKSSKKLASELSFDFRIESVVCLQDSVLAFWKHGMQGKSFKSDEVTQEISDETRVFRLLGSDRVVVLESRPTENPTAHSNLYILAGHENSY

SEQ ID NO: 13 - DNA, 4421 bp, Homo sapiens
  CDS (369)..(2909)
ggcgccgacccatgctggctgggaacgtgtctcccggtgacgcagccccgggtggggaacgtggtgcggcggcggcggcggcggcgactgtacgcgcctccgccgcccccgagaggacgcgccgtgcagcggctgagtggcggcggcggcgacggcaaacccggagctgccggccggcgcgcgggaggaggacgcgggtgcggtctaggaagtaggtcccgggctccgcgctcaacaaagacggggccgcccccattagacggagctgcgggcggaggctccatgttgggaagcggcgccgttcgtgcttgttagcgggaatccgggagccgcggggtgagctggcgggggccgggccctaagtgaagatggaggccccgctgcggcctgccgcggacatcctgaggcggaacccgcagcaggactacgaactcgtccagagggtcggcagcggcacctacggggacgtctataaggccagaaatgtacacacaggagagctggctgcagtaaaaatcattaaattggagcctggagatgatttttctttgattcaacaagaaatatttatggttaaagaatgtaaacattgtaacatcgttgcctactttgggagttatcttagtcgggaaaaactatggatttgtatggaatactgtggtggcggatcacttcaagatatttaccatgttactggaccattatcagaattgcaaatagcctatgtatgcagagaaaccttacagggtcttgcctatttgcatactaaaggcaaaatgcatagagatatcaaaggtgctaatattttattgacagaccatggcgatgtaaaattagctgactttggtgtggctgcaaaaataacagctaccattgcaaaacgaaaatctttcattggcaccccttactggatggccccagaagttgcagcagtagagaagaatggtggctacaaccaactctgtgatatctgggcagtaggaataacagcaattgaacttggagaacttcagccacctatgtttgatctccacccaatgagggctctcttcttaatgtcaaaaagtaattttcagcctccaaaactaaaggacaaaacaaaatggtcatcaacattccataattttgtcaaaatagcactaaccaaaaacccaaaaaaaagaccaactgctgaaagacttctgactcacacttttgttgcacagccaggtctctctagagccctagcagttgaactgttagacaaagtgaacaatccagataaccacgcacattacactgaagcagatgacgatgactttgagccccatgcaatcattcgtcataccattagatctacaaacaggaatgccagagctgaacggacagcttcagaaataaattttgacaaattacaatttgaacctcctctgagaaaagaaacagaagcacgagatgaaatgggattgtcatcagacccaaatttcatgttacagtggaatccttttgttgatggtgcaaatactggcaaatcaacctcaaaacgtgcaataccacctcccctacctcctaagccaaggataagcagttaccctgaagacaactttccggatgaagaaaaagcatcaaccataaaacattgtcctgattcagaaagcagagctccccaaattctcagaagacagagtagcccaagttgtgggcctgtggcagagacttcttctattggaaatggtgatggtatttcaaaactgatgagtgaaaatacagaaggatcagcacaagcaccacagttaccacgaaaaaaggacaaacgagacttccctaaaccagccatcaatggccttccacccaccccaaaagttctgatgggagcatgcttttcaaaagtttttgatggctgtcctttgaaaattaattgtgcaacatcctggatacatcctgatacaaaagatcagtacattatttttggaactgaagatggtatttacacactgaatctcaatgagctacatgaggcaacgatggaacagttatttccacggaagtgtacttggctgtatgttatcaataatactttaatgtcattatcagaaggaaaaacctttcagctctactctcacaatcttatagctttgtttgaacatgccaaaaaaccaggattagctgcccatattcaaactcacaggtttccagaccgaatactaccaagaaaattcgctttaacaacaaagattcctgatacaaaaggctgccacaaatgttgcatagtcagaaacccttacacgggacataaatacctctgtggagctttacagtctggaattgttttacttcagtggtatgagccaatgcagaaattcatgttgataaagcactttgattttcctttgccaagtcctttgaatgtttttgaaatgctggtgatacctgaacaggaataccctatggtctgtgtagctattagcaaaggcactgaatcgaatcaggtagttcagtttgagacaatcaatttgaactctgcatcttcatggtttacagaaattggtgcaggcagccagcagttagattccattcatgtaacacagttggagagagataccgttttagtgtgtttagacaaatttgtgaaaattgtaaatctacaaggaaaattaaaatcaagtaagaaactggcctctgagttaagttttgattttcgcattgaatctgtagtatgccttcaagacagtgtgttggctttctggaaacatgggatgcagggtaaaagcttcaagtcagatgaggttacccaggagatttcagatgaaacaagagttttccgcttattaggatcagacagggttgtcgttttggaaagtaggccaacagaaaatcctactgcacacagcaatctctacatcttggctggacatgaaaatagttactaagcaacagaaactgatctcaaatgacaggaaaatgaatatactccattgaaaggaaaaataaggaaattcaatacaaactgcactatgatttgctttaactattatgggttatattgcaaatgatctgtactttagggtagaattcaatattttctgcagctggaaacagctagtctatctcttgccactgtgtggtggttatatcaagtttgcttaataaaagctatgagacaaatagtcctctagttccaggaaacacagtctttttttaaaaaaaacaatgtttgtaacaagggtgccatggtatttttagataactcgtgattatcttaagagaggtaaatttagtgatcattttatatcatgtcttattccttcttaatgaacataatttgttaaattctcaagcaaggttttcacttttatattggccattctgtatgtttttgtaaaacagaatatttaatccttatttattaatctcttgctggagtggtgtaatgtatctaacttttagcaaaggagggttgcagagcagcttaaattttttttataatgtataagaattttgtttatcttttaagagtagtaaagtactttgagtgtttgggggttcaacacacacatgcaattttgcttaacaaaagtattttataatacagtttcatacagaattaccttaaaagggagtcttatgttttcaactacagatagttgtaagggatcatacagaagatattgatgatagttgaaatattcttagaaggggtgtgtatgtctagctgtgtctaccatgtgtatgtattcttgacaagcagtataaaatacctgtgatttttctttacattagggataatgcataaggaattaatcttcatatatattatcatccctaatgtagcagggggaagtatttaattgcccatgatatgtattttacttatactatgccagagaggaaactataaagtaattacacatgtaatcttgggtttttcacatatgtaggtattcattttgagtaggttgaagaagaaaaaaaatatttaaatgaattgaattcctgatgggatagtatcaataagtatttaaaagccagtattctaaaaataataaagggtagggtcatttttgagtttgtttttcttttgctattgttaatattcaaaattaaagtgttacattggtacctgttgtcttaatgcatttattgagaacagcattgagatgatgaacaaggggttagcaatagcaaactctataattattttgactaattacttaagaggaaaacagtataagtatctcattcagtatttagcaattctgtaaaataagtattatctctatttttcagatgaggaagtaagggtttagcaaggttaagagatctatccaatttacacagcaagttagtagttgagcctgaccatgagtcttctgactctgttcttttcactatgcaatacgcaaacaataaaatgttatacaaataaaaaaaaaaaaaaaaaa

SEQ ID NO: 14 - protein, 846 aa, Homo sapiens; identical to SEQ ID NO: 12

SEQ ID NO: 15 - DNA, 1767 bp, Homo sapiens
  CDS (236)..(1480)
cagcggcggtggcccaggtagaggggtccgcgctggcggcggcggcggcgctgttccccgcgcggtccgcggagcggggtccgggctgcgcgacgtggggcggcggcggcactgcggccccggcccaagcccgaccccgggtcccctcctcggccgccccccgcccggccgcccgccctcgggcctccccccgggccctcggtcccctcccccgctggcggggcccggacagaagatggtgcagaagaaaccagccgaacttcagggtttccaccgttcgttcaaggggcagaaccccttcgagctggccttctccctagaccagcccgaccacggagactctgactttggcctgcagtgctcagcccgccctgacatgcccgccagccagcccattgacatcccggacgccaagaagaggggcaagaagaagaagcgcggccgggccaccgacagcttctcgggcaggtttgaagacgtctaccagctgcaggaagatgtgctgggggagggcgctcatgcccgagtgcagacctgcatcaacctgatcaccagccaggagtacgccgtcaagatcattgagaagcagccaggccacattcggagcagggttttcagggaggtggagatgctgtaccagtgccagggacacaggaacgtcctagagctgattgagttcttcgaggaggaggaccgcttctacctggtgtttgagaagatgcggggaggctccatcctgagccacatccacaagcgccggcacttcaacgagctggaggccagcgtggtggtgcaggacgtggccagcgccttggactttctgcataacaaaggcatcgcccacagggacctaaagccggaaaacatcctctgtgagcaccccaaccaggtctcccccgtgaagatctgtgacttcgacctgggcagcggcatcaaactcaacggggactgctcccctatctccaccccggagctgctcactccgtgcggctcggcggagtacatggccccggaggtagtggaggccttcagcgaggaggctagcatctacgacaagcgctgcgacctgtggagcctgggcgtcatcttgtatatcctactcagcggctacccgcccttcgtgggccgctgtggcagcgactgcggctgggaccgcggcgaggcctgccctgcctgccagaacatgctgtttgagagcatccaggagggcaagtacgagttccccgacaaggactgggcccacatctcctgcgctgccaaagacctcatctccaagctgctggtccgtgacgccaagcagaggctgagtgccgcccaagtcctgcagcacccctgggttcaggggtgcgccccggagaacaccttgcccactcccatggtcctgcagaggtgggacagtcacttcctcctccctccccacccctgtcgcatccacgtgcgacctggaggactggtcagaaccgttactgtgaatgagtgaagatcctggaggaccctgggccccaggccagctcccatcgctgggggacggtgaacggccatgtgttaatgttacgatgtttttaaaagacaaaaaaaaaaaaaaaacctcaaaagtttttttaaagtgggggaaaaacatccaagcactttaattccaatgtaccaggtgaactgacggagctcagaagttttcctttacaccaactgtcaatgccggaattttgtattctgttttgtaaagatttaataaaagtcaaaaaacttgcaaaaaaaaaaaaaaaaaaa

SEQ ID NO: 16 - protein, 414 aa, Homo sapiens
MVQKKPAELQGFHRSFKGQNPFELAFSLDQPDHGDSDFGLQCSARPDMPASQPIDIPDAKKRGKKKKRGRATDSFSGRFEDVYQLQEDVLGEGAHARVQTCINLITSQEYAVKIIEKQPGHIRSRVFREVEMLYQCQGHRNVLELIEFFEEEDRFYLVFEKMRGGSILSHIHKRRHFNELEASVVVQDVASALDFLHNKGIAHRDLKPENILCEHPNQVSPVKICDFDLGSGIKLNGDCSPISTPELLTPCGSAEYMAPEVVEAFSEEASIYDKRCDLWSLGVILYILLSGYPPFVGRCGSDCGWDRGEACPACQNMLFESIQEGKYEFPDKDWAHISCAAKDLISKLLVRDAKQRLSAAQVLQHPWVQGCAPENTLPTPMVLQRWDSHFLLPPHPCRIHVRPGGLVRTVTVNE

SEQ ID NO: 17 - DNA, 3795 bp, Homo sapiens
  CDS (236)..(1633)
cagcggcggtggcccaggtagaggggtccgcgctggcggcggcggcggcgctgttccccgcgcggtccgcggagcggggtccgggctgcgcgacgtggggcggcggcggcactgcggccccggcccaagcccgaccccgggtcccctcctcggccgccccccgcccggccgcccgccctcgggcctccccccgggccctcggtcccctcccccgctggcggggcccggacagaagatggtgcagaagaaaccagccgaacttcagggtttccaccgttcgttcaaggggcagaaccccttcgagctggccttctccctagaccagcccgaccacggagactctgactttggcctgcagtgctcagcccgccctgacatgcccgccagccagcccattgacatcccggacgccaagaagaggggcaagaagaagaagcgcggccgggccaccgacagcttctcgggcaggtttgaagacgtctaccagctgcaggaagatgtgctgggggagggcgctcatgcccgagtgcagacctgcatcaacctgatcaccagccaggagtacgccgtcaagatcattgagaagcagccaggccacattcggagcagggttttcagggaggtggagatgctgtaccagtgccagggacacaggaacgtcctagagctgattgagttcttcgaggaggaggaccgcttctacctggtgtttgagaagatgcggggaggctccatcctgagccacatccacaagcgccggcacttcaacgagctggaggccagcgtggtggtgcaggacgtggccagcgccttggactttctgcataacaaaggcatcgcccacagggacctaaagccggaaaacatcctctgtgagcaccccaaccaggtctcccccgtgaagatctgtgacttcgacctgggcagcggcatcaaactcaacggggactgctcccctatctccaccccggagctgctcactccgtgcggctcggcggagtacatggccccggaggtagtggaggccttcagcgaggaggctagcatctacgacaagcgctgcgacctgtggagcctgggcgtcatcttgtatatcctactcagcggctacccgcccttcgtgggccgctgtggcagcgactgcggctgggaccgcggcgaggcctgccctgcctgccagaacatgctgtttgagagcatccaggagggcaagtacgagttccccgacaaggactgggcccacatctcctgcgctgccaaagacctcatctccaagctgctggtccgtgacgccaagcagaggctgagtgccgcccaagtcctgcagcacccctgggttcaggggtgcgccccggagaacaccttgcccactcccatggtcctgcagaggaacagctgtgccaaagacctcacgtccttcgcggctgaggccattgccatgaaccggcagctggcccagcacgacgaggacctggctgaggaggaggccgcggggcagggccagcccgtcctggtccgagctacctcacgctgcctgcagctgtctccaccctcccagtccaagctggcgcagcggcggcaaagggccagtctgtcctcggccccagtggtcctggtgggagaccacgcctgaccctcccatctcccctctgtacataggtcacccgtcccccaatcaaatctaaaggttttttaagctatcgccagccggtgtccagcgggctgcccctcctctgcctggattcccaggcactaagctcagctgaggggggtgttttatagaaggtttttgcttttgggttttttttttcctgtttccacccctccccgttattttttcctttggatggttaaaagcattgcaggcacccgggaaggtgagcagagggtaggtgggtgggcttgtcccctccccggtcccccgccctgctcacctctactatgaaggtgcccccaggtcacctgtgctgcccgccatctgcccacgtggcttgcagtgactcaggagagcaggcccacagcgtttgccatcttgcagagctggggaggggcacaggaccctgccctcgtgttccctcccagcccgcagtatttcagggacaggctcttcccctctatccctcaccctgagagcacccctggtggcttggttggggaagggaggggctgcctgtctctggaggtgtcaggcaggcaggtggcaggcagctcacccacccaccccatgggatcccccagcccttcacccgcgcctgccttgtccccatgatagttgacaatcggggcttcctgcaaggcccgtctgtctgtccaggactcctggtggccagattcggcctccgaccttgaccttaaactgcagctgaccccaggggctcgccgctgcccctcccctccacaccaaggcctgagacagcaggagccccgcctggcccgaagccgtttccaccgcagcaggcagaggggctggacaggcactgtcagccaatgtggggggtcctgaagacacccccttggggcacccgagtgccccttctcagggctcagtctgaccgtagccacgtcctgcctcgcgccgcccctcgggcctgacctggaagctccgtcagctccgtccttgtccttagagctgagcccagaccccggggtctggccgaatcctcacccccagggcagtgtttttggtctgccaccttcaggaaaacggctgcggcctcggcctcccttcgggcacccaggaatgcgggggtctgctcagtccccccaccctccatgctccaacccccgggggctgcggagcctgctgccccctccccgcgggtggggacgttctatgcaatacagggttccactttagaagtgcgcgcggctagggtcaccgcccgcccttcccggcgcagcccccgagctccacagctggggcagcccctctggcttctaaatccgcggtcgggattcttcctcctgtttagttttttagtttttccttaaaaaaaaacaacacatcgatggactttgcttccctgttcttgaagaatacttgaatgtcggggggcctgggggtgggggcctcggagaccgtctgcctggccctgctgcccctcctgaatctcgtatgatggtcacagtccggtggccgtgggggtgctctgccttccctggtccccactgcccatatctgtggactgccccttccaaagacccctggggggggtggggcattccgcccacccctttcccccatcacttctcgcctgtcagtgattccatgtttcgtaacgggggattctctgcctttttgtatcaaagaacaagcaaatggacccccgcccgctgcaggcgcccatagccatcgggtctctaaagctgagtggctagcagcgtttgtttgtttgtttttttttttttttctgaaggtgggacagtcacttcctcctccctccccacccctgtcgcatccacgtgcgacctggaggactggtcagaaccgttactgtgaatgagtgaagatcctggaggaccctgggccccaggccagctcccatcgctgggggacggtgaacggccatgtgttaatgttacgatgtttttaaaagacaaaaaaaaaaaaaaaacctcaaaagtttttttaaagtgggggaaaaacatccaagcactttaattccaatgtaccaggtgaactgacggagctcagaagttttcctttacaccaactgtcaatgccggaattttgtattctgttttgtaaagatttaataaaagtcaaaaaacttgcaaaaaaaaaaaaaaaaaaaa

SEQ ID NO: 18 - protein, 465 aa, Homo sapiens
MVQKKPAELQGFHRSFKGQNPFELAFSLDQPDHGDSDFGLQCSARPDMPASQPIDIPDAKKRGKKKKRGRATDSFSGRFEDVYQLQEDVLGEGAHARVQTCINLITSQEYAVKIIEKQPGHIRSRVFREVEMLYQCQGHRNVLELIEFFEEEDRFYLVFEKMRGGSILSHIHKRRHFNELEASVVVQDVASALDFLHNKGIAHRDLKPENILCEHPNQVSPVKICDFDLGSGIKLNGDCSPISTPELLTPCGSAEYMAPEVVEAFSEEASIYDKRCDLWSLGVILYILLSGYPPFVGRCGSDCGWDRGEACPACQNMLFESIQEGKYEFPDKDWAHISCAAKDLISKLLVRDAKQRLSAAQVLQHPWVQGCAPENTLPTPMVLQRNSCAKDLTSFAAEAIAMNRQLAQHDEDLAEEEAAGQGQPVLVRATSRCLQLSPPSQSKLAQRRQRASLSSAPVVLVGDHA

SEQ ID NO: 19 - DNA, 3097 bp, Homo sapiens
  CDS (275)..(940)
agactatctttctagacaaggcagttgaggaggagggagcgcttgagggggactggcctggcgtgcactccgcacctcggggacattattgcgcgtggaacggctgcttttggaaggcacaacttcctgaatggaccatgactcccaccaaagatccctgtctctgattcaccaaacagcttcaaccctgaaaccaggacgagaagttgacaacatctgagtggacagctaattgacctaagacttcagaccagactattgcccagaagaaaagatgtttggttttcacaagccaaagatgtaccgaagtatagagggctgctgtatttgcagagctaagtcctccagttctcgattcactgacagtaaacgctatgaaaaggacttccagagctgttttggattgcatgagactcgttcaggagacatctgcaatgcctgtgtcctgcttgtgaaaagatggaagaagttgccagcaggatcaaaaaaaaactggaatcatgtggtagatgcaagggctggacccagtctaaagactacattgaaaccaaagaaagtgaaaactctatctgggaacaggataaaaagcaaccagatcagtaaactgcagaaggaatttaaacgtcataattctgatgctcacagtaccacctcaagtgcctccccagctcaatctccttgttacagtaaccagtcagatgacggctcagatacagagatggcttctggttctaacagaacaccagttttttcctttttagatctcacttactggaaaagacagaagatatgttgtgggatcatctataaaggccgttttggggaagtcctcattgacacacatctcttcaagccttgctgcagcaataagaaagcagctgctgagaagccagaggagcaggggccagagcctctgcccatctccactcaggagtggtgactgaggtttttatgtagaaggggaacaaaaaaaaaaatctgaattttgaaaaaccacaaagctacaaactgaccctctttttttttttttttttttgagacggagttttgctcttgttacccaggctggagtgcagtggcgtgatcttggctcactgcaacttccgtctcccgggttcaagtgattctcctgcctcagcctcccaagtagctgggtttataggtgcccgccaccagacccggctaattttttagttttagtagagacggggtttcaccacgttggccaggctggtcttaaatgaccctcttatttttaacttggatacctgctattctgccaaaagacaatttctagagtagttttgaatgggttgatttcccccactcccacaaactctgaagccagtgtctagcttactaaaaaaagagttgtatataatatttaagatgctgagtatttcataggaaagctgaatgctgctgtaaagtgctctttaagtctttttttttttaatccccttctaatgaatgaaactaggggaatttcaggggacagagatgggatttgttgtatgataaactgtatgtagtttttagtctttctgttttgagaagcagtggttggggcatttttaagatggctggctactcttgttttccctcatgataataaatttgtcataactcagtaacatgaacttgcccctagaggtagttgttaataattttgaaatattaaggtcttgccaagcttctgatgattcacacctgtactactgattattaagcaggacagactgagctttctgttgcaaataccttggaggagaaagtaatttctaaatatacagagaggtaacttgactatatatgttgcatcctgtgcctcccttcatattaatatttgataaagattttaatttatgtaaaacttctaaagcagaatcaaagctcctcttggggaaatggcaagtctttaggataggcaagaccctgtatgaatagtaccaaagcattaccgcatggtagagaacacactcgattaaaaatgttaagctatctgaaaaataaaatgtgcaagtcttcaggatggcacaaaacaaaggttaatgcttcttggggcacatttcttagagggcttgctgagtgtgtaaatataatcgacttttgtttgtgttacatgacttctgtgacttcattgaaaatctgcacaattcagtttcagctctggattacttcagttgacctttgtgaaggtttttatctgtgtagaatgggtgtttgacttgttttagcctattaaatttttattttctttcactctgtattaaaagtaaaacttactaaaagaaaagaggtttgtgttcacattaaatggttttggtttggcttcttttagtcaggctttctgaacattgagatatcctgaacttagagctcttcaatcctaagattttcatgaaaagcctctcacttgaacccaaaccagagtactcttactgcctcttttctaaatgttcaggaaaagcattgccagttcagtcttttcaaaatgagggagaaacatttgcctgccttgtaataacaagactcagtgcttattttttaaactgcattttaaaaattggatagtataataacaataaggagtaagccaccttttataggcaccctgtagttttatagttcttaatctaaacattttatatttccttcttttggaaaaaacctacatgctacaagccaccatatgcacagactatacagtgagttgagttggctctcccacagtctttgaggtgaattacaaaagtccagccattatcatcctcctgagttatttgaaatgattttttttgtacattttggctgcagtattggtggtagaatatactataatatggatcatctctacttctgtatttatttatttattactagacctcaaccacagtcttctttttccccttccacctctctttgcctgtaggatgtactgtatgtagtcatgcactttgtattaatatattagaaatctacagatctgttttgtactttttatactgttggatacttataatcaaaacttttactagggtattgaataaatctagtcttactagaaaaaaaaaaaaaagaaaaaaaaaaaaaaaaa

SEQ ID NO: 20 - protein, 221 aa, Homo sapiens
MFGFHKPKMYRSIEGCCICRAKSSSSRFTDSKRYEKDFQSCFGLHETRSGDICNACVLLVKRWKKLPAGSKKNWNHVVDARAGPSLKTTLKPKKVKTLSGNRIKSNQISKLQKEFKRHNSDAHSTTSSASPAQSPCYSNQSDDGSDTEMASGSNRTPVFSFLDLTYWKRQKICCGIIYKGRFGEVLIDTHLFKPCCSNKKAAAEKPEEQGPEPLPISTQEW

SEQ ID NO: 21 - DNA, 2156 bp, Homo sapiens
  CDS (69)..(419)
ggaggccagtgcgcggccgcggtgctctaccggcgtgtcgctccgccccagggagagccggcgctaccatggaggagtaccatcgccactgcgacgaggttggcttcaatgctgaggaagcccacaatattgtcaaagagtgtgtagatggggttttaggtggtgaagattataatcacaacaacatcaaccagtggactgcaagcatagtggaacaatccttaacacacctggttaagttgggaaaagcctataaatatattgtgacctgtgcagtggtccagaagagcgcatatggctttcacacagccagctcctgtttttgggataccacatctgatggaacctgtaccgtaagatgggagaaccggaccatgaactgtattgtcaacgtttttgccattgctattgttctttaactgactaaaaatgttgggctaaagccattaacttaagaatttgtcagtgtatcctttccaaaaagagtaatagttgtttactagtgtgctagatgaaaagcgtgcaatatgctttaaagctatcaacaaaaactgaatattataagcaagcaatatcatagtaattggcagattagctcatattctatacagcatcgtttaaataggaaaaatttaatgctagcaaaaaataaatttagaatatggcatgacatgaaaatacaatcttatatttacaccagcttttcactaatattttgtacctaaggtgatggggaactccattcagataataaaattctctttcagctagagaagttaacaggaataaatatatgaacaaaaaagctgcaaggataaatgtggagaaaatgatgagaattagctaacatttttaagtttttttaaactttcttcccctcagttgtacttaatatttagtggaaagtaataatttttttattttctatcaactaatagtatagtaacaactatgattaacttgtttactttttctgaggattagtaaatcaattttttttaatttcaaattttggatttacacttgagggtaaattaaatctggtaaactgaatttcctagttaaataaaattagttgcagtatatgatgaacagtgtatgactcaaacagctgccttacaattcactcattccatgtggaacaaacatttatcagatgcctattatgggcatatgtctctgctaagcaccatagttgtcaatgtgctgtgcaaatgctaagttccttttagcaattgttcagttggaagacgtattaatatttggggaaggaaaagaaagtagttgttttacaagggaggaaaaaagtgaatctggttacacatatggaagtaagcaaaatgaaaagcacttattgctttctgacagaattatagatgtaattttaagagttgctcctagcaagttaaaagtgcatataaaatatgcaactcttagttaaaggccttattatcagtcttacctatacaagtagtaaattttgtcattgctttagttacaaccatctgtaaataacttaaaagacttattatgtggggttcaaattgagtggaataaagtatagattaaaagtatacaatccttagcacgttatctcagggcttatgaaatgtaattaaatttattaagaaaatagatgaaaaattagggtacacagctggccaccaaatgcgaagtcaatctgctacttaaccctgaaaacaaaatcagttttgcatattaccactaacactaatacatatagagagcggaaccataactcattgaattttggagaggaataagcttagcgttaatattgacaatattaaggcaatattcttgtaggaatactatgtgcatgtttgatattttgccaaataacaataattaataattgttcaatgtttaagaataatattaacaaaataaaggagtttaatgcagtgatctttgtttttggcacatcaaaaattctcagtcattattcatgtttcttttatgctgctggcttttgtgccctggaagatcataatagtgaccaaaatatacatgcagacttgttttttattattgttgtttaagcataatttaagaaaaaaaatttttacctggtgaacttgctatctgctctgtttctagttaaaatataataaatattatcttcctgtgctgta

SEQ ID NO: 22 - protein, 116 aa, Homo sapiens
MEEYHRHCDEVGFNAEEAHNIVKECVDGVLGGEDYNHNNINQWTASIVEQSLTHLVKLGKAYKYIVTCAVVQKSAYGFHTASSCFWDTTSDGTCTVRWENRTMNCIVNVFAIAIVL

SEQ ID NO: 23 - DNA, 5060 bp, Homo sapiens
  CDS (543)..(4619)
gcacgaggggagttcggcgtttgctggggctgcagcagctgaagtgtagtgttttcttgggactggcggtctgcacttctctcccgggttccatctccccccgcccggtggtgaggccctcgaggagggctcggacgggtgtagcgatccgcgctagaggaagacgaggcccgggaacgcatgtcccccagggcaggttagggggctggaggggtcaaatcccggggtacttgtggagactctttagcgtggcttcttctctctgctgagaccccgagagctttcccagttctcctcccaggaccaccggggttcctgaagatcgggacttttctgcgcccctccaccaacagcccatctcctgtctatgaagaaagacccttcgtagaaacaacttccccgctgctgacgcgttttcccgtcccgtccccgaagtagtctactatgacctcgttgtgagcctctgaacgattttgacactttcccgaggcctagggtattatatcctaaccttactaaagaccacagaggtgcttgccattatgggaaatcagcttgctggcattgctccctcccagatcctttctgtagagagttatttttcagatattcatgactttgaatatgataaaagcctggggagtactcggttttttaaagttgctcgagccaagcaccgagaaggcctggtcgttgtgaaggtttttgcaattcaggatcccacattgcctttaaccagctataaacaagagctggaggaactgaaaatcaggcttaattctgcacagaattgtctacctttccagaaagcatcagaaaaagcatctgagaaagcagctatgctctttaggcagtatgtgcgagacaatctctatgatcgcatcagtacccgtccattcttgaataacattgagaagcgctggattgctttccagatcctgacagctgtggaccaagcacacaaatctggagttcgtcatggggacatcaagactgagaatgtgatggtcaccagttggaattgggttcttctaactgattttgccagttttaagcccacttatcttccagaagacaacccggcagatttcaattatttctttgacacatcacggaggagaacttgctatattgctcctgaacgttttgttgatggtgggatgtttgccactgagttagaatatatgagagatccttcaactccgcttgtagacttaaatagcaatcagagaacaagaggagagttgaagagagcaatggacatcttttcagcaggttgtgtgatagctgagctttttacagaaggtgtaccattatttgatctctctcaacttttggcttatagaaatggacattttttccctgaacaagtgctaaataaaattgaagatcacagtatcagagaattggtaactcagatgattcaccgtgagccagataaacgtttagaggcagaagattacttaaaacagcagcgtggcaatgcctttcctgaaatattttacacttttcttcagccctacatggcccagtttgccaaggaaacgtttctttctgcagatgagcgtattctggttatacggaaggatttgggcaacattattcacaatctctgtggacatgatctgccagaaaaagccgaaggagagcctaaggaaaatgggctggttatcttggtatctgttataacatcctgcctacagacccttaaatactgtgattccaaactagctgctttggaactgattcttcatttggctccaagattaagtgttgaaatccttttggatcgtattactccatatcttttgcatttcagcaatgactctgttcctagggtgagggctgaagccttgaggacgttgaccaaagttcttgctctcgtcaaagaggttcctcgtaatgatatcaatatttatccggaatacattctgccaggcatagcccacttagcccaagatgatgctactatcgttagactagcctatgctgaaaacatagctctgctggcagaaacagctctgagattcctggaattagtacagttaaaaaatcttaatatggaaaatgaccccaataatgaagaaatagatgaggttacacatccaaatggaaattatgacacagagctccaagccttacatgaaatggtccagcagaaagttgttactttgctaagtgaccctgaaaatattgtaaaacaaaccttgatggaaaatggaataacacggctgtgtgtattctttggacgtcagaaagccaacgatgttttgttgtcccacatgattactttcctaaatgataagaatgattggcatctacgtggagcattttttgatagtatagttggtgttgctgcctatgttggctggcaaagctcctcaattctcaagcctctgctgcaacaaggtcttagtgatgctgaggaatttgtcattgtgaaagctctttatgcccttacttgtatgtgccagttaggactgctacaaaaaccccatgtttacgaatttgccagtgatattgcccccttcctgtgtcatcccaatttatggatacgttatggtgccgtgggatttatcacagtggtagctcgtcaaataagtacagctgatgtctactgtaaactgatgccttatcttgacccatatattacccaaccaataatacagattgaaagaaaacttgttctgctcagtgttttaaaggaaccagtaagtcgttctatatttgattatgctttgaggtctaaagatattactagcttgttcagacatcttcacatgcgtcagaagaaacgaaatggttctcttcccgactgccctccgccagaggatcctgccatagcacagcttctgaagaagttgctctcacagggaatgacagaggaagaggaagacaaacttctggcactgaaagacttcatgatgaaatctaataaagcaaaggccaatatagtggaccagagccatcttcatgatagtagtcagaaaggtgtaattgacttggcagctttaggcataactgggagacaagttgatcttgttaaaaccaaacaagaaccagatgacaaacgggccagaaaacatgtaaaacaagactcaaatgtaaatgaagaatggaaaagcatgtttgggtcactggacccaccaaacatgccacaggccctacctaaagggagtgatcaggaggtgattcagactgggaaacctcctcgttccgagtcctctgctggcatttgtgtccctttgtcaacttcttcacaggttccagaagtgacaactgtccaaaataaaaaaccagtaataccggttttaagtagtacaatcttaccatccacctatcagattcgaattacaacttgtaaaactgaacttcagcaactcatccagcaaaagcgggagcagtgcaatgctgagagaatagctaagcagatgatggaaaatgctgaatgggagagtaaaccaccaccacctggatggcgtcctaaagggctgttagttgcccatcttcatgagcataaatctgctgtgaatcgaattagagtctctgatgaacactcactttttgcaacatgttcaaatgatggcacagtgaaaatctggaacagtcaaaagatggaggggaagaccaccactaccagatctattcttacatacagccgaattggaggacgagtcaagacgctcacattctgccaaggctcccactatttagccatagcatctgataatggtgctgtccagcttcttggaattgaggcttctaagctgcccaagtctcctaaaatccatcctctacaaagcagaattctagatcagaaggaggacggttgtgttgtggatatgcatcacttcaactctggagcacagtctgttcttgcctatgccactgtgaatggctctctggttggctgggaccttaggtcttcaagcaatgcgtggactttaaagcatgatttaaagtcgggcctcatcacttcctttgctgtggacatccaccaatgctggctctgcattggtacaagcagtggtaccatggcttgttgggacatgaggttccagttgccaatttcaagtcactgtcatccttccagggctcgaatcagacgcctctcaatgcaccctctgtatcagtcctgggtgattgcagctgttcagggcaacaacgaagtgtccatgtgggacatggagactggtgacagaagatttactctctgggccagcagtgcaccaccactttctgaattacagccttctcctcatagcgtccatggtatctactgtagtcctgcagatggaaatcctatcctactaacagctggctcagatatgaaaataaggttttgggacttggcttacccagaaaggtcctatgttgttgcaggaagtactagttccccatctgtgtcctactacaggaaaataattgaaggcactgaagttgtccaggaaattcagaataagcagaaagtaggaccaagtgatgacacccctcgaaggggcccagagtccctgcccgtgggacatcatgacatcatcactgatgtcgccacattccagaccacacagggcttcatcgtaactgcttctagagatgggattgtgaaggtgtggaaataaaacctactgatttgtataaattttaatagttataaatataatactataactcgagaaaaggcatttctagagaacagattcatttgcttaattttcaaaattatgtctccatattactgtttcatgactgactgactaaatgacacccaaaatggttaagatgtacttgactagtttacttatgcatctctttgcaagaatcagccagccaacaatgtctgggatttttattgtatatgttatagaggtgagaaatgtaaaatatgaaaatgaatatgtttattttgtattgaaaaagatggttgaaaagatggttgtaagctattatagtataaacacatttttgctattaaaaatgctattcaaagcagttaaactgtaaaaaaaaaaaaaaaaaaaaaaaaaaaaaaaaaaaaaaaactcgagggggggcccggtacc

SEQ ID NO: 24 - protein, 1358 aa, Homo sapiens
MGNQLAGIAPSQILSVESYFSDIHDFEYDKSLGSTRFFKVARAKHREGLVVVKVFAIQDPTLPLTSYKQELEELKIRLNSAQNCLPFQKASEKASEKAAMLFRQYVRDNLYDRISTRPFLNNIEKRWIAFQILTAVDQAHKSGVRHGDIKTENVMVTSWNWVLLTDFASFKPTYLPEDNPADFNYFFDTSRRRTCYIAPERFVDGGMFATELEYMRDPSTPLVDLNSNQRTRGELKRAMDIFSAGCVIAELFTEGVPLFDLSQLLAYRNGHFFPEQVLNKIEDHSIRELVTQMIHREPDKRLEAEDYLKQQRGNAFPEIFYTFLQPYMAQFAKETFLSADERILVIRKDLGNIIHNLCGHDLPEKAEGEPKENGLVILVSVITSCLQTLKYCDSKLAALELILHLAPRLSVEILLDRITPYLLHFSNDSVPRVRAEALRTLTKVLALVKEVPRNDINIYPEYILPGIAHLAQDDATIVRLAYAENIALLAETALRFLELVQLKNLNMENDPNNEEIDEVTHPNGNYDTELQALHEMVQQKVVTLLSDPENIVKQTLMENGITRLCVFFGRQKANDVLLSHMITFLNDKNDWHLRGAFFDSIVGVAAYVGWQSSSILKPLLQQGLSDAEEFVIVKALYALTCMCQLGLLQKPHVYEFASDIAPFLCHPNLWIRYGAVGFITVVARQISTADVYCKLMPYLDPYITQPIIQIERKLVLLSVLKEPVSRSIFDYALRSKDITSLFRHLHMRQKKRNGSLPDCPPPEDPAIAQLLKKLLSQGMTEEEEDKLLALKDFMMKSNKAKANIVDQSHLHDSSQKGVIDLAALGITGRQVDLVKTKQEPDDKRARKHVKQDSNVNEEWKSMFGSLDPPNMPQALPKGSDQEVIQTGKPPRSESSAGICVPLSTSSQVPEVTTVQNKKPVIPVLSSTILPSTYQIRITTCKTELQQLIQQKREQCNAERIAKQMMENAEWESKPPPPGWRPKGLLVAHLHEHKSAVNRIRVSDEHSLFATCSNDGTVKIWNSQKMEGKTTTTRSILTYSRIGGRVKTLTFCQGSHYLAIASDNGAVQLLGIEASKLPKSPKIHPLQSRILDQKEDGCVVDMHHFNSGAQSVLAYATVNGSLVGWDLRSSSNAWTLKHDLKSGLITSFAVDIHQCWLCIGTSSGTMACWDMRFQLPISSHCHPSRARIRRLSMHPLYQSWVIAAVQGNNEVSMWDMETGDRRFTLWASSAPPLSELQPSPHSVHGIYCSPADGNPILLTAGSDMKIRFWDLAYPERSYVVAGSTSSPSVSYYRKIIEGTEVVQEIQNKQKVGPSDDTPRRGPESLPVGHHDIITDVATFQTTQGFIVTASRDGIVKVWK

SEQ ID NO: 25 - DNA, 1646 bp, Homo sapiens
  CDS (412)..(1323)
gctgttgcgggagcgcgccctcagcgaagcaagtgaggcatctcactgggaaagtcgaatgtgtgtggcggccgccgccgaggcgggttccgaagagacctcagcagggcaggccagggcctacgcgaacgcccacccttaagagcgcggggacagggaactggagcgttcctcccagcccccgacgtcgcgggcccagtgtccccgtccaggctggttgggcgcacgcgcggccccactcgcccccacgcgtgcgtccccgctggtcccgcccccggccggaagttccggcggcggagctgggccgggcccgagcggatcgcgggctcgggctgcggggctccggctgcgggcgctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaagcccccggcccctatacatagtttatgttcctggccaccccgaccgcttcccccaagttctgctgttggactctggactgtttcccctctcagcatcgcttttgctgggccgtgatcgtccccctttgtgggctggaaaagcaggtgagggtgggctgggctgaggccattgccgccactatctgtgtaataaaatccgtgagcacgaggtgggacgtgctggtgtgtgaaaaaaaaaaaaaaaaaaaaaaaaaaaaaaaaaaaaaaaaaaaaa

SEQ ID NO: 26 - protein, 303 aa, Homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 27 - DNA, 1946 bp, Homo sapiens
  CDS (114)..(1541)
gcttctgagcggaaactgaaagtgaaatagggagctggctaccagcgttgagtcccctgtaaagccaaaccccctaaaggtctccacactgctgtttaacggcacacttgacaatggcttcagcagcacgcttgacaatgatgtgggaggaggtcacatgccctatctgcctggaccccttcgtggagcctgtgagcatcgagtgtggccacagcttctgccaggaatgcatctctcaggttgggaaaggtgggggcagcgtctgtcctgtgtgccggcagcgctttctgctcaagaatctccggcccaatcgacagctagccaacatggtgaacaaccttaaagaaatcagccaggaggccagagagggcacacagggggaacggtgtgcagtgcatggagagagacttcacctgttctgtgagaaagatgggaaggccctttgctgggtatgtgcccagtctcggaaacaccgtgaccacgccatggtccctcttgaggaggctgcacaggagtaccaggagaagctccaggtggcattaggggaactgagaagaaagcaggagttggctgagaagttggaagtggaaattgcaataaagagagcagactggaagaaaacagtggaaacacagaaatctaggattcacgcagagtttgtgcagcaaaaaaacttcctggttgaagaagaacagaggcagctgcaggagctggagaaggatgagagggagcagctgagaatcctgggggagaaagaggccaagctggcccagcagagccaggccctacaggagctcatctcagagctagatcgaaggtgccacagctcagcactggaactgctgcaggaggtgataattgtcctggaaaggagtgagtcctggaacctgaaggacctggatattacctctccagaactcaggagtgtgtgccatgtgccagggctgaagaagatgctgaggacatgtgcagtccacatcactctggatccagacacagccaatccgtggctgatactttcagaagatcggagacaagtgaggcttggagacacccagcagagcatacctggaaatgaagagagatttgatagttatcctatggtcctgggtgcccagcactttcactctggaaaacattactgggaggtagatgtgacaggaaaggaggcctgggacctgggtgtctgcagagactctgtgcgcaggaaggggcactttttgcttagttccaagagtggcttctggacaatttggttgtggaacaaacaaaaatatgaggctggcacctacccccagactcccctccaccttcaggtgcctccatgccaagttgggattttcctggactatgaggctggcatggtctccttctacaacatcactgaccatggctccctcatctactccttctctgaatgtgcctttacaggacctctgcggcccttcttcagtcctggtttcaatgatggaggaaaaaacacagcccctctaaccctctgtccactgaatattggatcacaaggatccactgactattgatggctttctctggacactgccactctccccattggcaccgcttctcagccacaaaccctgcctcttttccccatgaactctgaaccacctttgtctctgcagaggcatccggatcccagcaagcgagctttagcagggaagtcacttcaccatcaacattcctgccccagatggctttgtgattccctccagtgaagcagcctccttatatttggcccaaactcatcttgatcaaccaaaaacatgtttctgccttctttatgggacttaagttttttttttctcctctccatctctaggatgtcgtctttggtgagatctctattatatcttgtatggtttgcaaaagggcttcctaaaaataaaaaataaaatttaaaaaactgtgaaaaaaaaaaaaaaaaa

SEQ ID NO: 28 - protein, 475 aa, Homo sapiens [sequence omitted]

SEQ ID NO: 29 - DNA, 3507 bp, Homo sapiens
  CDS (101)..(3406)
ccacgcgtccgagcggggcgcctgggtggatgggcgcttgggcgcctgggctgccggacggtgggaacggaagtcgctgtgggacgctgaggaagccaggatggcgactccgagcaagaagacgtcaactccaagcccccagccttccaagagagctctcccgagagacccttcgtcggaggtcccgagcaagaggaagaattcggccccgcagctgccgctgttgcagtcgtccgggcctttcgtggaaggctctatcgtccgcatctcgatggagaacttcctaacatatgatatttgtgaagtatctcctggaccccacttgaatatgatcgttggagccaatggaacagggaagtcgagcattgtgtgtgccatttgccttggtttagctggaaaacctgctttcatgggacgagcagataaggttgggttttttgtgaagagaggatgttctagaggcatggttgaaattgaattgttcagggcttctggaaatcttgtaatcacccgtgagattgatgtggcaaaaaatcagtccttttggttcatcaacaaaaaatctacaacccagaaaatagtggaagagaaagttgcagccttaaatattcaagtggggaatctttgccagtttctccctcaggacaaagttggagaatttgctaaactcagcaaaattgaactcctcgaagccactgaaaagtcaattggtcccccagaaatgcacaaatatcactgtgaactcaaaaacttaagggagaaagaaaaacagctcgagacctcatgcaaagagaaaactgagtatctacagaaaatggttcagaggaatgaaagatataaacaagatgtggagaggttctatgaacggaagcgacatttagatttaattgagatgcttgaagcaaaaaggccatgggtggaatatgaaaatgttcgtcaggaatatgaagaagtaaaactagttcgtgaccgagtgaaggaagaggtcagaaaacttaaagaagggcagattcctataacatgtcgaattgaagaaatggaaaacgagcgtcacaatttggaggctcgaatcaaagaaaaggcaacagatattaaggaggcatctcaaaaatgcaaacagaagcaagatgttatagaaaggaaagataaacatattgaggaacttcagcaggctttaatagtaaagcaaaatgaagagcttgaccgacagaggagaataggtaatacccgcaaaatgatagaggatttgcaaaatgaactaaagaccacggaaaactgcgagaatcttcagccccagattgatgccattacaaatgatctgagacggattcaggatgaaaaggcattatgtgaaggcgaaataattgataagcgaagagagagggaaactctagagaaggagaaaaagagtgtggacgatcatattgtacgttttgacaatcttatgaatcagaaggaagataagctaagacagagattccgtgacacgtatgatgctgttttatggctaagaaataacagagacaaatttaaacaaagagtctgtgagcccataatgctcacgatcaatatgaaagataataaaaatgccaaatatattgaaaatcatattccatcaaatgacttaagagcctttgtatttgaaagtcaagaagatatggaggttttcctcaaagaggttcgtgacaataaaaaattaagagtaaatgctgttattgctcccaagagttcatatgcagacaaagcaccttcaagatctttgaatgaacttaaacaatacggatttttctcttatttgagagaattatttgatgcacctgatcctgtaatgagttacctttgctgtcagtatcatattcatgaagttcctgtaggaactgaaaagaccagagaaagaattgaacgggtaatacaagaaacccgattaaaacagatttatacagcagaagaaaagtatgtggtgaaaacttctttttattcaaacaaagttatttctagtaacacatctctaaaagtagcgcagtttctcactgtcactgtggacctagagcagagaagacacttagaagaacagctaaaggaaattcatagaaaattgcaagcagtggattcagggttgattgccttacgtgaaacaagcaaacatctggagcacaaagacaatgaacttagacaaaagaagaaggagcttcttgagagaaaaaccaagaaaagacaactggaacaaaaaatcagttccaaactaggaagtttaaagctgatggaacaggatacttgcaatcttgaagaggaagagcgaaaagcaagtaccaaaatcaaagaaataaatgttcaaaaagcgaaacttgttaccgaattaacaaacctaataaagatttgtacttctttgcatatacaaaaagtagatttaattctccaaaatactacagtgatctctgagaagaacaaattagaatcagattatatggccgcatcttcacaactccgtcttacagagcaacatttcattgaattggatgaaaatagacagagattattgcagaaatgcaaggaacttatgaaaagagctaggcaagtatgtaacctgggtgcagagcagactcttcctcaagaataccagacacaagtacccaccattccaaatggacacaactcctcactccccatggttttccaagaccttccaaacacattggatgaaattgatgctttattaactgaagaaagatcaagagcttcctgcttcacgggactgaatcctacaattgttcaggaatatacaaaaagagaagaagaaatagaacagttaactgaggaactaaagggaaagaaagttgaactagatcaatacagggaaaacatttcacaggtaaaagaaaggtggcttaatcctttaaaagagctggtagaaaaaattaatgaaaaattcagcaatttttttagttccatgcagtgtgctggtgaagttgatctccatacagaaaatgaggaagattatgataaatatggaattcgaattagagtcaaatttcgaagtagtactcaactgcatgaattaactcctcatcatcaaagtggaggtgaaagaagtgtttctaccatgttatacttgatggcacttcaggagctaaatagatgtccattcagagtagttgatgaaatcaatcagggaatggacccaatcaatgaacggagagtgtttgaaatggttgtaaatactgcctgtaaagaaaatacatctcaatactttttcataacaccaaagctcctgcaaaatcttccttattctgaaaagatgacagttttgtttgtctacaatggccctcatatgctggaaccaaacacatggaatttaaaggctttccaaaggcggcggcgccgtattacattcactcaaccttcttaataaaagtaaagagagggaacttgggaattttttttgttaaattctgtttataagtatggctcaactgaataaaaggagattcactaaaaaaaaaaaaaaaa

SEQ ID NO: 30 - protein, 1101 aa, Homo sapiens
MATPSKKTSTPSPQPSKRALPRDPSSEVPSKRKNSAPQLPLLQSSGPFVEGSIVRISMENFLTYDICEVSPGPHLNMIVGANGTGKSSIVCAICLGLAGKPAFMGRADKVGFFVKRGCSRGMVEIELFRASGNLVITREIDVAKNQSFWFINKKSTTQKIVEEKVAALNIQVGNLCQFLPQDKVGEFAKLSKIELLEATEKSIGPPEMHKYHCELKNLREKEKQLETSCKEKTEYLQKMVQRNERYKQDVERFYERKRHLDLIEMLEAKRPWVEYENVRQEYEEVKLVRDRVKEEVRKLKEGQIPITCRIEEMENERHNLEARIKEKATDIKEASQKCKQKQDVIERKDKHIEELQQALIVKQNEELDRQRRIGNTRKMIEDLQNELKTTENCENLQPQIDAITNDLRRIQDEKALCEGEIIDKRRERETLEKEKKSVDDHIVRFDNLMNQKEDKLRQRFRDTYDAVLWLRNNRDKFKQRVCEPIMLTINMKDNKNAKYIENHIPSNDLRAFVFESQEDMEVFLKEVRDNKKLRVNAVIAPKSSYADKAPSRSLNELKQYGFFSYLRELFDAPDPVMSYLCCQYHIHEVPVGTEKTRERIERVIQETRLKQIYTAEEKYVVKTSFYSNKVISSNTSLKVAQFLTVTVDLEQRRHLEEQLKEIHRKLQAVDSGLIALRETSKHLEHKDNELRQKKKELLERKTKKRQLEQKISSKLGSLKLMEQDTCNLEEEERKASTKIKEINVQKAKLVTELTNLIKICTSLHIQKVDLILQNTTVISEKNKLESDYMAASSQLRLTEQHFIELDENRQRLLQKCKELMKRARQVCNLGAEQTLPQEYQTQVPTIPNGHNSSLPMVFQDLPNTLDEIDALLTEERSRASCFTGLNPTIVQEYTKREEEIEQLTEELKGKKVELDQYRENISQVKERWLNPLKELVEKINEKFSNFFSSMQCAGEVDLHTENEEDYDKYGIRIRVKFRSSTQLHELTPHHQSGGERSVSTMLYLMALQELNRCPFRVVDEINQGMDPINERRVFEMVVNTACKENTSQYFFITPKLLQNLPYSEKMTVLFVYNGPHMLEPNTWNLKAFQRRRRRITFTQPS

SEQ ID NO: 31 - DNA, 1720 bp, Homo sapiens
  CDS (196)..(1467)
agaagggcaacctcgtgctttctgcagaggagaccggagggcagaaggcagagtccaggcttagactgcagttcctcgcttacctgtgcagtctaattttgagctgcctctttgtagtcttaaaaggcaggagcttcgtgttgtgggtctgctaacccgtacgtttccgtgggcaagtcgtgtgtactcctcgccatggctcagctccaaacacgcttctacactgataacaagaaatatgccgtagatgatgttcccttctcaatccctgctgcctctgaaattgccgaccttagtaacatcatcaataaactactaaaggacaaaaatgagttccacaaacatgtggagtttgatttccttattaagggccagtttctgcgaatgcccttggacaaacacatggaagtggagaacatctcatcagaagaagttgtggaaatagaatacgtggagaagtgtaccgcaccccagccagagcaatgcatgttccatgatgactggatcagttcaattaaaggggcagaggaatggatcttgactggttcttatgataagacttctcggatctggtccttggaaggaaagtcaataatgacaattgtgggacatacggatgttgtaaaagatgtggcctgggtgaaaaaagatagtttgtcctgcttattattgagtgcttctatggatcagactattctcttatgggagtggaatgtagagagaaacaaagtgaaagccctacactgctgtagaggtcatgctggaagtgtagattctatagctgttgatggctcaggaactaaattttgcagtggctcctgggataagatgctaaagatctggtctacagtccctacagatgaagaagatgaaatggaggagtccacaaatcgaccaagaaagaaacagaagacagaacagttgggactaacaaggactcccatagtgaccctctctggccacatggaggcagtttcctcagttctgtggtcagatgctgaagaaatctgcagtgcatcttgggaccatacaattagagtgtgggatgttgggtctggcagtcttaagtcaactttgacaggaaataaagtgtttaattgtatttcctattctccactttgtaaacgtttagcatctggaagcacagataggcatatcagactgtgggatccccgaactaaagatggttctttggtgtcgctgtccctaacgtcacatactggttgggtgacatcagtaaaatggtctcctacccatgaacagcagctgatttcaggatctttagataacattgttaagctgtgggatacaagaagttgtaaggctcctctctatgatctggctgctcatgaagacaaagttctgagtgtagactggacagacacagggctacttctgagtggaggagcagacaataaattgtattcctacagatattcacctaccacttcccatgttggggcatgaaagtgaacaataatttgactatagagattatttctgtaaatgaaattggtagagaaccatgaaattacatagatgcagatgcagaaagcagccttttgaagtttatataatgttttcacccttcataacagctaacgtatcactttttcttattttgtatttataataagataggttgtgtttatgaaatacaaactgtggcatacattctctatacaaacttgaaattaaactgagttttacatttctcttt

SEQ ID NO: 32 - protein, 423 aa, Homo sapiens
MAQLQTRFYTDNKKYAVDDVPFSIPAASEIADLSNIINKLLKDKNEFHKHVEFDFLIKGQFLRMPLDKHMEVENISSEEVVEIEYVEKCTAPQPEQCMFHDDWISSIKGAEEWILTGSYDKTSRIWSLEGKSIMTIVGHTDVVKDVAWVKKDSLSCLLLSASMDQTILLWEWNVERNKVKALHCCRGHAGSVDSIAVDGSGTKFCSGSWDKMLKIWSTVPTDEEDEMEESTNRPRKKQKTEQLGLTRTPIVTLSGHMEAVSSVLWSDAEEICSASWDHTIRVWDVGSGSLKSTLTGNKVFNCISYSPLCKRLASGSTDRHIRLWDPRTKDGSLVSLSLTSHTGWVTSVKWSPTHEQQLISGSLDNIVKLWDTRSCKAPLYDLAAHEDKVLSVDWTDTGLLLSGGADNKLYSYRYSPTTSHVGA

SEQ ID NO: 33 - DNA, 2834 bp, Homo sapiens
  CDS (51)..(2708)
gtctcctcggccgacaagctctcgcgagacgagccgtgcaggctgaaaaaatggcgccacccagtacccgggagcccagggtcctgtcggcgaccagcgcaaccaaatccgacggagagatggtgctgccaggcttcccggacgccgacagctttgtgaagtttgctcttgggtccgtggtggcagtcaccaaggcatctgggggcctaccacagtttggcgatgagtatgatttttaccgaagttttcctggcttccaagcattttgcgaaacacagggagacaggttgcttcagtgcatgagcagagtaatgcagtaccatgggtgtcgcagcaacattaaggatcgaagtaaagtgactgagctggaagacaagtttgatttactagttgatgccaatgatgtaattctggagagagtgggtattttactggatgaagcctcaggtgtaaacaagaatcaacagcctgtcctccctgccggcttgcaggtccccaaaacggtagtgtccagctggaaccgtaaggcagcagaatatggcaaaaaagcaaaatctgaaactttccggctgcttcatgcaaaaaatatcatccgacctcagctcaagtttcgagagaagattgacaattccaacacaccatttcttcctaaaatcttcatcaaacccaatgctcagaaacctctccctcaagctctctctaaggaaaggcgggaacgcccacaggatcgtcctgaggacttggacgtcccccctgcactggctgatttcatccatcagcagagaacccagcaggttgagcaagacatgtttgcacatccttatcaatatgaactaaatcactttaccccagcagatgcagtgcttcaaaagccacaaccccagttatacagacctatagaagagacaccatgccatttcatatcctccctggatgaactcgtggaactcaacgaaaagctcttgaattgtcaggaatttgcagttgacttggagcaccactcttacaggagcttcctgggactgacctgcctgatgcaaatttctactcggacggaagacttcatcattgacaccctcgagcttcgaagtgacatgtacattctcaatgagagcctcacagacccagccatcgttaaggtctttcatggtgctgattcagacatagaatggctacagaaagactttgggttgtatgtagtaaacatgtttgatactcatcaggcagcacgccttcttaacctgggcaggcactcactcgatcatctcctgaaactctactgcaacgtggactcaaacaagcaatatcagctggctgattggagaatacgccctctgcccgaggagatgctcagctacgcccgggatgacacccattacctgctatatatctatgacaaaatgaggctggagatgtgggagcgcggcaacgggcagccggtgcagctgcaggtggtgtggcaacggagcagggacatctgcctcaagaaattcatcaaacctatcttcacggatgagtcctaccttgaactctataggaagcagaagaagcaccttaacacacagcagttgacagcctttcagctgctgtttgcctggagggataaaacagctcgcagggaagatgaaagttacggatatgtactgccaaaccacatgatgctgaaaatagctgaagaactgcctaaggaacctcagggcatcatagcttgctgcaacccagtaccgccccttgtgcggcagcagatcaacgaaatgcaccttttaatccagcaggcccgagagatgcccctgctcaagtctgaagttgcagccggagtgaagaagagcggaccgctgcccagtgctgagagattggagaatgttctctttggacctcacgactgctcccatgcccctccggatggctatccaatcatcccaaccagtggatctgtgccagttcagaagcaggcgagcctcttccctgatgaaaaagaagataacttgctgggtaccacatgcctgattgccacagctgtcatcacgttatttaatgaacctagtgctgaagacagtaaaaagggtccattgacagttgcacagaaaaaagcccagaacatcatggagtcctttgaaaatccatttaggatgtttctgccctcactgggacaccgtgctcccgtctctcaggcagcgaagttcgatccatcaaccaaaatctatgaaatcagcaaccgctggaagctggcccaggtacaagtacaaaaagactctaaagaagctgtcaagaagaaggcagctgagcaaacagctgcccgggaacaggcaaaggaggcgtgcaaagctgcagcagaacaggccatctccgtccgacagcaggtcgtgctagaaaatgctgcaaagaagagagagcgagcaacaagcgacccaaggaccacagaacagaaacaagagaagaaacgactcaaaatttccaagaagccaaaggacccagagccaccagaaaaagagtttacgccttacgactacagccagtcagacttcaaggcttttgctggaaacagcaaatccaaagtttcttctcagtttgatccaaataaacagaccccgtctggcaagaaatgcattgcagccaaaaaaattaaacagtcggtgggaaacaaaagcatgtcctttccaactggaaagtcagacagaggcttcaggtacaactggccacagagatagtcctggaagacacgtggcgcctgtggaccggaagcaccaaatgctggtgctgcttttgtacatacatatttttaaaccattaaaattcttcctgaagaaaaaaaaaaaaaaaaaaaaaaaaaaaaa

SEQ ID NO: 34 - protein, 885 aa, Homo sapiens
MAPPSTREPRVLSATSATKSDGEMVLPGFPDADSFVKFALGSVVAVTKASGGLPQFGDEYDFYRSFPGFQAFCETQGDRLLQCMSRVMQYHGCRSNIKDRSKVTELEDKFDLLVDANDVILERVGILLDEASGVNKNQQPVLPAGLQVPKTVVSSWNRKAAEYGKKAKSETFRLLHAKNIIRPQLKFREKIDNSNTPFLPKIFIKPNAQKPLPQALSKERRERPQDRPEDLDVPPALADFIHQQRTQQVEQDMFAHPYQYELNHFTPADAVLQKPQPQLYRPIEETPCHFISSLDELVELNEKLLNCQEFAVDLEHHSYRSFLGLTCLMQISTRTEDFIIDTLELRSDMYILNESLTDPAIVKVFHGADSDIEWLQKDFGLYVVNMFDTHQAARLLNLGRHSLDHLLKLYCNVDSNKQYQLADWRIRPLPEEMLSYARDDTHYLLYIYDKMRLEMWERGNGQPVQLQVVWQRSRDICLKKFIKPIFTDESYLELYRKQKKHLNTQQLTAFQLLFAWRDKTARREDESYGYVLPNHMMLKIAEELPKEPQGIIACCNPVPPLVRQQINEMHLLIQQAREMPLLKSEVAAGVKKSGPLPSAERLENVLFGPHDCSHAPPDGYPIIPTSGSVPVQKQASLFPDEKEDNLLGTTCLIATAVITLFNEPSAEDSKKGPLTVAQKKAQNIMESFENPFRMFLPSLGHRAPVSQAAKFDPSTKIYEISNRWKLAQVQVQKDSKEAVKKKAAEQTAAREQAKEACKAAAEQAISVRQQVVLENAAKKRERATSDPRTTEQKQEKKRLKISKKPKDPEPPEKEFTPYDYSQSDFKAFAGNSKSKVSSQFDPNKQTPSGKKCIAAKKIKQSVGNKSMSFPTGKSDRGFRYNWPQR

SEQ ID NO: 35 - DNA, 2759 bp, Homo sapiens
  CDS (51)..(2633)
gtctcctcggccgacaagctctcgcgagacgagccgtgcaggctgaaaaaatggcgccacccagtacccgggagcccagggtcctgtcggcgaccagcgcaaccaaatccgacggagagatggtgctgccaggcttcccggacgccgacagctttgtgaagtttgctcttgggtccgtggtggcagtcaccaaggcatctgggggcctaccacagtttggcgatgagtatgatttttaccgaagttttcctggcttccaagcattttgcgaaacacagggagacaggttgcttcagtgcatgagcagagtaatgcagtaccatgggtgtcgcagcaacattaaggatcgaagtaaagtgactgagctggaagacaagtttgatttactagttgatgccaatgatgtaattctggagagagtgggtattttactggatgaagcctcaggtgtaaacaagaatcaacagcctgtcctccctgccggcttgcaggtccccaaaacggtagtgtccagctggaaccgtaaggcagcagaatatggcaaaaaagcaaaatctgaaactttccggctgcttcatgcaaaaaatatcatccgacctcagctcaagtttcgagagaagattgacaattccaacacaccatttcttcctaaaatcttcatcaaacccaatgctcagaaacctctccctcaagctctctctaaggaaaggcgggaacgcccacaggatcgtcctgaggacttggacgtcccccctgcactggctgatttcatccatcagcagagaacccagcaggttgagcaagacatgtttgcacatccttatcaatatgaactaaatcactttaccccagcagatgcagtgcttcaaaagccacaaccccagttatacagacctatagaagagacaccatgccatttcatatcctccctggatgaactcgtggaactcaacgaaaagctcttgaattgtcaggaatttgcagttgacttggagcaccactcttacaggagcttcctgggactgacctgcctgatgcaaatttctactcggacggaagacttcatcattgacaccctcgagcttcgaagtgacatgtacattctcaatgagagcctcacagacccagccatcgttaaggtctttcatggtgctgattcagacatagaatggctacagaaagactttgggttgtatgtagtaaacatgtttgatactcatcaggcagcacgccttcttaacctgggcaggcactcactcgatcatctcctgaaactctactgcaacgtggactcaaacaagcaatatcagctggctgattggagaatacgccctctgcccgaggagatgctcagctacgcccgggatgacacccattacctgctatatatctatgacaaaatgaggctggagatgtgggagcgcggcaacgggcagccggtgcagctgcaggtggtgtggcaacggagcagggacatctgcctcaagaaattcatcaaacctatcttcacggatgagtcctaccttgaactctataggaagcagaagaagcaccttaacacacagcagttgacagcctttcagctgctgtttgcctggagggataaaacagctcgcagggaagatgaaagttacggatatgtactgccaaaccacatgatgctgaaaatagctgaagaactgcctaaggaacctcagggcatcatagcttgctgcaacccagtaccgccccttgtgcggcagcagatcaacgaaatgcaccttttaatccagcaggcccgagagatgcccctgctcaagtctgaagttgcagccggagtgaagaagagcggaccgctgcccagtgctgagagattggagaatgttctctttggacctcacgactgctcccatgcccctccggatggctatccaatcatcccaaccagtggatctgtgccagttcagaagcaggcgagcctcttccctgatgaaaaagaagataacttgctgggtaccacatgcctgattgccacagctgtcatcacgttatttaatgaacctagtgctgaagacagtaaaaagggtccattgacagttgcacagaaaaaagcccagaacatcatggagtcctttgaaaatccatttaggatgatcagcaaccgctggaagctggcccaggtacaagtacaaaaagactctaaagaagctgtcaagaagaaggcagctgagcaaacagctgcccgggaacaggcaaaggaggcgtgcaaagctgcagcagaacaggccatctccgtccgacagcaggtcgtgctagaaaatgctgcaaagaagagagagcgagcaacaagcgacccaaggaccacagaacagaaacaagagaagaaacgactcaaaatttccaagaagccaaaggacccagagccaccagaaaaagagtttacgccttacgactacagccagtcagacttcaaggcttttgctggaaacagcaaatccaaagtttcttctcagtttgatccaaataaacagaccccgtctggcaagaaatgcattgcagccaaaaaaattaaacagtcggtgggaaacaaaagcatgtcctttccaactggaaagtcagacagaggcttcaggtacaactggccacagagatagtcctggaagacacgtggcgcctgtggaccggaagcaccaaatgctggtgctgcttttgtacatacatatttttaaaccattaaaattcttcctgaagaaaaaaaaaaaaaaaaaaaaaaaaaaaaa

SEQ ID NO: 36 - protein, 860 aa, Homo sapiens
MAPPSTREPRVLSATSATKSDGEMVLPGFPDADSFVKFALGSVVAVTKASGGLPQFGDEYDFYRSFPGFQAFCETQGDRLLQCMSRVMQYHGCRSNIKDRSKVTELEDKFDLLVDANDVILERVGILLDEASGVNKNQQPVLPAGLQVPKTVVSSWNRKAAEYGKKAKSETFRLLHAKNIIRPQLKFREKIDNSNTPFLPKIFIKPNAQKPLPQALSKERRERPQDRPEDLDVPPALADFIHQQRTQQVEQDMFAHPYQYELNHFTPADAVLQKPQPQLYRPIEETPCHFISSLDELVELNEKLLNCQEFAVDLEHHSYRSFLGLTCLMQISTRTEDFIIDTLELRSDMYILNESLTDPAIVKVFHGADSDIEWLQKDFGLYVVNMFDTHQAARLLNLGRHSLDHLLKLYCNVDSNKQYQLADWRIRPLPEEMLSYARDDTHYLLYIYDKMRLEMWERGNGQPVQLQVVWQRSRDICLKKFIKPIFTDESYLELYRKQKKHLNTQQLTAFQLLFAWRDKTARREDESYGYVLPNHMMLKIAEELPKEPQGIIACCNPVPPLVRQQINEMHLLIQQAREMPLLKSEVAAGVKKSGPLPSAERLENVLFGPHDCSHAPPDGYPIIPTSGSVPVQKQASLFPDEKEDNLLGTTCLIATAVITLFNEPSAEDSKKGPLTVAQKKAQNIMESFENPFRMISNRWKLAQVQVQKDSKEAVKKKAAEQTAAREQAKEACKAAAEQAISVRQQVVLENAAKKRERATSDPRTTEQKQEKKRLKISKKPKDPEPPEKEFTPYDYSQSDFKAFAGNSKSKVSSQFDPNKQTPSGKKCIAAKKIKQSVGNKSMSFPTGKSDRGFRYNWPQR

SEQ ID NO: 37 - DNA, 3316 bp, Homo sapiens
  CDS (307)..(2772)
cactctctttctctctccctctggcatgcatgctgctggtaggagacccccaagtcaacattgcttcagaaatcctttagcactcatttctcaggagaacttatggcttcagaatcacagctcggtttttaagatggacataacctgtacgaccttctgatgggctttcaactttgaactggatgtggacacttttctctcagatgacagaattactccaacttcccctttgcagttgcttcctttccttgaaggtagctgtatcttattttctttaaaaagctttttcttccaaagccacttgccatgccgaccgtcattagcgcatctgtggctccaaggacagcggctgagccccggtccccagggccagttcctcacccggcccagagcaaggccactgaggctgggggtggaaacccaagtggcatctattcagccatcatcagccgcaattttcctattatcggagtgaaagagaagacattcgagcaacttcacaagaaatgtctagaaaagaaagttctttatgtggaccctgagttcccaccggatgagacctctctcttttatagccagaagttccccatccagttcgtctggaagagacctccggaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgaaaaagaaaaaaaccaagcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgctcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtc

SEQ ID NO: 38 - protein, 821 aa, Homo sapiens
MPTVISASVAPRTAAEPRSPGPVPHPAQSKATEAGGGNPSGIYSAIISRNFPIIGVKEKTFEQLHKKCLEKKVLYVDPEFPPDETSLFYSQKFPIQFVWKRPPEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVKKKKTKPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 39 - DNA, 3298 bp, Homo sapiens
  CDS (307)..(2754)
cactctctttctctctccctctggcatgcatgctgctggtaggagacccccaagtcaacattgcttcagaaatcctttagcactcatttctcaggagaacttatggcttcagaatcacagctcggtttttaagatggacataacctgtacgaccttctgatgggctttcaactttgaactggatgtggacacttttctctcagatgacagaattactccaacttcccctttgcagttgcttcctttccttgaaggtagctgtatcttattttctttaaaaagctttttcttccaaagccacttgccatgccgaccgtcattagcgcatctgtggctccaaggacagcggctgagccccggtccccagggccagttcctcacccggcccagagcaaggccactgaggctgggggtggaaacccaagtggcatctattcagccatcatcagccgcaattttcctattatcggagtgaaagagaagacattcgagcaacttcacaagaaatgtctagaaaagaaagttctttatgtggaccctgagttcccaccggatgagacctctctcttttatagccagaagttccccatccagttcgtctggaagagacctccggaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgctcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtc

SEQ ID NO: 40 - protein, 815 aa, Homo sapiens
MPTVISASVAPRTAAEPRSPGPVPHPAQSKATEAGGGNPSGIYSAIISRNFPIIGVKEKTFEQLHKKCLEKKVLYVDPEFPPDETSLFYSQKFPIQFVWKRPPEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 41 - DNA, 3040 bp, Homo sapiens
  CDS (307)..(2496)
cactctctttctctctccctctggcatgcatgctgctggtaggagacccccaagtcaacattgcttcagaaatcctttagcactcatttctcaggagaacttatggcttcagaatcacagctcggtttttaagatggacataacctgtacgaccttctgatgggctttcaactttgaactggatgtggacacttttctctcagatgacagaattactccaacttcccctttgcagttgcttcctttccttgaaggtagctgtatcttattttctttaaaaagctttttcttccaaagccacttgccatgccgaccgtcattagcgcatctgtggctccaaggacagcggctgagccccggtccccagggccagttcctcacccggcccagagcaaggccactgaggctgggggtggaaacccaagtggcatctattcagccatcatcagccgcaattttcctattatcggagtgaaagagaagacattcgagcaacttcacaagaaatgtctagaaaagaaagttctttatgtggaccctgagttcccaccggatgagacctctctcttttatagccagaagttccccatccagttcgtctggaagagacctccggaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgctcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtc

SEQ ID NO: 42 - protein, 729 aa, Homo sapiens
MPTVISASVAPRTAAEPRSPGPVPHPAQSKATEAGGGNPSGIYSAIISRNFPIIGVKEKTFEQLHKKCLEKKVLYVDPEFPPDETSLFYSQKFPIQFVWKRPPEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 43 - DNA, 1661 bp, Homo sapiens
  CDS (170)..(1099)
ccctcctccacgcttacagccacacacacagtcacacagacgcgttctgagggtggctgcccgcttgggatggaggaatcacttccctcagaacccagccaagtcctctaggcctccttgggggtccttccagcctgaggggcttcggagctgaggagcagctgttctgatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgaaaaagaaaaaaaccaagcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgctcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtcaaaaaaaaaaaaaaaaaa

SEQ ID NO: 44 - protein, 309 aa, Homo sapiens
MHGNKQHLQKDFFLYNASKARSKTYINMREVSQRFRLPPSEYVIVPSTYEPHQEGEFILRVFSEKRNLSEEVENTISVDRPVKKKKTKPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 45 - DNA, 1291 bp, Homo sapiens
  CDS (259)..(729)
cataatcctgaccctgagccagtgccaggtctccaagtgccttctgaatgaccacaggcgattggttttagtggtaggtgtgtggggatctgttctggtcatctggatgctggtcatcggtgtgcagtattgatcaggacctgcaaacccaaaagcttatgggagctggcacgtcacccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgctcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtcaaaaaaaaaaaaaaaaaa

SEQ ID NO: 46 - protein, 156 aa, Homo sapiens
MEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 47 - DNA, 1309 bp, Homo sapiens
  CDS (277)..(747)
cataatcctgaccctgagccagtgccaggtctccaagtgccttctgaatgaccacaggcgattggttttagtggtaggtgtgtggggatctgttctggtcatctggatgctggtcatcggtgtgcagtattgatcaggacctgcaaacccaaaagcttatgggagctggcacgtcacaaaaagaaaaaaaccaagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgctcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtcaaaaaaaaaaaaaaaaaa

SEQ ID NO: 48 - protein, 156 aa, Homo sapiens; identical to SEQ ID NO: 46

SEQ ID NO: 49 - DNA, 3243 bp, Homo sapiens
  CDS (495)..(2681)
ctattccagtgtttcagcgaggtggaagtgtgataccaataaagacaactgtaggaaaatccacaggctggatgactgaatcctcctatggactccgggttgctctaagcactaagggttcttcagtgggtgagttatatcttgatgatggccattcattccaatacctccaccagaagcaatttttgcacaggaagttttcattctgttccagtgttctgatcaatagttttgctgaccagaggggtcattatcccagcaagtgtgtggtggagaagatcttggtcttaggcttcaggaaggagccatcttctgtgactacccactcatctgatggtaaagatcagcctgtggcttttacgtattgtgccaaaacatccatcctgagcctggagaagctctcactcaacattgccactgactgggagggttcctaaaaaagctctaaaatgaatgcatagtgtgtctttccttcaagatgtaaatgtcaaggaatgagttggcaaatcagtttaaaaacacaaactacccaacagaaaaatgaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgttcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtcaaaaaaaaaaaaaaaaaa

SEQ ID NO: 50 - protein, 728 aa, Homo sapiens
MSWQISLKTQTTQQKNEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 51 - DNA, 3901 bp, Homo sapiens
  CDS (495)..(1775)
ctattccagtgtttcagcgaggtggaagtgtgataccaataaagacaactgtaggaaaatccacaggctggatgactgaatcctcctatggactccgggttgctctaagcactaagggttcttcagtgggtgagttatatcttgatgatggccattcattccaatacctccaccagaagcaatttttgcacaggaagttttcattctgttccagtgttctgatcaatagttttgctgaccagaggggtcattatcccagcaagtgtgtggtggagaagatcttggtcttaggcttcaggaaggagccatcttctgtgactacccactcatctgatggtaaagatcagcctgtggcttttacgtattgtgccaaaacatccatcctgagcctggagaagctctcactcaacattgccactgactgggagggttcctaaaaaagctctaaaatgaatgcatagtgtgtctttccttcaagatgtaaatgtcaaggaatgagttggcaaatcagtttaaaaacacaaactacccaacagaaaaatgaaatttgcgagaatccccgatttatcattgatggagccaacagaactgacatctgtcaaggagagctaggggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagaggtatagcagcagcagcggccagcagttgtgtgcagcactacccagggggccccgagtctgtctgtggctcgtcgagaagcttcctggtggggtttgtgggcaggactgtgataggagagggccttgcctgtgttatttccactgcagagcaggtgcctcagggcattgcatgacccatgactaccacccccaggatgtgcactttctccctcgcaccagacactgcacgtcacacacatgcctttgcacactcaccctcctccacgcttacagccacacacacagtcacacagacgcgttctgagggtggctgcccgcttgggatggaggaatcacttccctcagaacccagccaagtcctctaggcctccttgggggtccttccagcctgaggggcttcggagctgaggagcagctgttctggtaagtgtccctgagtgtggggatgacacatttgccattcactctgaatcacaacagaaaaggaagaggaagtgaggtagggaggctatttaagccttgggagtcggaagtagggaggttgaaactgtgacatgggtgaccagggagttgggaagggacccttggaggtggctgtggcaggacaggacgttcctcccgaggggctcatgtgccctgggctctccccatctctcagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgttcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtcaaaaaaaaaaaaaaaaaa

SEQ ID NO: 52 - protein, 426 aa, Homo sapiens
MSWQISLKTQTTQQKNEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEV

SEQ ID NO: 53 - DNA, 3521 bp, Homo sapiens
  CDS (495)..(818)
ctattccagtgtttcagcgaggtggaagtgtgataccaataaagacaactgtaggaaaatccacaggctggatgactgaatcctcctatggactccgggttgctctaagcactaagggttcttcagtgggtgagttatatcttgatgatggccattcattccaatacctccaccagaagcaatttttgcacaggaagttttcattctgttccagtgttctgatcaatagttttgctgaccagaggggtcattatcccagcaagtgtgtggtggagaagatcttggtcttaggcttcaggaaggagccatcttctgtgactacccactcatctgatggtaaagatcagcctgtggcttttacgtattgtgccaaaacatccatcctgagcctggagaagctctcactcaacattgccactgactgggagggttcctaaaaaagctctaaaatgaatgcatagtgtgtctttccttcaagatgtaaatgtcaaggaatgagttggcaaatcagtttaaaaacacaaactacccaacagaaaaatgaaatttgcgagaatccccgatttatcattgatggagccaacagaactgacatctgtcaaggagagctagacgggttccagggcagctgcctacctggccttccttccaatacaaatcatcttggtggatggttctctgaggctcagtcttcgctgaagtcagaagaggaattggactcacattgcaaaggcacagggcagggcagatttcctacaggtgttaggaagaacaacccagttatgatcacctactgctctgtctccattgaggcctaaaaaggaagtgagtttatactgcagttggaggaactgcctgcagccttgaggaaaatgtctagtcacaagggagggactgctggtttctcgcagccattgcctgcctgaccctgaaccagcaccttcttttccgagtcataccccatgatcaaagtttcatcgaaaactacgcagggatcttccacttccagttctggcgctatggagagtgggtggacgtggttatagatgactgcctgccaacgtacaacaatcaactggttttcaccaagtccaaccaccgcaatgagttctggagtgctctgctggagaaggcttatgctaagctccatggttcctacgaagctctgaaaggtgggaacaccacagaggccatggaggacttcacaggaggggtggcagagttttttgagatcagggatgctcctagtgacatgtacaagatcatgaagaaagccatcgagagaggctccctcatgggctgctccattgatgatggcacgaacatgacctatggaacctctccttctggtctgaacatgggggagttgattgcacggatggtaaggaatatggataactcactgctccaggactcagacctcgaccccagaggctcagatgaaagaccgacccggacaatcattccggttcagtatgagacaagaatggcctgcgggctggtcagaggtcacgcctactctgtcacggggctggatgaggtcccgttcaaaggtgagaaagtgaagctggtgcggctgcggaatccgtggggccaggtggagtggaacggttcttggagtgatagatggaaggactggagctttgtggacaaagatgagaaggcccgtctgcagcaccaggtcactgaggatggagagttctggatgtcctatgaggatttcatctaccatttcacaaagttggagatctgcaacctcacggccgatgctctgcagtctgacaagcttcagacctggacagtgtctgtgaacgagggccgctgggtacggggttgctctgccggaggctgccgcaacttcccagatactttctggaccaaccctcagtaccgtctgaagctcctggaggaggacgatgaccctgatgactcggaggtgatttgcagcttcctggtggccctgatgcagaagaaccggcggaaggaccggaagctaggggccagtctcttcaccattggcttcgccatctacgaggttcccaaagagatgcacgggaacaagcagcacctgcagaaggacttcttcctgtacaacgcctccaaggccaggagcaaaacctacatcaacatgcgggaggtgtcccagcgcttccgcctgcctcccagcgagtacgtcatcgtgccctccacctacgagccccaccaggagggggaattcatcctccgggtcttctctgaaaagaggaacctctctgaggaagttgaaaataccatctccgtggatcggccagtgcccatcatcttcgtttcggacagagcaaacagcaacaaggagctgggtgtggaccaggagtcagaggagggcaaaggcaaaacaagccctgataagcaaaagcagtccccacagccacagcctggcagctctgatcaggaaagtgaggaacagcaacaattccggaacattttcaagcagatagcaggagatgacatggagatctgtgcagatgagctcaagaaggtccttaacacagtcgtgaacaaacacaaggacctgaagacacacgggttcacactggagtcctgccgtagcatgattgcgctcatggatacagatggctctggaaagctcaacctgcaggagttccaccacctctggaacaagattaaggcctggcagaaaattttcaaacactatgacacagaccagtccggcaccatcaacagctacgagatgcgaaatgcagtcaacgacgcaggattccacctcaacaaccagctctatgacatcattaccatgcggtacgcagacaaacacatgaacatcgactttgacagtttcatctgctgcttcgttaggctggagggcatgttcagagcttttcatgcatttgacaaggatggagatggtatcatcaagctcaacgttctggagtggctgcagctcaccatgtatgcctgaaccaggctggcctcatccaaagccatgcaggatcactcaggatttcagtttcaccctctatttccaaagccatttacctcaaaggacccagcagctacacccctacaggcttccaggcacctcatcagtcatgttcctcctccattttaccccctacccatccttgatcggtcatgcctagcctgaccctttagtaaagcaatgaggtaggaagaacaaacccttgtccctttgccatgtggaggaaagtgcctgcctctggtccgagccgcctcggttctgaagcgagtgctcctgcttaccttgctctaggctgtctgcagaagcacctgccggtggcactcagcacctccttgtgctagagccctccatcaccttcacgctgtcccaccatgggccaggaaccaaaccagcactgggttctactgctgtggggtaaactaactcagtggaatagggctggttactttgggctgtccaactcataagtttggctgcattttgaaaaaagctgatctaaataaaggcatgtgtatggctggtcaaaaaaaaaaaaaaaaaa

SEQ ID NO: 54 - protein, 107 aa, Homo sapiens
MSWQISLKTQTTQQKNEICENPRFIIDGANRTDICQGELDGFQGSCLPGLPSNTNHLGGWFSEAQSSLKSEEELDSHCKGTGQGRFPTGVRKNNPVMITYCSVSIEA

SEQ ID NO: 55 - DNA, 2665 bp, Homo sapiens
  CDS (42)..(872)
ggctctgggcatcaccagcggccccagggaaaaagaaagaaatgggaaacagcatgaaatccacccctgcgcctgccgagaggcccctgcccaacccggagggactggatagcgacttccttgccgtgctaagtgactacccgtctcctgacatcagccccccgatattccgccgaggggagaaactgcgtgtgatttctgatgaagggggctggtggaaagctatttctcttagcactggtcgagagagttacatccctggaatatgtgtggccagagtttaccatggctggctgtttgagggcctgggcagagacaaggccgaggagctgctgcagctgccagacacaaaggtcggctccttcatgatcagagagagtgagaccaagaaagggttttactcactgtcggtgagacacaggcaggtaaagcattaccgcattttccgtctgccgaacaactggtactacatttccccgaggctcaccttccagtgcctggaggacctggtgaaccactattctgaggtggctgatggcctgtgctgtgtgctcaccacgccctgcctgacacaaagcacggctgccccagcagtgagggcctccagctcacctgtcaccttgcgtcagaagactgtggactggaggagagtgtccagactgcaggaggaccccgagggaacagagaacccgcttggggtagacgagtcccttttcagctatggccttcgagagagcattgcctcttacctgtccctgaccagtgaggacaacacctcctttgatcgaaagaagaaaagcatctccctgatgtatggtggcagcaagagaaagagctcattcttctcatcaccaccttactttgaggactagccaagaacagacacaatggttcatgcccaaaaggaacagaagttccaactattgcctgggatcttgcgaaaagcgaggttccctgatccctgggagcctcacgtattttagaagccaagagaagccacatggagactcaaattcgcatcttctctatccacatcatgaccaaaggaacccctccctggtgtctgatcagggctgtggcatcacaaaacattggatcatgacatgtcgggcgatgcttggaaaagcccagcatgtatgtatgcacacattgtgtgtgtgggaaggacaaagccactctcacaagaaagggcaccaggactgctctccaaggaactggacctgtccagacagttacactccaaggtcattggagagaacttctgtatgggcaagcctgagagggagaggaaacaaaagctgtgtcctggcagaaggtctgggtttgcagatgggtgccctgaatggaactactttaactaatccatagggacttctggtatgctttcctctctttttaaaggaacttcgtgacactaaacattagcccaaaggacttcttagccttcaattgggagatacctttggtctgctcctgcaccaaagccatatgggtggaagtcagttggcctccctggttctgcagagggccagaagaatgagagagaggaagactgctggcagggaaatcgaggaggcgagactagaactgcaccagcttccctgatgtctgcagccatggctttgcagcgcaaacagaacttctctgggatgctgggattcttgcctgtatgaatgcatcaagtattcatttattgcccgaataggcattgcattaagtcctctgttaggtgtcaggcaagccaaaaaaaaaaaaaagatgctaagtcctaacccccaacagaagtgttcacagtgtagacgggaaaaaatgtataaacaaatgtgtaaaaagagaaatcagctcatggcttaggatggaattagagacaggtgagggacactcaggagctcattttccagctgctcttcagagtggaagggctggctggatcgggtaggtaagaatagctggattttttagaaaagaaatggatacagtctaaagaattaactcacccggtactttattctaagaagggtctggcatccatatgaggaaaaatgctcagctccaggaaagatggggagtccaagtggattaatgatgtcatgcataattttaagagacaagggagaaaacacaatgtatagccagagaaggagaagctcccatccaaatcctactaggaagagagtgggctgcagatgaatctgtgactcatgtttccctgtttcaaagggatcctggggaaggaggggaacatgcttgcagtatctctccctgtctgtctgctcacataagcattccgtccatctaagctcatcgtgctactggtatgtgtatgtgcagttacacagtttcctgtatcataaatcctagtgtgtttatacaaggagacatctgtggtttccccaaccgttccaaaaggctatttcaaaggaaccagcccacgtatgagaaatgaatgtaacactgtggacattgacttcccgcataaggcagggtgaccccctgaactccagatgtttgcacagtatcttatgtgttgttttccgttgtgacgaatgtgattggaacatttggggagcacccagagggatttttcagtgggaagcattacactttgctaaatcatgtatttattcctgattaaaacaaacctaataaatatttaacccttggc

SEQ ID NO: 56 - protein, 276 aa, Homo sapiens
MGNSMKSTPAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSFDRKKKSISLMYGGSKRKSSFFSSPPYFED

SEQ ID NO: 57 - DNA, 1887 bp, Homo sapiens
  CDS (137)..(1591)
gtgttttcgagacgagtctcgacgcagcagctgtcagctccattttgttgttggtgcgcgacgcagtcagctgcgtgattcccgtgattgcgttacaagctttgtctccttcgacttggagtctttgtccaggacgatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatagatctgtaattggacagctctctcgaagagatcttacagactgtatcagtctaatttttaaattttaagttattttgtacagctttgtaaattcttaacatttttatattgccatgtttattttgtttgggtaatttggttcattaagtacatagctaaggtaatgaacatctttttcagtaattgtaaagtgatttattcagaataaattttttgtgcttatgaagttgatatgtatctgaacagtttgttctaagtaccatttttcttcctacttctattaaagaatggacatag

SEQ ID NO: 58 - protein, 484 aa, Homo sapiens
MRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRRCKPLKEFMLSQDVEHERLFDLIQKMLEYDPAKRITLREALKHPFFDLLKKSI

SEQ ID NO: 59 - DNA, 1796 bp, Homo sapiens
  CDS (137)..(547)
gtgttttcgagacgagtctcgacgcagcagctgtcagctccattttgttgttggtgcgcgacgcagtcagctgcgtgattcccgtgattgcgttacaagctttgtctccttcgacttggagtctttgtccaggacgatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatagatctgtaattggacagctctctcgaagagatcttacagactgtatcagtctaatttttaaattttaagttattttgtacagctttgtaaattcttaacatttttatattgccatgtttattttgtttgggtaatttggttcattaagtacatagctaaggtaatgaacatctttttcagtaattgtaaagtgatttattcagaataaattttttgtgcttatgaagttgatatgtatctgaacagtttgttctaagtaccatttttcttcctacttctattaaagaatggacatag

SEQ ID NO: 60 - protein, 136 aa, Homo sapiens
MRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGMKLLIL

SEQ ID NO: 61 - DNA, 1000 bp, Homo sapiens
  CDS (346)..(762)
aacctcacgaggccttacgtgcctagagcggttcctctccgtggcgccctaccgccttcggcgcgaaggtggctggtgcgtaccgtcccgaaccctaggagccgaggtgagcgccgcgaagacccccgacattaagaagtggtgtccaccgcagcccccttaagccggataccctcccccaccccgaggagcaagcctgccttccgccacgcgccgcccgtcttccgcgcccgcgcagaacctcgcggctgtgcccgcctaaagccacgcccatcctctcttggccacgccctcagagactcctcccctgtcgcccagatcccacccgcaggaccccaaaacccaatgatcctgcagcagcccttgcagcgaggcccccagggaggggcccagcgcctcccgcgggccgccttgggggtgacttggggcctggacgccagctcccctctccgaggagctgtgcccatgagcaccaagcggcgcctggaggaggagcaggagcctctgcgcaagcagtttctgtctgaggagaacatggccacccacttctctcaactcagcctgcacaatgaccacccctactgcagcccccccatgaccttctccccagccctgcccccactcaggagcccttgctctgagctgcttctctggcgctatcctggcagcctcatccctgaggccctccgtctgctgaggctgggggacacccccagtcccccctaccctgcaaccccagctggggacataatggagctctgagtgctggtggacagtgcccctcccaccttccttcttccccacaacagaagagaccagcgactcccgcaaagggacaaggttcctccctctcctgcagagtaggcatctgggcaccaagaccttccctcaacagaggacactgagcccaacggagttctgggatgggaggggtgggagcatgggaagggaggcatcccacccccaagaagaactgaataaagattgctgagcaaaggaa

SEQ ID NO: 62 - protein, 138 aa, Homo sapiens
MILQQPLQRGPQGGAQRLPRAALGVTWGLDASSPLRGAVPMSTKRRLEEEQEPLRKQFLSEENMATHFSQLSLHNDHPYCSPPMTFSPALPPLRSPCSELLLWRYPGSLIPEALRLLRLGDTPSPPYPATPAGDIMEL

SEQ ID NO: 63 - DNA, 943 bp, Homo sapiens
  CDS (346)..(705)
aacctcacgaggccttacgtgcctagagcggttcctctccgtggcgccctaccgccttcggcgcgaaggtggctggtgcgtaccgtcccgaaccctaggagccgaggtgagcgccgcgaagacccccgacattaagaagtggtgtccaccgcagcccccttaagccggataccctcccccaccccgaggagcaagcctgccttccgccacgcgccgcccgtcttccgcgcccgcgcagaacctcgcggctgtgcccgcctaaagccacgcccatcctctcttggccacgccctcagagactcctcccctgtcgcccagatcccacccgcaggaccccaaaacccaatgatcctgcagcagcccttgcagcgaggcccccagggaggggcccagcgcctcccgcgggccgccttgggggtgacttggggcctggacgccagggagcctctgcgcaagcagtttctgtctgaggagaacatggccacccacttctctcaactcagcctgcacaatgaccacccctactgcagcccccccatgaccttctccccagccctgcccccactcaggagcccttgctctgagctgcttctctggcgctatcctggcagcctcatccctgaggccctccgtctgctgaggctgggggacacccccagtcccccctaccctgcaaccccagctggggacataatggagctctgagtgctggtggacagtgcccctcccaccttccttcttccccacaacagaagagaccagcgactcccgcaaagggacaaggttcctccctctcctgcagagtaggcatctgggcaccaagaccttccctcaacagaggacactgagcccaacggagttctgggatgggaggggtgggagcatgggaagggaggcatcccacccccaagaagaactgaataaagattgctgagcaaaggaa

SEQ ID NO: 64 - protein, 119 aa, Homo sapiens
MILQQPLQRGPQGGAQRLPRAALGVTWGLDAREPLRKQFLSEENMATHFSQLSLHNDHPYCSPPMTFSPALPPLRSPCSELLLWRYPGSLIPEALRLLRLGDTPSPPYPATPAGDIMEL

SEQ ID NO: 65 - DNA, 788 bp, Homo sapiens
  CDS (134)..(550)
aacctcacgaggccttacgtgcctagagcggttcctctccgtggcgccctaccgccttcggcgcgaaggtggctggtgcgtaccgtcccgaaccctaggagccgagatcccacccgcaggaccccaaaacccaatgatcctgcagcagcccttgcagcgaggcccccagggaggggcccagcgcctcccgcgggccgccttgggggtgacttggggcctggacgccagctcccctctccgaggagctgtgcccatgagcaccaagcggcgcctggaggaggagcaggagcctctgcgcaagcagtttctgtctgaggagaacatggccacccacttctctcaactcagcctgcacaatgaccacccctactgcagcccccccatgaccttctccccagccctgcccccactcaggagcccttgctctgagctgcttctctggcgctatcctggcagcctcatccctgaggccctccgtctgctgaggctgggggacacccccagtcccccctaccctgcaaccccagctggggacataatggagctctgagtgctggtggacagtgcccctcccaccttccttcttccccacaacagaagagaccagcgactcccgcaaagggacaaggttcctccctctcctgcagagtaggcatctgggcaccaagaccttccctcaacagaggacactgagcccaacggagttctgggatgggaggggtgggagcatgggaagggaggcatcccacccccaagaagaactgaataaagattgctgagcaaaggaa

SEQ ID NO: 66 - protein, 138 aa, Homo sapiens; identical to SEQ ID NO: 62

SEQ ID NO: 67 - DNA, 1904 bp, Homo sapiens
  CDS (239)..(898)
gcccgcgcgggatccgggtggctgcaggctgctggcttctgcggctgcggggtcggggtcgcggccagggccaagccgcagcgagttcacaggcggaacccctgcaggcggcgccccctacgcgaggtcacccctgggaaggagcgcagcccacccggcccctccgcatccgagcaggacgcccgtctcctctccctgaggatttcaggtctccctgtcccaggaggcttgtgccaagatggcatcagctggagacacccaggcaggcccacgggatgcagcagatcagaacttcgactatatgttcaaactgctactgataggcaacagcagtgtgggcaagacttccttcctgttccgatacgcggacgactccttcactcccgccttcgtcagtactgtgggcatcgatttcaaggtcaagaccgtctaccgccatgacaagaggatcaagctgcagatctgggacacagcgggccaggagcgctaccgcaccatcaccacggcctactaccggggagccatgggcttcctgctcatgtatgacatcgccaatcaggaatcctttgccgctgtgcaggactgggccacgcaaatcaagacctactcctgggacaacgcccaggtcatcctggtggggaacaagtgtgacctggaggacgaacgtgttgtgcctgctgaggatggccggaggctcgccgacgaccttggtttcgagttctttgaagccagtgccaaggagaacatcaatgtgaagcaggtcttcgagcgcctggtggatgtcatctgcgagaagatgaacgagtccctggaacccagctccagctcaggcagcaacgggaaaggcccggccgtgggggatgctccagccccccagcccagcagctgcagctgctagagatggcccccaaccccccacccacccctctcctgcctcggcagggactgtgatcgaggcgtgagccacagtggttatctccaagctcagggcaatcccttccctcctgtcaactgctccccgactcctgcatggctcattccttcacaatgtcgccgtggctgccaggttcctcaggcagccccggtgggaggctgcggaccatggggactcagctctgcatggcaggcagaggcccagggacactgacgtgcatgagcttgctgctttccagggtgtgttgagggtggccatgggtgacacgtcccactctttctgatggaggggcatccgtggtgccttctagcttagaaccattttccaggtctcttctgggttgggcaggactgatattcccaagctggtggaagatggttcatcttcctcgcacagtgtgggttcttggagttcatccagggaaggcggcgcctctttctcaggtcctgcaggctggtctctgagcctgcccccacgaactttctggattccaaggtgggatggtgatccctttgacccctgcagaccctctacttgcgaaaagcagcattgaagcagccttttcccattgtagaagggacagggagtcagatcccctcaaccccccggcttcagggaccccagaagtgccttccaagcttcccccaagatccacatcacccacgaccctgccactgtttttgctgtgttcaccacatctctgatcctgaagtgaattctggaactatggaccacattagactgggcagagattcgcccaacccctagcactccggctctgggctttcggccagaagttaattaacctcagagccaggagcctggggctggggcctccctcccaggccctgtttagctgtttagctggttgtgttttaagcaagttcttgcccaatcatgcatcctgctagaaaaggaaatgaagcaacgttcccagccaccgcaaaaaaaaaaaaaaaaaaaaaaaaaa

SEQ ID NO: 68 - protein, 219 aa, Homo sapiens
MASAGDTQAGPRDAADQNFDYMFKLLLIGNSSVGKTSFLFRYADDSFTPAFVSTVGIDFKVKTVYRHDKRIKLQIWDTAGQERYRTITTAYYRGAMGFLLMYDIANQESFAAVQDWATQIKTYSWDNAQVILVGNKCDLEDERVVPAEDGRRLADDLGFEFFEASAKENINVKQVFERLVDVICEKMNESLEPSSSSGSNGKGPAVGDAPAPQPSSCSC

SEQ ID NO: 69 - DNA, 567 bp, Homo sapiens
  CDS (99)..(476)
cagcgggcacgtgacatggccccggggagccgaggtgagcgtccagcttccggagccggagggggcccggcgtacccagcccccagcccgacgtgaccatgctgtcccgcctcctaaaagaacaccaggccaagcagaatgaacgcaaggagctgcaggaaaagaggaggcgagaggctatcactgcagcgacctgcctgacagaagctttggtggatcacctcaatgtgggtgtggcccaggcctacatgaaccagagaaagctggaccatgaggtgaagaccctacaggtccaggctgcccaatttgccaagcagacaggccagtggatcggaatggtggagaacttcaaccaggcactcaaggaaattggggatgtggagaactgggctcggagcatcgagctggacatgcgcaccattgccactgcactggaatatgtctacaaagggcagctgcagtctgccccttcctagcccctgttccctccccaaaccctatccctcctacctcacccgcagggggaaggagggaggctgacaagccttgaataaaacacaagcctcc

SEQ ID NO: 70 - protein, 125 aa, Homo sapiens
MLSRLLKEHQAKQNERKELQEKRRREAITAATCLTEALVDHLNVGVAQAYMNQRKLDHEVKTLQVQAAQFAKQTGQWIGMVENFNQALKEIGDVENWARSIELDMRTIATALEYVYKGQLQSAPS

SEQ ID NO: 71 - DNA, 4868 bp, Homo sapiens
  CDS (148)..(1761)
agccctaggttctcctgaatccctgcggtcccagcgtcgctccggacgctgccaacctgttctccaccgtcgctcgacttccacctctaagactcccacgaaactcaggttgaataattcatcaaattacacaactgaactcaagacatggctgcccagtgtgtcacaaaggtggcgctgaatgtttcctgtgccaatcttttggataaagatatagggtcaaagtcagaccctttatgtgtgttgtttttgaatacaagtggtcaacagtggtatgaggttgagcgcacagaaaggattaagaattgcttgaatccccaattttccaagacatttattattgattactactttgaagtggttcagaaattgaaatttggggtttatgacatcgacaacaaaactattgagctgagtgatgatgacttcttaggggaatgtgaatgtacccttggacaaattgtttccagcaagaagctaactcgaccactggtgatgaaaactggcagacctgcaggaaaagggagcattacgatttcagctgaagaaataaaagataatagagtggtcttgtttgaaatggaagccagaaaactggataataaggatctatttggaaagtcagacccatacctggaattccacaagcagacatctgatggaaactggctaatggttcatcggacagaggttgttaaaaacaacttgaatcctgtttggaggcctttcaagatctctcttaactcactgtgttacggagatatggacaaaaccattaaggtggagtgttatgattatgacaatgatgggtcacatgatctcattggaacatttcagaccaccatgacaaaactgaaagaagcctccagaagctcacctgttgaatttgaatgcataaatgagaaaaaaaggcaaaagaaaaaaagctacaagaattcaggtgttatcagtgtgaaacagtgtgagattacagtagaatgcacattccttgactatataatgggaggatgtcagctgaattttactgtgggagtggacttcactggctccaatggtgacccaaggtctccagactcccttcattacatcagccccaatggcgttaatgagtatttgactgctctctggtctgtgggactggtcattcaagattatgatgctgataagatgtttccagcttttggttttggcgctcagatacctcctcagtggcaggtatcacatgaatttccaatgaacttcaacccatccaatccctactgcaatggaatccaaggcattgtagaggcgtatcggtcttgtcttcctcagataaaactctatggaccaactaatttttctccaatcataaatcacgtggccaggtttgctgctgcagccacgcaacagcagacagcttctcaatattttgtgcttttgattattactgatggtgtgatcacagaccttgatgaaaccagacaagctatagttaatgcctccaggctgcctatgtccatcataattgttggagttggaggtgctgacttcagcgccatggagtttctggatggtgatggtggaagtctccgctccccattgggcgaagtggccatcagagatattgtccagtttgtgcctttcagacagttccagaatgctccaaaagaagcacttgctcagtgtgtcttggcagagattccccagcaggtggtgggctacttcaatacatacaaactccttcctcccaagaacccagccacgaaacaacagaagcagtgaccacttcaacagaattcttttgtgttatgtggagcaatgccatctctcaccccaaatcgtgtatctgtcattctacgtactttttacccccagcatttatgatgtaaatctctttctctatggattatatctgtttaaagcattctttctaggttattttggggggacagtgccaagtccatctttgcccagtcaattcagtgattgatagcaatttacattaattgcagtaaagctctttggattagaaattagtgtggggaaagcttattctgttgttgtttttgtttactttcatatgatgaaaatgctgtgtttaagtgtttgtcaataggaagaatggaaaactgttgggatgatgtggtttgcaggttgctgtgcctgattcacagtgtatgttgtataagccaatgtccatacctgattatgagagcttcttaaattatatgatatcaaatttgttcctgtaactctgtatacagtgcttttctgcaaggtaaaaataacctgtctatgcatctgatttttgctacagtttagacactgtggtttacaaaacagcatgcactcaacttgggactttatgaaaagtactgaatgagcaggaaaaggcacatactcagttttttaaatgtacaatcaacaagtaaaaataacctcatgtaagtaagccatttttatttgcctttctagatattttattttattgtggaaaactgtaaacatggtcagatttggctttttttttcattaactgagcaagactttcaggatattgtagatgcacagatggtaggttgtcctgaattctacattattagattactttaattgagatttgttaaaacggttaggactgttttgtccaggaaagataagaggaccaaacatataaggtgaaattcagaattccgtttccttctaactaatgaaaaactgcttactaaaaaaaaattttatactttccttgctaaggtcccatatattgatttgtacagatccacttagtcattttctcctttttttaagaaccattttcatctgatttttaaactcacgataccagttatctgttaatcaaaattgcattttacaatttaataatgtgatatttcctatgtctacagcataccttattaggtataaaacctactgcaacttagaaaaaggaaagaaaaaagaaaacttttccaactgctgcattaagatagggtggattttatgtgctttttttttttaagagttgaatttcttttcctgacttttaccttttacagcgtattacttagtgaacattacattttcagaatagatcctaatattttattgagggcctatgtgctaaaaactatgcatatctatatattggccaattatctttaataatttaccttttgaaattgcatgtttatcatatatccttaagtggacacatacagtgccatgttgatgtgcctctcagttttattgaaaagctgccccacagcccatgtctcttgttctctgcaatgcctcaagggagtgagctctcaaccacagatagctgtggcttctcagaagcagctcattgccaaggccaggctgagaggggacctgcttgctgtggtggttgcctagcccagatgagcatttacctaccaccttcccacttggctagctgtcctttggatatgtgctgttaactggggaaggcatctaactagtagcctgctactccatagtatggctcaatagatgacacatcattttgacattatcaataggagaaaagaaaactaacccttcttctgattgtttggagccatagttgtctcagatgttctaattctctttgtatgcttggaaacagcatagatatgttgctgtggttttcagaattttctcttttaatcacaagaagccttttaaaaaatgacttacacatattctcaatgtacagtaaaacagacagaagtgagcttatctgtttgatgctgtggcagggtcccagtcactgggcatatcctccttctccttaaccagctccacagcagcccctgagtcacctgcacaaggtgcttgggaactgctggttatgagcattcctggttttcttcagccaaataacaggtaatcactgtcaattggatttggtcttcattattttatattctgattttatcagaattattctattttaaaattgttttaaaatttaaaacatttaattcatgatcatgttcatcagtagatgctattattcataagaactgtgattccagcaaactagggtaattggtgcctttttacagttttgaataaaagcatttacaatttctaaattatcagttttcacagtttcagcactcaacctcatcatacgctgatttaatattgttttacattaaaatagtccttttccctgttgtgccaccattcatttaagtgctgtttgttcttaaaatgcatttaaagaaaaattacccatattgactttcacacctcatataatcagatctattacaaatatatatcggagtgacggtgcccaggatagatgtaatatttcttacagatgctggcacagaggaaataatataccagctaatctagtcacctaaccttgtggttagaattgcaattttaagaccagaaaaatttgaagtctgatcagagatttacaactgttcattatagtggtgccttaggcaatctttccaaagtaaattcagggccccattgctacttatgccatatttggacatactttttttttcttcaattttgtaaacttcctggaaagctgtcttcactaagtatcccctagtctctatatatgtggttagtagtcatggaaatgacacataaagtacgccagaagtttgatggaacgtgttagaaactgttttgtgcttttatggatgtcatacttgacaatacatgtgtaagttactaatatatgaattgatgctaaatatatcttacatttgaattccttttggataaagttatttcttgatgtgacagagtagtgtgttttcatttttattctttacatgtgaccaaaacaatagaaaagttaaaaataaaatatagtgttttaggtggcaaaaaaaaaaaaaaaaaaa

SEQ ID NO: 72 - protein, 537 aa, Homo sapiens
MAAQCVTKVALNVSCANLLDKDIGSKSDPLCVLFLNTSGQQWYEVERTERIKNCLNPQFSKTFIIDYYFEVVQKLKFGVYDIDNKTIELSDDDFLGECECTLGQIVSSKKLTRPLVMKTGRPAGKGSITISAEEIKDNRVVLFEMEARKLDNKDLFGKSDPYLEFHKQTSDGNWLMVHRTEVVKNNLNPVWRPFKISLNSLCYGDMDKTIKVECYDYDNDGSHDLIGTFQTTMTKLKEASRSSPVEFECINEKKRQKKKSYKNSGVISVKQCEITVECTFLDYIMGGCQLNFTVGVDFTGSNGDPRSPDSLHYISPNGVNEYLTALWSVGLVIQDYDADKMFPAFGFGAQIPPQWQVSHEFPMNFNPSNPYCNGIQGIVEAYRSCLPQIKLYGPTNFSPIINHVARFAAAATQQQTASQYFVLLIITDGVITDLDETRQAIVNASRLPMSIIIVGVGGADFSAMEFLDGDGGSLRSPLGEVAIRDIVQFVPFRQFQNAPKEALAQCVLAEIPQQVVGYFNTYKLLPPKNPATKQQKQ

SEQ ID NO: 73 - DNA, 1210 bp, Homo sapiens
  CDS (543)..(1091)
gctggactgctcgctggccggcagcgcaccgttttgaaggtcctagcccacctgggctggctcacgcgcacgactagccgctcccatacagcacgcccggactctgtcgtcgcttaaggccactcctattctacggctgacccctggtggtcacgtggatctgttcgccacgcaagtctgggtccttcggcgattgaccggggtccttgctgttcgggagcctctcctaagctgcctgttcgcgcgagagtttggaggggcgggtttggggtcggtgtctgattggggctcgcaccgcagcacgctggagtcccgcttaggtaccagttagcgtcaggggagctgggtcaggcggtcgccgggacaccccgtgtgtggcaggcggcgaagcgctctggagaatcccggacagccctgctccctgcagccaggtgtagtttcgggagccactggggccaaagtgagagtccagcggtcttccagcgcttgggccacggcggcggccctgggagcagaggtggagcgaccccattacgctaaagatgaaaggctggggttggctggccctgcttctgggggccctgctgggaaccgcctgggctcggaggagccaggatctccactgtggagcatgcagggctctggtggatgaactagaatgggaaattgcccaggtggaccccaagaagaccattcagatgggatctttccggatcaatccagatggcagccagtcagtggtggaggtgccttatgcccgctcagaggcccacctcacagagctgctggaggagatatgtgaccggatgaaggagtatggggaacagattgatccttccacccatcgcaagaactacgtacgtgtagtgggccggaatggagaatccagtgaactggacctacaaggcatccgaatcgactcagatattagcggcaccctcaagtttgcgtgtgagagcattgtggaggaatacgaggatgaactcattgaattcttttcccgagaggctgacaatgttaaagacaaactttgcagtaagcgaacagatctttgtgaccatgccctgcacatatcgcatgatgagctatgaaccactggagcagcccacactggcttgatggatcacccccaggaggggaaaatggtggcaatgccttttatatattatgtttttactgaaattaactgaaaaaatatgaaaccaaaagt

SEQ ID NO: 74 - protein, 182 aa, Homo sapiens
MKGWGWLALLLGALLGTAWARRSQDLHCGACRALVDELEWEIAQVDPKKTIQMGSFRINPDGSQSVVEVPYARSEAHLTELLEEICDRMKEYGEQIDPSTHRKNYVRVVGRNGESSELDLQGIRIDSDISGTLKFACESIVEEYEDELIEFFSREADNVKDKLCSKRTDLCDHALHISHDEL

SEQ ID NO: 75 - DNA, 4398 bp, Homo sapiens
  CDS (215)..(3121)
gcgggcacgccgccgcctgcgcggttccgggtgctcccggcgcggcgctgcccagggcacacttgaccgaggtcgggctgcggggaaaggcgggtctggggagaaccccgagagacgcggcgtcctccaactcctcctccttctcctcgcggccgccgaagccacagcccgagcccgagcccgagcccgagccggcgccaccgcgcccccggccatggcttttgccaatttccgccgcatcctgcgcctgtctaccttcgagaagagaaagtcccgcgaatatgagcacgtccgccgcgacctggaccccaacgaggtgtgggagatcgtgggcgagctgggcgacggcgccttcggcaaggtttacaaggccaagaataaggagacgggtgctttggttgcggccaaagtcattgaaaccaagagtgaggaggagctggaggactacatcgtggagattgagatcctggccacctgcgaccacccctacattgtgaagctcctgggagcctactatcacgacgggaagctgtggatcatgattgagttctgtccagggggagccgtggacgccatcatgctggagctggacagaggcctcacggagccccagatacaggaggtttgccgccagatgctagaagccctcaacttcctgcacagcaagaggatcatccaccgagatctgaaagctggcaacgtgctgatgaccctcgagggagacatcaggctggctgactttggtgtgtctgccaagaatctgaagactctacagaaacgagattccttcatcggcacgccttactggatggcccccgaggtggtcatgtgtgagaccatgaaagacacgccctacgactacaaagccgacatctggtccctgggcatcacgctgattgagatggcccagatcgagccgccacaccacgagctcaaccccatgcgggtcctgctaaagatcgccaagtcagaccctcccacgctgctcacgccctccaagtggtctgtagagttccgtgacttcctgaagatagccctggataagaacccagaaacccgacccagtgccgcgcagctgctggagcatcccttcgtcagcagcatcaccagtaacaaggctctgcgggagctggtggctgaggccaaggccgaggtgatggaagggatcgaagacggccgggatgagggggaagaggaggacgccgtggatgccgcctccaccctggagaaccatactcagaactcctctgaggtgagtccgccaagcctcaatgctgacaagcctctcgaggagtcaccttccaccccgctggcacccagccagtctcaggacagtgtgaatgagccctgcagccagccctctggggacagatccctccaaaccaccagtcccccagtcgtggcccctggaaatgagaacggcctggcagtgcctgtgcccctgcggaagtcccgacccgtgtcaatggatgccagaattcaggtagcccaggagaagcaagttgctgagcagggtggggacctcagcccagcagccaacagatctcaaaaggccagccagagccggcccaacagcagcgccctggagaccttgggtggggagaagctggccaatggcagcctggagccacctgcccaggcagctccagggccttccaagagggactcggactgcagcagcctctgcacctctgagagcatggactatggtaccaatctctccactgacctgtcgctgaacaaagagatgggctctctgtccatcaaggacccgaaactgtacaaaaaaaccctcaagcggacacgcaaatttgtggtggatggtgtggaggtgagcatcaccacctccaagatcatcagcgaagatgagaagaaggatgaggagatgagatttctcaggcgccaggaactccgagagcttcggctgctccagaaagaagagcatcggaaccagacccagctgagtaacaagcatgagctgcagctggagcaaatgcataaacgttttgaacaggaaatcaacgccaagaagaagttctttgacacggaattagagaacctggagcgtcagcaaaagcagcaagtggagaagatggagcaagaccatgccgtgcgccgccgggaggaggccaggcggatccgcctggagcaggatcgggactacaccaggttccaagagcagctcaaactgatgaagaaagaggtgaagaacgaggtggagaagctcccccgacagcagcggaaggaaagcatgaagcagaagatggaggagcacacgcagaaaaagcagcttcttgaccgggactttgtagccaagcagaaggaggacctggagctggccatgaagaggctcaccaccgacaacaggcgagagatctgtgacaaggagcgcgagtgcctcatgaagaagcaggagctccttcgagaccgggaagcagccctgtgggagatggaagagcaccagctgcaggagaggcaccagctggtgaagcagcagctcaaagaccagtacttcctccagcggcacgagctgctgcgcaagcatgagaaggagcgggagcagatgcagcgctacaaccagcgcatgatagagcagctgaaggtgcggcagcaacaggaaaaggcgcggctgcccaagatccagaggagtgagggcaagacgcgcatggccatgtacaagaagagcctccacatcaacggcgggggcagcgcagctgagcagcgtgagaagatcaagcagttctcccagcaggaggagaagaggcagaagtcggagcggctgcagcaacagcagaaacacgagaaccagatgcgggacatgctggcgcagtgtgagagcaacatgagcgagctgcagcagctgcagaatgaaaagtgccacctcctggtagagcacgaaacccagaaactgaaggccctggatgagagccataaccagaacctgaaggaatggcgggacaagcttcggccgcgcaagaaggctctggaagaggatctgaaccagaagaagcgggagcaggagatgttcttcaagctgagcgaggaggcggagtgcccaaacccctccaccccaagcaaggctgccaagttcttcccctacagttctgcggatgcttcttaacaaccgcccggggctgtggctggcagcttggtgggccccagggccttctccttcattctctgtgaacatgtaactcaggaccccttttccctcttgcgtctgtgccagctcaaatccagcccctcgccctgtgccaccccaactgtgcctgatagacctgccccagcgttcctgacttcttgctggcctgtggagggtgaggtgtaattatttgtcacctgaacctaatgtatattctccttgagccccagatcccttcaagctggaagggatggggctgttggtggggtcagggtccaagaggaatgggtgttctgtggcctcgagtcctctcctgtttgcgaaaataccagttttgctctctgtgggacaaagcactgctgatgaagtccccgtgggctcatccgggctggaattcttggtttttcagccattccctgcagagtcactcaatcatcaagtccctcacagccatttctgttcccagaggagccaggcctgcagctggctgctcaggagatggcctcattcctcctgttctcccagtttgctttccacttaagacaaagccttgtctatgtggggggcggggaccggggaaagagggaggctgaaatgtttattctgcttctcccgtgtttcatgccatctcgcgtcccccttcctgcacatgggtgtgaatgcacacacatacgcgtacacacaggacttggtctgctggcctggcctcttctgcccaggtgggttggaacacgtttgctgcctgagcctgtgccactgagcatgttaggtggagcagttggtgtggcacgtgcggggtgttggcaccggaggcatggagaagcacaggctgtactgccaggctgcgatgcgtgctggcccccgcacaggctcctgtgtgcagggactgattcctcagcacacgaggcttccacaacccagtctgctccatagcactctggcccaccctgtctgcaggtgaaacaggagggctgtttgcctctgccccatccccccgactgtgttcaggagtcccaccttgcatttcagacatgggctggcagtctgttggacttctcttcaggaagaaaaagcatcagggggaaatggaatgcccctgccccaggaacatggcagaagcacaggttctgtacctcagatggactcctgctgggccttcggggtctcagttgacttcccccagattctgattctacagctgcagaatgtatataacgcaataaaagcaaatgtttgaaccaaaaaaaaaaaaaaaa

SEQ ID NO: 76 - protein, 968 aa, Homo sapiens
MAFANFRRILRLSTFEKRKSREYEHVRRDLDPNEVWEIVGELGDGAFGKVYKAKNKETGALVAAKVIETKSEEELEDYIVEIEILATCDHPYIVKLLGAYYHDGKLWIMIEFCPGGAVDAIMLELDRGLTEPQIQEVCRQMLEALNFLHSKRIIHRDLKAGNVLMTLEGDIRLADFGVSAKNLKTLQKRDSFIGTPYWMAPEVVMCETMKDTPYDYKADIWSLGITLIEMAQIEPPHHELNPMRVLLKIAKSDPPTLLTPSKWSVEFRDFLKIALDKNPETRPSAAQLLEHPFVSSITSNKALRELVAEAKAEVMEGIEDGRDEGEEEDAVDAASTLENHTQNSSEVSPPSLNADKPLEESPSTPLAPSQSQDSVNEPCSQPSGDRSLQTTSPPVVAPGNENGLAVPVPLRKSRPVSMDARIQVAQEKQVAEQGGDLSPAANRSQKASQSRPNSSALETLGGEKLANGSLEPPAQAAPGPSKRDSDCSSLCTSESMDYGTNLSTDLSLNKEMGSLSIKDPKLYKKTLKRTRKFVVDGVEVSITTSKIISEDEKKDEEMRFLRRQELRELRLLQKEEHRNQTQLSNKHELQLEQMHKRFEQEINAKKKFFDTELENLERQQKQQVEKMEQDHAVRRREEARRIRLEQDRDYTRFQEQLKLMKKEVKNEVEKLPRQQRKESMKQKMEEHTQKKQLLDRDFVAKQKEDLELAMKRLTTDNRREICDKERECLMKKQELLRDREAALWEMEEHQLQERHQLVKQQLKDQYFLQRHELLRKHEKEREQMQRYNQRMIEQLKVRQQQEKARLPKIQRSEGKTRMAMYKKSLHINGGGSAAEQREKIKQFSQQEEKRQKSERLQQQQKHENQMRDMLAQCESNMSELQQLQNEKCHLLVEHETQKLKALDESHNQNLKEWRDKLRPRKKALEEDLNQKKREQEMFFKLSEEAECPNPSTPSKAAKFFPYSSADAS

SEQ ID NO: 77 - DNA, 1413 bp, Homo sapiens
  CDS (90)..(1067)
agggaataaaggctcagggaccggcagttctactctagagcccaccagcctctcagagcctccggtgactggcctgtgtctccccctggatggacatgtggacggcgctgctcatcctgcaagccttgttgctaccctccctggctgatggtgccacccctgccctgcgctttgtagccgtgggtgactggggaggggtccccaatgccccattccacacggcccgggaaatggccaatgccaaggagatcgctcggactgtgcagatcctgggtgcagacttcatcctgtctctaggggacaatttttacttcactggtgtgcaagacatcaatgacaagaggttccaggagacctttgaggacgtattctctgaccgctcccttcgcaaagtgccctggtacgtgctagccggaaaccatgaccaccttggcaatgtctctgcccagattgcatactctaagatctccaagcgctggaacttccccagccctttctaccgcctgcacttcaagatcccacagaccaatgtgtctgtggccatttttatgctggacacagtgacactatgtggcaactcagatgacttcctcagccagcagcctgagaggccccgagacgtgaagctggcccgcacacagctgtcctggctcaagaaacagctggcggcggccagggaggactacgtgctggtggctggccactaccccgtgtggtccatagccgagcacgggcctacccactgcctggtcaagcagctacggccactgctggccacatacggggtcactgcctacctgtgcggccacgatcacaatctgcagtacctgcaagatgagaatggcgtgggctacgtgctgagtggggctgggaatttcatggacccctcaaagcggcaccagcgcaaggtccccaacggctatctgcgcttccactatgggactgaagactcactgggtggctttgcctatgtggagatcagctccaaagagatgactgtcacttacatcgaggcctcgggcaagtccctctttaagaccaggctgccgaggcgagccaggccctgaactcccatgactgcccagctctgaggcccgatctccactgttgggtgggtggcctgccgggaccctgctcacaggcaggcttttcctccaacctgtggcgctgcagcagggcaggaaggggaaacacagctgatgaactgtggtgccacatgacccttgtggcacagatgcccacgtatgtgaaacacacatggacatgtgtcccagccacagtgttatgctctgtggctggctcacctttgctgagttccggggtgcaatgggggagggagggagggaaagcttcctcctaaatcaagcatctttctgttactgatgttcaataaaagaatagttgccaaggctg

SEQ ID NO: 78 - protein, 325 aa, Homo sapiens
MDMWTALLILQALLLPSLADGATPALRFVAVGDWGGVPNAPFHTAREMANAKEIARTVQILGADFILSLGDNFYFTGVQDINDKRFQETFEDVFSDRSLRKVPWYVLAGNHDHLGNVSAQIAYSKISKRWNFPSPFYRLHFKIPQTNVSVAIFMLDTVTLCGNSDDFLSQQPERPRDVKLARTQLSWLKKQLAAAREDYVLVAGHYPVWSIAEHGPTHCLVKQLRPLLATYGVTAYLCGHDHNLQYLQDENGVGYVLSGAGNFMDPSKRHQRKVPNGYLRFHYGTEDSLGGFAYVEISSKEMTVTYIEASGKSLFKTRLPRRARP

SEQ ID NO: 79 - DNA, 5442 bp, Homo sapiens
  CDS (31)..(1992)
taatagtcacatgacacgtttcccgtcaagatggcggatactctcccttcggagtttgatgtgatcgtaatagggacgggtttgcctgaatccatcattgcagctgcatgttcaagaagtggccggagagttctgcatgttgattcaagaagctactatggaggaaactgggccagttttagcttttcaggactattgtcctggctaaaggaataccaggaaaacagtgacattgtaagtgacagtccagtgtggcaagaccagatccttgaaaatgaagaagccattgctcttagcaggaaggacaaaactattcaacatgtggaagtattttgttatgccagtcaggatttgcatgaagatgtcgaagaagctggtgcactgcagaaaaatcatgctcttgtgacatctgcaaactccacagaagctgcagattctgccttcctgcctacggaggatgagtcattaagcactatgagctgtgaaatgctcacagaacaaactccaagcagcgatccagagaatgcgctagaagtaaatggtgctgaagtgacaggggaaaaagaaaaccattgtgatgataaaacttgtgtgccatcaacttcagcagaagacatgagtgaaaatgtgcctatagcagaagataccacagagcaaccaaagaaaaacagaattacttactcacaaattattaaagaaggcaggagatttaatattgatttagtatcaaagctgctgtattctcgaggattactaattgatcttctaatcaaatctaatgttagtcgatatgcagagtttaaaaatattaccaggattcttgcatttcgagaaggacgagtggaacaggttccgtgttccagagcagatgtctttaatagcaaacaacttactatggtagaaaagcgaatgctaatgaaatttcttacattttgtatggaatatgagaaatatcctgatgaatataaaggatatgaagagatcacattttatgaatatttaaagactcaaaaattaacccccaacctccaatatattgtcatgcattcaattgcaatgacatcagagacagccagcagcaccatagatggtctcaaagctaccaaaaactttcttcactgtcttgggcggtatggcaacactccatttttgtttcctttatatggccaaggagaactcccccagtgtttctgcaggatgtgtgctgtgtttggtggaatttattgtcttcgccattcagtacagtgccttgtagtggacaaagaatccagaaaatgtaaagcaattatagatcagtttggtcagagaataatctctgagcatttcctcgtggaggacagttactttcctgagaacatgtgctcacgtgtgcaatacaggcagatctccagggcagtgctgattacagatagatctgtcctaaaaacagattcagatcaacagatttccattttgacagtgccagcagaggaaccaggaacttttgctgttcgggtcattgagttatgttcttcaacgatgacatgcatgaaaggcacctatttggttcatttgacttgcacatcttctaaaacagcaagagaagatttagaatcagttgtgcagaaattgtttgttccatatactgaaatggagatagaaaatgaacaagtagaaaagccaagaattctgtgggctctttacttcaatatgagagattcgtcagacatcagcaggagctgttataatgatttaccatccaacgtttatgtctgctctggcccagattgtggtttaggaaatgataatgcagtcaaacaggctgaaacacttttccaggaaatctgccccaatgaagatttctgtccccctccaccaaatcctgaagacattatccttgatggagacagtttacagccagaggcttcagaatccagtgccataccagaggctaactcggagactttcaaggaaagcacaaaccttggaaacctagaggagtcctctgaataatggatatacaccaaactggatacccaactttggaaattctgactggtctcagagtctacttgatagaaggactgtttgagaaatgttagaaagcagcagcaattataaggcaaaataggtaatagaaatccaaaaggggattttccttatagaggacattccaagaacacacaacacttataaagcacattgacttgctcattttaaataccaaacttgtgtgactagcagatgaaaattataaatcaattgattctcaggaatgtaactgtggatatgaaagtgatcctatgcattgttaataattccatggtcttaggacaattttgcttaccactttggatctttgtttgaaagccacattttcagaaccagctcatgtattttctttggttatttgaattttattttctttatggacaagagcatcataacataatgataaaaacatatagaaaaactaaagtatcatgatctagatagaagcctgtatttggaatacaggtttgttttgctttctatgttgagaagcattgaaaatgctaatataaaggtgtttagacatttttacgaataagtcagtagtgttttttagtatcagtagtgatatttgtttgtaaattatttacatattgggaaaggtcaataatgaagaaatgaaagaatggaaggaaaggtgtggataggctcattggtatttgaatattctgtctgtcaagtaactagagtattaggctaattgtctacagacctaatttaatcctggctgtcctactgatgatatttggtaaattgtttaacttctgagcctacgtttctccatatataaagtggaaatagtattactatgcctactatatgagaatggttatgaagacaatgcaatgccatgtttagaatcggtttgccagaagaaaattgttttagaattttcccattgacttgatgaatctcaaaagtctcacgcaggaaataattgcttgctgtcagtcaacttccaaacaaaatagatcacagtgtttttattgcattaagctttttaaatgaaaatttcttttttaaagtagtattttatagtcttacagaccagtaaaaatagtaacaagtagaattgtggttttgaaatattactaaggaaaacactctataaattgttttattccttttctggtaggtaaacctgcaaccaccaaggactccaaattgtgtatgacagttggtaagccctaatatacactacataaaaacgttagggctgcctgtaatcccagcactttgggaagctgaggtgggtagatcacttgaggtcaggagttcgagaccagcctggccaacatggcgaaaccctgtctctactaaaaatacaaaattttagctgggtgttatggtgggcacctgtaatcccagctactttgaagatgaggtagcagactcacttgaaccagggaggcggaggttgcagtgagccaagattttgccactgcactccagcctgggtgacagagcaagactctgtctcacaaaaaaaaaaaaagggggctgtacataggcagcaaactaagctgcagtgatgttgcctatatttaaattttctcaaatggccaagctctgatggtctactttatttgagcaatagttgagacttataattgcctataaataaacaaacaaatgaactatttgtttttttttctcacaacatctggcctatattgtctgtcaggaagccatggctccaatgtaaagtacatagttcttacatacttcaactgcagctggtccctgacctcaccaggtttcagagatgttcttaaaggaagccagctgtggcaggtcacagattcatgggaaatggaaagaaccaaggaatatagctcttgcctcacctttctacccactgcagatatagttcaagccagagtaatggaagaacttaacttactagcctctcaggctgctcctatccctacctcccagtgtacagcccctccccatctctttagtcccctttccctcacttccccttttataatgtcacacaaatcagggacagtaggatcacattataacctactttgtcatagggattcgatttttcttatatcaaatcatgtttcctgaaacccagctggggcatatgcactcaatgtctaatacatacttattaatgtaccggatattggccttgcccctggatatcagcaatatattataaaaggttccagtagatgagacgattgagtctgaatacaattgcagtaaattgtgccaataaagatattgtactgttacggtcttagagttaaagccgcttgaatgcagcatgcacattcatgtaaacagacaatcagggtaggcctagaataaccacaaaaattctattggccttactgcagccacctatatgtagaacaatggaggagatagtttgtggtccattattgtaccctgtttcatccattagcatcagaatctctctttcaggtcatttattaaatatgattgaaatgtttaaaagttcctgaacatgattcatgatgattaaaatatcatacaactgataaaagactttaagaactttatatatttcctgttgcctcaaaatgtaacagaaattattcttagagctttgattttagctatcctaattactgcaaataaatatttgttcttatagttttaaatcaaaaagaaaagtcttgttataaaaccttaagcttgaaatcatattaataaaatatattgtacatagtggaaaattttcagtagctaatttaaaatttcagaaaatgctattaaagaattttgattcaagtatttaaactgtttagttatgcatgcttcttattaaccgaaaatgataataccatttagtttagtgatcagtatgagaagcaatacctaatcctatgttgctattgtattttttcctagttggtgtgcctgctcagaaaaacatatactgtatgtgtatacatacctgtgtatatataaaaggtcaatttatatatttttctataggaaaatggagtaacaagttccctatctcccatatttatttgtccatagtaaaatggccacattgatgataatttctagaactagtttctgagattgtcagccctttgtctaaaataatggcagtattaatgattgacttctgtcactgccatagttacctggattgtcagccttggtagcctttgtctaaagtcctaaagagttccaaaaaaaatgtgttgaaatttaattgctaaatagtggttggtgattctttacagtaggaattgtaataattttcttgcaaataagttatttactgctattgatattgaataatttgtcttttattcagatatatttcaaaaagcatgaatatatgattattcataaattgtatactttaccagtaagttttcagaggaaataaagacttttaaatccttttca

SEQ ID NO: 80 - protein, 653 aa, Homo sapiens
MADTLPSEFDVIVIGTGLPESIIAAACSRSGRRVLHVDSRSYYGGNWASFSFSGLLSWLKEYQENSDIVSDSPVWQDQILENEEAIALSRKDKTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIKEGRRFNIDLVSKLLYSRGLLIDLLIKSNVSRYAEFKNITRILAFREGRVEQVPCSRADVFNSKQLTMVEKRMLMKFLTFCMEYEKYPDEYKGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLKTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 81 - DNA, 2376 bp, Homo sapiens
  CDS (28)..(1503)
cgcgcggcccctgtcctccggcccgagatgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcattaggctttatgactgggtgtgtgttgtgtgtatgtaatacataatgtttattgtacagatgtgtggggtttgtgttttatgatacattacagccaaattatttgttggtttatggacatactgccctttcattttttttcttttccagtgtttaggtgatctcaaattaggaaatgcatttaaccatgtaaaagatgagtgctaaagtaagctttttagggccctttgccaataggtagtcattcaatctggtattgatcttttcacaaataacagaactgagaaacttttatatataactgatgatcacataaaacagatttgcataaaattaccatgattgctttatgtttatatttaacttgtatttttgtacaaacaagattgtgtaagatatatttgaagtttcagtgatttaacagtctttccaacttttcatgatttttatgagcacagactttcaagaaaatacttgaaaataaattacattgccttttgtccattaatcagcaaataaaacatggccttaacaaagttgtttgtgttattgtacaatttgaaaattatgtcgggacataccctatagaattactaaccttactgccccttgtagaatatgtattaatcattctacattaaagaaaataatggttcttactggaatgtctaggcactgtacagttattatatatcttggttgttgtattgtaccagtgaaatgccaaatttgaaaggcctgtactgcaattttatatgtcagagattgcctgtggctctaatatgcacctcaagattttaaggagataatgtttttagagagaatttctgcttccactatagaatatatacataaatgtaaaatacttacaaaagtgg

SEQ ID NO: 82 - protein, 491 aa, Homo sapiens [sequence omitted]

SEQ ID NO: 83 - DNA, 971 bp, Homo sapiens
  CDS (139)..(828)
cactcgttgcataaatttgcgctccgccagcccggagcatttaggggcggttggctttgttgggtgagcttgtttgtgtccctgtgggtggacgtggttggtgattggcaggatcctggtatccgctaacaggtcaaaatgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggtcctgcgtctgagaggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtggctgttaattcttcagtcatggcattcgcagtgcccagtgatggcattactctgcactatagccatttgccccaacttaagtttagaaattacaagtttcagtaatagctgaacctgttcaaaatgttaataaaggtttcgttgcatggta

SEQ ID NO: 84 - protein, 229 aa, Homo sapiens
MQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGC

SEQ ID NO: 85 - DNA, 10487 bp, Homo sapiens
  CDS (336)..(5987)
acgcgggggagtgcagctggcaccatgggtgtgctccagagcaacttctgcttgctctgagccccctgccctgcctcccctttcaccatgtttcctctgacaagattttaagtacagcaattcaagaagatttctcctcctaaacgacatttatctgaagtctattgcctcttgattgctggaaaagaatcttaaaatcatttcaaaagtaacttataaacaaacttattaaaagtgatgaaaggagcattgaaattaattagcactaatttttcactgtgccaaagtgtgcagtgtccttcagaggaaacaataacagatctggtgagtgtgccatgccagtggggagaattgaatgtccctcatctccctctttccctagggacattagtcatgagtgccgagtgtgcggggtcacagaagtgggtctttctgcatatgcaaagcacatttctggccagttgcacaaagataacgttgatgcccaggaaagagaagatgatggaaaaggagaggaagaggaagaagattattttgacaaggaactcattcagttaataaaacaaaggaaagaacaaagtcgacaagatgaaccttccaatagcaaccaagaaataaactctgatgacagacgaccccaatggagacgagaagaccgaattccttaccaagacagagagagttacagtcagcctgcatggcatcatcgtggacctccacagcgggattggaaatgggaaaaagatggctttaataatactaggaaaaacagctttccacattctttgaggaatggtggtggaccaagaggacgttccgggtggcataagggtgttgcaggaggctcctcgacttggtttcacaaccatagtaattctggaggtggttggctttcaaatagtggagcagtagattggaatcataatggtacaggaaggaattccagttggctttctgaaggaacaggtggcttttccagttggcatatgaacaacagtaacggaaactggaaatccagtgtacgtagtacaaataattggaattacagtggccctggagacaaatttcaaccaggcagaaacagaaattctaactgtcaaatggaagacatgactatgctatggaacaagaaatctaataagtcaaacaaatacagtcacgacagatataattggcagcggcaagaaaatgacaaacttggtacagttgccacatatagaggtccttctgaaggatttacaagtgataaatttccttcagaaggcttactcgacttcaattttgagcagctggaaagccaaaccactaaacaagcagacactgctacttccaaagttagtggaaagaatggcagtgcggcaagggaaaagcctcgtcgctggacgccttacccttctcagaagactctggatttacagtcgggattgaaagacatcactggtaacaagtcagaaatgatagagaaacctctctttgattttagcttgataactacaggaatacaggagccccaaactgatgaaacacgtaattccccaacacagaaaacacaaaaagaaatacatactggatctcttaatcacaaggcctcttctgattccgctgcttccttcgaggtggtgagacagtgccccactgcagaaaaacctgaacaagagcatacaccaaataaaatgccatcattgaaatccccactccttccatgtccagccactaaatcattgtctcaaaagcaagatccaaagaatatctcaaaaaacaccaaaacaaattttttttcccctggagaacactcaaatccctcgaacaagcccactgtggaagataaccatggtccttacatatccaaactgcgtagttcatgtcctcatgttttaaaagggaataaaagtacatttggctctcaaaagcaatctggtgataatttaaatgatactttacgaaaggccaaagaggtgctacagtgtcatgagtcattgcaaaatccacttcttagcacttctaaaagtaccaggaactatgcaaaagcaagtagaaatgtagaagaatctgaaaaagggtctttgaaaattgagtttcaagtgcacgcactagaagatgaaagtgatggagagacatctgacacggaaaagcatggaacaaaaattggaaccctaggttctgcaactacagaattgttatctggcagcactcgaactgctgatgagaaagaggaggatgaccgcatcctgaagacttctagagagctatccacttccccatgtaatcccatagttcgccagaaagaatctgaattacaaatgacatctgcagccagtccacaccctggcttattgctagacttgaaaacctctctagaagatgcacaggttgatgactctattaaatctcatgtatcttatgaaacagaaggctttgagagtgctagcttggatgcagagcttcaaaaaagtgacatcagtcagccctcgggccctctcctgcctgaactaagtaagcttggctttcctgcctcacttcagagggatctaacccggcacattagtttgaagagcaaaactggagtacaccttcctgagccaaacctcaatagtgcccgccgcattcgcaatattagcggtcaccgaaagagtgagacagagaaggagtctgggctcaagccaaccctacggcagattctaaatgcatctcggagaaatgtcaactgggaacaggtcattcagcaagtaaccaagaaaaagcaagagctgggcaaaggcttacccaggtttggcatagaaatggtaccccttgttcaaaatgaacaagaagccttagatttggatggggaacctgatctgtccagtctagaaggattccagtgggaaggtgtttccatttcctcgtcccctggcttggcaagaaagcgaagcctttctgagagcagcgtgatcatggacagagctccttctgtgtatagcttcttcagtgaggaaggtacaggcaaagaaaatgagccccagcagatggtttcacctagtaactcattgagggctggacagagccagaaagcaaccatgcacctcaaacaagaagtgacacctcgggctgcctccctccgaacaggtgaaagggctgaaaatgttgctacccaaaggcgacatagtgcacaattatcctctgaccatataatacctttgatgcatttggcaaaagacttgaacagccaggagaggtctataccaccgtcagagaatcagaattcccaggagagtaatggagagggaaactgtctgtcatcaagcgcatcctcagcccttgcgatctccagtttagcggatgcagccacagatagtagctgtacctctggtgctgaacaaaatgatggccaaagtattagaaagaaacgaagagccactggagatggatcttctcctgaactcccaagtcttgagagaaaaaataaaagaaggaaaattaaaggaaaaaaagaacgttctcaggttgaccagctgctgaatatttctttaagggaggaagaacttagtaagtcattgcagtgcatggataacaatcttctgcaagcccgtgcagcccttcagacagcttatgtggaagttcagaggctacttatgctcaagcagcagataactatggagatgagtgcactgaggacccatagaatacagattctacagggattacaagaaacatatgaaccttctgagcacccagaccaggttccctgtagcctcacacgagaacgaaggaacagtagatctcaaacatccattgatgccgcactgctgcccactccctttttcccactttttctggagcctccatcttcccatgtgtctccatcacccaccggagcctctcttcaaataaccacgtctcctactttccaaacccatggcagtgtccctgctccagactcatcagttcagattaaacaagagcccatgtctcctgaacaagatgagaatgtgaatgctgtgccaccaagctctgcctgcaatgtgtccaaggaattactggaagctaatagagagatcagtgacagttgtccagtttatccagtcatcactgctagattgtccttaccagagtcaacagaaagtttccatgagcctagccaagaactgaagttttctgtggagcaaagaaataccagaaacagagaaaactctccctcttcccaatcagctggtctttctagcataaataaagaaggggaagagccaaccaaaggcaatagtgggtctgaagcctgtaccagttcttttctaagattgtcttttgcttcagaaacccctttggagaaggaaccccactctccagctgaccagcctgaacaacaggcagaatccactttgacatcagctgagactaggggaagcaagaaaaagaagaaactccggaagaagaaaagtctacgggctgcccatgttcctgagaatagtgacactgaacaggatgttttgactgttaaacctgtaaggaaagtaaaagctggaaagttaattaaaggggggaaagtaacaacctccacttgggaagacagcaggactggtcgggagcaggagagtgtcagagatgagccagatagtgactcgtctctggaagtcctagaaattcctaatcctcagttagaagtagtagccattgattcttcagaatcaggagaagagaaaccagacagcccatctaaaaaggatatttggaactctacagagcaaaacccactagaaacgtctcgttctgggtgtgatgaagttagctctaccagtgaaattggcactcgctataaagatggcatccctgtaagtgtggcagaaactcagactgtgatctcctccataaaaggatcaaagaattcttcagaaatatcttcagagccaggagatgatgatgaacccacagaaggaagctttgagggacaccaagctgccgtaaatgcaattcagatatttgggaacttgctatatacctgttcagcagataaaactgttcgggtttataatctggtgagtcggaaatgtattggtgtctttgagggtcatacctccaaagttaactgcctcctggttactcagacctccgggaagaatgctgccctttacaccgggtccagtgaccataccatccgctgctataatgttaagagccgagagtgtgtggagcagttacagctggaagaccgggtcctctgcctccacagtagatggcgaatcctctatgcgggactggcaaatggcactgtggtcaccttcaacataaagaacaacaaacgacttgagatctttgaatgccatggccctcgggcagtcagctgtcttgctacagctcaggaaggtgcccgaaaactgctggtcgtggggtcttatgactgcacaattagtgtacgcgatgcccggaatggactgctcctcagaactctggagggccatagcaaaaccattctttgcatgaaggtggtgaatgatctcgtgttcagtggctccagtgatcagtcagtccatgctcacaacattcacactggtgagctcgtgcggatctataaaggtcacaatcatgcagtgactgtggtgaatatcctaggaaaagtgatggtgactgcttgcctggataaatttgttcgtgtctatgaattacagtctcatgatcgattacaagtttatggaggacacaaagacatgattatgtgtatgaccatccataaaagcatgatttacactggctgttatgatggcagtattcaggccgtgaggcttaatctgatgcagaattaccgctgttggtggcatggttgctctctgatatttggcgttgtagatcatttaaaacaacacttgctgaccgaccacactaatcccaacttccagactctgaaatgtcgctggaagaactgcgatgcttttttcactgctaggaaaggatccaaacaggatgctgcaggacatattgaacgacatgctgaagatgacagcaaaattgattcatgaagttttttgcctcccacgttgggaagtcattagttgaactattttcacattggccccccacacaggccactctcttccctttcttggtgaagtaaggaaggagaaagtggttactagccaggcatacccctagcataagtctgggcagctctatgggatgataaattacacttttaagttcttgctggaggttttaaatagatttagacaaatgttaaggaaccatacttctctgggacagcatggcatatagtgatatatgctatttgcgttctccagatgtttattgaagatacagatcctaattggttacccagtttgaccctaatcatatgtatattttattgatttcagtttgcaatttttaatttatgttcttatgatggtttaaacctatagtcaggcttttaagtacaagtttgtttaagtgccagactttgaggatcagttttaatttctccatttgtaatagctgggtatttaaactggaagcaaatagttttctttcttaacaattatgtgcagtgtgtgtcactgttttcttgctttatagatagagctggctttaagtgctaaaggacacagtagatttttgacaaacagtggctgctctgctgactatcttttaggaattcaggaagcaaatcacatagtgacaagtccttacagacaccacttctagtaaatctctgtaaatgtggatagaaagctcagtgtgaggcagtgcgtagacctaacgtctacctttacatctgctgttgaacctgggcactacctgttacagatcctgtcaggctgttgaactggaggtgtacctttatcttcctttctagtgtctgaccctgcaagcctaatgttggtttgaacccttttggtttgttcagttgccagcctccatctctcccactgtatggccgtgcctagaccgatggcagccatcgaatattcctctgggctcacggggtgttttccacccccctgcagcaactaagatggtgggggagagggggttagaataaagcatctgaatacagttttcaggacctcaagcagacttcctagagacttgtttctgagacagttctttgccttcacttccctgctagctgggaaagaagagtggagcagagactctgcctggccactgagaacagccaaattcacaaaccctcagtggggctttgtttttggattttctccggactcatcagtaaacctgtagaagtgtcgctttccagccttttgtttctggatcctcaaaactcagaacgtggccgggcgtggtggctcacgcctgtaatcccagcactttgggaggctgaggcaggcagatcacctgaggctaggagttgcagaccagcctggccaacatggcaaaaccccgtctctactaaaaatacaaaaagccgggcgtggtgggcgcctgtaatcccagctgctcaggaggctgaggcaggagaatcgcttgaacccgggaggcagagattgcagtaagctgagatcgtgccactgcactccagcctggtgacagagtgagactccgtctcaaaaaaaaaaaaaaaaactcagcttcttcagagagataaaattgggggaggagcccagggccacataccaagctttgggacatggtgcctcatgctctctgggattgcagaccatccaggtctgtcttcgcccctgttagtgcacatatatccactcacatgtcttccctcaggctatcgggcagggggacttcaccagggggtttatggataggcttcaagagggtctgtgagcccccagaaattgtgtgtgagactgagtatgtgttcttttttccaggaaaggctccagtggttcttacttgatccaaaaaatgtcaagaattaatctaggaggccgggcctggtggctcacacctgtaatcccagctctttgggaggccgaggcgggtggatcactcaaggtcaggcatttgagaccagcctggccaacatagtgaaaccccgtctctattaaaaatacaaaaattagctgggtgtggtggtatgtgcctgtagtctcagctactcaggaggctgagacaggagaatcacttgaaccctggaggtagaagttgcagtgagccaagatcatactactgcactccagcctgggcgacaagagtgaaactccacctcaaaaaaaaaaaaaaaagagttaatctaggagataatgaatggcctagtactagataatatatggccccacaagctcttgacttctgtccttggggaaagccattttgttaaccacactagtgagatttacatgatgcttaatggagaacagagaagatcttgttgcaaaaggtgtattaaatatttgtgctgtttctgtatgagattgagaagcttttcccacctctcacccctatttcctataaggatatccagagaagccaaactgttctgtgggtttgggaatggtcatttcccgggaaaatgcatctggatcgatgactaaacctggcccttttctctgggctgtagtgaagccgcattttcacgctggctggcagtgtgctgagagcctcgaatgctctgcggcgtagtgcccttctgccctgcctgacgatgtatcgaaaagatgagagtgaaggagactttgtgcagcaggaaacgggtaggtgaggtgttgggcagttgtgggaacttctgagagtattacagagtggtagaatcggtaagaactctgatttggacttcgctttggtggaactgtgtgcctatacctgcctgtgtgtgtgcaagtgtgcaggttcctttgtatgtatgtgtacgtgtgggaacctgtgtttgtcatatttttcttcatttcacaaaggctttttttgaagcagtggcagtatgcctttgtttcaagaacacatgaaattcttttaacaccagattagtgtgttaccccaaatgaacggttctagccctctattaagaaataaagggaccataagcattttggctgcttatggctgtgtgttactacttacaagagtcttgaaaattatacagaactttgccttctttttttaatgtcttccacaatgttgtgactgattataaccctgtttcccctcagagaagagctatggctcagggatctgtgttgactctggcatttagtggctttgtgaaggaaagaaaccattaaatgacctgacaaaaactgactcatgtctttaaagtagttgaagccacttttagaatgttactctcggttgcttttgtctaattctaatggcttaaagccaagaaaaccatagtataaatctttttgtgtacctatgctagtgtttaaatggcagttcgttgtgataaagtatcagtcacttcaagttttctgtgaaagtttttaattaggttaaaaataattttggcatacgtctctactcagtttataaagcagttccacaccatgcaaacaaaactctaacagcaaaggttatcttgacctgatgttacttgaatctgagagaaagtcattacttactactgcactcctgcttttgtgggaaaaattttcttcttccccagaagagttaacagttgagtgttcagctcctgacttacctttaatttagaggcaatattattactgtttaagtgattatttgcttaacacaggttttattttgtctttttgctggtagattatacaagggacaactttagtgatgaggcatgtggatgatttggtgggagtaggtatatacttagtggaatggtttattttatggaagagaattcattggaggtgagattgaagtatattgtgaagtgggatttgaacagcacatatgctctggtttgctcaaataactgcagtttatgcttacatctaaattttgtttctgttacctaaacgggagcttctagaattacgaaggccatacctgccacatttataaaataatggttttatccctgcccccaaatcatcaggttcctcgggtttaagagaagtggtctaatagacaggaatagaagttgtggtggaggtgatttgggatagactaggtttccttatgcaagtggatatgagctcctcatagaaaccagacctactgtattagacagtaacctctaacctcacctccaagcccaagtatatggccctgctgggttacctggtgactacatttcccagattcactctaaattcaaggtagtgacccaaataaattgggaaaggagggggtcaaggaggaaaagctgttgaaaatctttctcctatttaagagaaatatatgccctagagctgctccagcacccttggtttctgatttttttaatattgaatatggattagaagaatgaaatcagatgacacctgactgcaaaaatgtttataagcaaatagccttcttacattttggtataaagctgtgaacttcgtaactttgtaaagcaagatataaagcaaatacaagaggaaaataaagtacttttactaattgaaaaaaaaaaaaaaaaaaaa

SEQ ID NO: 86 - protein, 1883 aa, Homo sapiens
MPVGRIECPSSPSFPRDISHECRVCGVTEVGLSAYAKHISGQLHKDNVDAQEREDDGKGEEEEEDYFDKELIQLIKQRKEQSRQDEPSNSNQEINSDDRRPQWRREDRIPYQDRESYSQPAWHHRGPPQRDWKWEKDGFNNTRKNSFPHSLRNGGGPRGRSGWHKGVAGGSSTWFHNHSNSGGGWLSNSGAVDWNHNGTGRNSSWLSEGTGGFSSWHMNNSNGNWKSSVRSTNNWNYSGPGDKFQPGRNRNSNCQMEDMTMLWNKKSNKSNKYSHDRYNWQRQENDKLGTVATYRGPSEGFTSDKFPSEGLLDFNFEQLESQTTKQADTATSKVSGKNGSAAREKPRRWTPYPSQKTLDLQSGLKDITGNKSEMIEKPLFDFSLITTGIQEPQTDETRNSPTQKTQKEIHTGSLNHKASSDSAASFEVVRQCPTAEKPEQEHTPNKMPSLKSPLLPCPATKSLSQKQDPKNISKNTKTNFFSPGEHSNPSNKPTVEDNHGPYISKLRSSCPHVLKGNKSTFGSQKQSGDNLNDTLRKAKEVLQCHESLQNPLLSTSKSTRNYAKASRNVEESEKGSLKIEFQVHALEDESDGETSDTEKHGTKIGTLGSATTELLSGSTRTADEKEEDDRILKTSRELSTSPCNPIVRQKESELQMTSAASPHPGLLLDLKTSLEDAQVDDSIKSHVSYETEGFESASLDAELQKSDISQPSGPLLPELSKLGFPASLQRDLTRHISLKSKTGVHLPEPNLNSARRIRNISGHRKSETEKESGLKPTLRQILNASRRNVNWEQVIQQVTKKKQELGKGLPRFGIEMVPLVQNEQEALDLDGEPDLSSLEGFQWEGVSISSSPGLARKRSLSESSVIMDRAPSVYSFFSEEGTGKENEPQQMVSPSNSLRAGQSQKATMHLKQEVTPRAASLRTGERAENVATQRRHSAQLSSDHIIPLMHLAKDLNSQERSIPPSENQNSQESNGEGNCLSSSASSALAISSLADAATDSSCTSGAEQNDGQSIRKKRRATGDGSSPELPSLERKNKRRKIKGKKERSQVDQLLNISLREEELSKSLQCMDNNLLQARAALQTAYVEVQRLLMLKQQITMEMSALRTHRIQILQGLQETYEPSEHPDQVPCSLTRERRNSRSQTSIDAALLPTPFFPLFLEPPSSHVSPSPTGASLQITTSPTFQTHGSVPAPDSSVQIKQEPMSPEQDENVNAVPPSSACNVSKELLEANREISDSCPVYPVITARLSLPESTESFHEPSQELKFSVEQRNTRNRENSPSSQSAGLSSINKEGEEPTKGNSGSEACTSSFLRLSFASETPLEKEPHSPADQPEQQAESTLTSAETRGSKKKKKLRKKKSLRAAHVPENSDTEQDVLTVKPVRKVKAGKLIKGGKVTTSTWEDSRTGREQESVRDEPDSDSSLEVLEIPNPQLEVVAIDSSESGEEKPDSPSKKDIWNSTEQNPLETSRSGCDEVSSTSEIGTRYKDGIPVSVAETQTVISSIKGSKNSSEISSEPGDDDEPTEGSFEGHQAAVNAIQIFGNLLYTCSADKTVRVYNLVSRKCIGVFEGHTSKVNCLLVTQTSGKNAALYTGSSDHTIRCYNVKSRECVEQLQLEDRVLCLHSRWRILYAGLANGTVVTFNIKNNKRLEIFECHGPRAVSCLATAQEGARKLLVVGSYDCTISVRDARNGLLLRTLEGHSKTILCMKVVNDLVFSGSSDQSVHAHNIHTGELVRIYKGHNHAVTVVNILGKVMVTACLDKFVRVYELQSHDRLQVYGGHKDMIMCMTIHKSMIYTGCYDGSIQAVRLNLMQNYRCWWHGCSLIFGVVDHLKQHLLTDHTNPNFQTLKCRWKNCDAFFTARKGSKQDAAGHIERHAEDDSKIDS